(19) United States
(12) Patent Application Publication
(10) Pub. No.: US 2025/0281456 A1
(43) Pub. Date: Sep. 11, 2025

(54) METHODS OF TREATING CANCER BY INHIBITING BACTERIAL DNAK TO RESTORE ACTIVITIES OF ANTICANCER DRUGS

(71) Applicant: University of Maryland, Baltimore, Baltimore, MD (US)

(72) Inventors: Davide Zella, Tampa, FL (US); Francesca Benedetti, Tampa, FL (US); Robert C. Gallo, St. Petersburg, FL (US)

(21) Appl. No.: 18/854,680

(22) PCT Filed: Apr. 6, 2023
(86) PCT No.: PCT/US2023/065421

§ 371 Date: Oct. 7, 2024

Related U.S. Application Data
(60) Provisional application No. 63/328,362, filed on Apr. 7, 2022.
(60) Provisional application No. 63/344,886, filed on May 23, 2022.

(51) Int. Cl. A61K 31/404; A61K 31/282; A61K 31/555; A61K 31/7105; A61K 31/711; A61K 38/08; A61K 38/10; A61P 35/00; C07K 16/12

(52) U.S. Cl. CPC A61K 31/404; A61K 31/282; A61K 31/555; A61K 31/7105; A61K 31/711; A61K 38/08; A61K 38/10; A61P 35/00; C07K 16/1296

(57) ABSTRACT

The present invention provides a method for increasing the efficacy of an anticancer therapy by administering to a subject in need thereof at least one compound that at least partially blocks the activity of a bacterial DnaK, and pharmaceutical compositions comprising the same.

### FIELD OF THE INVENTION

The field of the invention relates to pharmaceuticals and medicine, in particular therapeutics for the treatment of cancer, or therapeutics which augment cancer therapy.

### BACKGROUND OF THE INVENTION

The cancer-associated microbiota is one of the most significant components of the tumor microenvironment with profound effects on anti-cancer drug response and toxicity, and a number of studies clearly show that the microbiota composition affects the effectiveness of chemotherapeutic drugs (Nejman et al., Science, (2020), 368:973; Poore et al., Nature, (2020), 579:567-74; Maman et al., Nature Reviews Cancer, (2018), 18:359-76; Alexander et al., Nature Reviews Gastroenterology &Amp, Hepatology, (2017); 14:356; Lehouritis et al., Scientific reports, (2015), 5:14554; Helmink et al., Nature medicine, (2019), 25:377-88). In particular, cancer-associated bacteria (CAB) such as Mycoplasma hyorhinis and Fusobacterium nucleatum reduce the efficacy of certain anti-cancer drugs including gemcitabine, cisplatin and 5FU both in vivo and in vitro, though the molecular mechanism(s) involved are still largely unknown (Vande et al., J Biol Chem, (2014), 289:13054-65; Liu et al., PLoS One, (2017), 12:e0184578; Geller et al., Science, (2017), 357:1156-60; et al., Journal of Experimental & Clinical Cancer Research, (2019), 38:14; Yamamura et al., Clinical Cancer Research, (2019), 25:6170-9; Yu et al., Cell, (2017), 170:548-63 e16; Gethings-Behncke et al., Cancer Epidemiology, Biomarkers & Prevention, (2020), 29:539-48; Parhi et al., Nat Commun, (2020), 11:3259). Indeed, a complete map of the microbiota-host-drug interactome in cancer is lacking, mainly due to the difficulty in identifying the contribution of specific bacterial factors to both tumor development, progression and response to therapy. It is clear that understanding how the many players involved in this extremely complex biological system interrelate to prevent optimal drug response would pave the way for the development of effective anti-cancer strategies.

Colorectal and gastric cancers are among the leading cause of tumor-related mortality, both in the United States and worldwide (Sung et al., CA: A Cancer Journal for Clinicians, (2021), 71:209-49). For the treatment of both cancers is widely used a combination therapy regimen comprising platinum-based molecules like cisplatin and/or the anti-metabolite 5-Fluorouracil (5FU) (Saber et al., BMC Cancer, (2018), 18:822; Sui et al., Oncol Lett, (2019), 17:944-50; Arai et al., BMC Cancer, (2019), 19:652). Administration of either molecule results in DNA damage and RNA synthesis inhibition, leading to cell death through a series of cellular events not completely fully understood, but which mainly involves p53 activation (Kong et al., The Journal of biological chemistry, (2014), 289:27134-45; Zamble et al., Proceedings of the National Academy of Sciences, (1998), 95:6163; Bragado et al., Apoptosis: an international journal on programmed cell death, (2007), 12:1733-42; Siddik et al., Oncogene, (2003), 22:7265-79; Hagopian et al., Clinical Cancer Research, (1999), 5:655; Perrone et al., Journal of Clinical Oncology, (2010), 28:761-6; Hientz et al., Oncotarget, (2017), 8:8921-46; Dasari et al., European journal of pharmacology, (2014), 740:364-78; Longley et al., Nature Reviews Cancer, (2003), 3:330; Houghton et al., Clinical cancer research: an official journal of the American Association for Cancer Research, (1995), 1:723-30; Tchounwou et al., Journal of experimental pharmacology, (2021), 13:303-28). Indeed, most anti-cancer drugs rely on the induction or blockage of DNA repair, with consequent activation of p53 followed by apoptosis to exert their function.

It was previously shown that Mycoplasma DnaK, a chaperone protein belonging to the Hsp70 family, binds to USP10 (ubiquitin carboxyl-terminal hydrolase 10), a regulator of p53 stability (Zella et al., Proceedings of the National Academy of Sciences, (2018), 115:E12005-E14). This binding in turn reduces the activities of p53, an essential transcription factor that promotes cell cycle blockage and apoptosis in the presence of extensive DNA damage (Zella et al., Proceedings of the National Academy of Sciences, (2018), 115:E12005-E14; Benedetti et al., International journal of molecular sciences, (2020), 21(4); Hafner et al., Nature Reviews Molecular Cell Biology, (2019), 20:199-210). Of note, DnaK/HSP70 may be released by the bacteria and then taken up by uninfected cells or directly translocated into the eukaryotic cells upon attachment or invasion (Zella et al., Proceedings of the National Academy of Sciences, (2018), 115:E12005-E14; Benedetti et al., International journal of molecular sciences, (2020), 21(4); Bendtsen et al., BMC microbiology, (2005), 5:58; Carrio et al., J Bacterial, (2005), 187:3599-601; Mambula et al., Methods, (2007), 43, 3:168-75; Theriault et al., J Immunol, (2006), 177:8604-11; Costa et al., Nature Reviews Microbiology, (2015), 13:343; Holland et al., Biochinica et Biophysica Acta (BBA)—Molecular Cell Research, (2004), 1694:5-16; Curreli et al., International Journal of Molecular Sciences, (2021), 22:3885).

What is needed is a greater understanding regarding various pathogen-cancer relationships and why certain drugs appear to fail in order to devise new compositions and methods that are useful to treat cancer or enhance anti-cancer therapy. The foregoing description of the background is provided to aid in understanding the invention, and is not admitted to be or to describe prior art to the invention.

### SUMMARY OF THE INVENTION

It is to be understood that both the foregoing general description of the invention and the following detailed description are exemplary, and thus do not restrict the scope of the invention.

In one aspect, the invention provides a method for increasing the efficacy of an anticancer therapy in a subject, comprising administering to the subject in need thereof an effective amount of at least one compound that at least partially blocks the activity of a bacterial DnaK.

In some embodiments, the administration of the compound restores or enhances the activity of anticancer therapies, such as chemotherapeutic drugs, that work via the p53 pathway, cyclin pathway, or other pathways.

In some embodiments, the compound blocks the interaction of DnaK and PARP1.

In some embodiments, the compound blocks the interaction of DnaK and USP10.

In some embodiments, the compound blocks the activation by DnaK of one or more protein kinases. The kinase is not particularly limiting, but includes kinases associated with cancer. In some embodiments, the kinases are selected from the group consisting of ERK1, EGFR, PDGFRB, SRC, p38a, p38b, ERK2, HCK, FAK, RSK1, RSK2, RSK3, LYN, LCK, GSK3A, GSK3B, MSK1, MSK2, PYK2, PRKAA1, PRKAA2, mTOR, AKT1, AKT2, AKT3, WNK1, RPS6KB1, YES, FYN, FGR and combinations thereof.

In some embodiments, the compound inhibits the expression of DnaK.

In some embodiments, the compound is telaprevir (also known as VX-950).

In some embodiments, the compound is zafirlukast.

In some embodiments, the compound is a peptide comprising an amino acid sequence selected from GNNRPVYIPQPRPPHPRL (SEQ ID NO:1) and VDKGSYLPRPTPPRPIYNRN (SEQ ID NO:2) and variants thereof.

In some embodiments, the compound is ARV-1502.

In some embodiments, the compound is a DnaK antibody. In some embodiments, the antibody is a monoclonal antibody.

In some embodiments, the compound is a nucleic acid that inhibits the expression of DnaK.

In some embodiments, the nucleic acid is an RNA, a DNA, or a combination thereof. In some embodiments, the nucleic acid is a ribozyme.

In some embodiments, the compound is administered to a subject by topical, intravenous, subcutaneous, intramuscular, intracutaneous, transcutaneous, intrathecal, intranasal, intra-arterial, rectal, intragastric, parenteral, or oral administration.

In some embodiments, the method further comprises administering the anticancer agent to the subject.

In some embodiments, the anticancer agent is selected from the group consisting of oxaliplatin, cisplatin, docetaxel, etoposide, palbociclib, lenalidomide, and bortezamib.

In some embodiments, the bacterial DnaK is one or more of DnaK from Mycoplasma, Mycoplasma fermentans, Heliobacter pylori, Fusobacterium nucleatum, and Chlamydia trachomatis.

In some embodiments, the anticancer therapy is cisplatin and the compound that at least partially blocks the activity of bacterial DnaK is telaprevir. In some embodiments, the DnaK is from M. fermentans, F. nucleatum, or from both.

In some embodiments, the method further comprises detection of a bacteria prior to administering to the subject in need thereof an effective amount of at least one compound that at least partially blocks the activity of a bacterial DnaK.

In some embodiments, the bacteria is selected from Mycoplasma, Mycoplasma fermentans, Heliobacter pylori, Fusobacterium nucleatum, Chlamydia trachomatis, and combinations thereof.

In some embodiments, the method further comprises detection of a bacterial DnaK in the subject prior to administering to the subject in need thereof an effective amount of at least one compound that at least partially blocks the activity of a bacterial DnaK.

In some embodiments, the bacterial DnaK is one or more of DnaK from Mycoplasma, Mycoplasma fermentans, Heliobacter pylori, Fusobacterium nucleatum, and Chlamydia trachomatis.

In another aspect, the invention provides a pharmaceutical composition formulated for administration as an infusion, comprising an effective amount of at least one compound that at least partially blocks the activity of a bacterial DnaK, in combination with a pharmaceutically acceptable carrier. In some embodiments, the at least one compound that at least partially blocks the activity of a bacterial DnaK is selected from the group consisting of telaprevir (also known as VX-950), zafirlukast, and combinations thereof. In some embodiments, the pharmaceutical composition further comprises an effective amount of an anticancer agent. In some embodiments, the anticancer agent is selected from the group consisting of oxaliplatin, cisplatin, docetaxel, etoposide, palbociclib, lenalidomide, bortezamib, and combinations thereof.

In another aspect, the invention provides a screening assay to detect test compounds that are able to disrupt an interaction between DnaK and one or both of PARP1 or USP10.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Mycoplasma infection induces tumorigenesis in SCID mice. A. Mycoplasma infection in SCID mice. Inverted Kaplan-Meyer formula was used to generate a plot of the time to tumor development. CB17.SCID (N=18) and NOD/SCID (N=12) mice were infected with a strain of Mycoplasma fermentans isolated in our laboratory. The experiments were carried out for about 19-20 weeks after infection, until the animals reached an age of about 27 weeks. Out of 30 infected animals, a total of 12 animals (8 CB17.SCID and 4 NOD/SCID) developed tumors within 27 weeks of age, starting at about 8 weeks after infection. The CB17.SCID animals belonged to a colony maintained in our animal facility under pathogen-free conditions, NOD/SCID and NOD/SCID gamma (NSG) mice were obtained from the Jackson Laboratory in Bar Harbor Maine. Young animals (about 6 weeks old) were infected by intra-peritoneal (i.p.) injection with Mycoplasma (10⁷ pfu). Tumor development was observed in animals infected with Mycoplasma grown in either aerobic or anaerobic conditions. As early as 7 weeks post infection (p.i.), the spleen and lymph nodes were enlarged in animals infected with Mycoplasma. In some animals tumor cells colonized the vestigial thymic area, and necropsy showed an enlarged tumor mass. About 30% of the animals died of wasting within 30 weeks of infection. Age-matched uninfected CB17.SCID (N=9) and NOD/SCID (N=9) animals were kept in adjacent cages as controls. Control, uninfected CB17.SCID mice had a lifespan of about 40-50 weeks, while NOD/SCID mice had a lifespan of 38-45 weeks. Only 1 CB17.SCID developed a spontaneous tumor at about 26 weeks of age. Spontaneous T-cell lymphoma was observed in more than 40% of both the CB17.SCID animals and NOD/SCID animals after 33 weeks of age. As a further control, we used NSG mice, which are resistant to lymphoma development even after sub-lethal irradiation treatment. None of the infected NSG animals (N=8) developed tumors during the time of the experiment. In some experiments (N=10) we also used the prototype M. fermentans PG18 grown under standard conditions. Seven animals died of wasting within 30 weeks after infection, and none of the remaining animals developed lymphoma. Eight animals were injected with non-viable Mycoplasma, and none developed lymphoma up to 28 weeks of age (see also Materials and Methods. B and C. Splenomegaly and enlarged lymph-nodes in Mycoplasma-infected tumor mice. Spleens from Mycoplasma infected animals and uninfected animals were collected and compared to determine size increase. Uninfected spleens showed very little size variation compared to each other and were considered as reference in determining the size of spleens from infected animals. In C, spleens from a total of 7 infected animals and 5 uninfected animals were analyzed. SD is shown. Student t test was used to test for statistical difference. *: p<0.01. D tumor infiltration of Mycoplasma-infected mice. D1. Hematoxylin and Eosin (H&E) stain section of a peripheral lymph node showing increased cellularity of tumor infiltration. There is increased vascularity noted by numerous slits (low-power). D2. H&E stain section of a peripheral lymph node of tumor infiltration. Note the prominent follicular hyperplasia with a poorly defined medullar zone (high-power). D3. H&E stain section of the spleen with prominent red pulp showing increased cellularity of tumor infiltration. There is increased vascularity noted by numerous slits (low-power). D4. H&E stain section of the spleen with a tumor infiltration (high-power).

FIG. 2. DnaK negatively affects p53 activities and Mycoplasma infection reduces the effect of anti-cancer drugs. (A) DnaK reduces p53-associated activities in HCT116 cells. Levels of p53, p21, Bax and PUMA proteins were analyzed in control, vector and DnaK transfected cells at different time-points (2, 8 and 16 hours). Expression of DnaK was verified using the anti-V5 antibody. NT: not transfected; VT: vector transfected; DnaK: DnaK transfected; M: media; D: DMSO; N: Nutlin. β-act: β-actin. Band intensity was measured by densitometric analysis. Numbers above bands indicate fold increase above level of nutlin-treated DnaK-transfected cells, normalized for the levels of β-actin. (B) DnaK increases cell cycle progression. HCT116 cells were transfected with a DnaK-expressing vector and subsequently analyzed for cell cycle progression. Data were collected 16-24 hours after transfection. Results represent mean and standard deviations of 5 different experiments. Fisher's exact t-test was used to test for statistical difference. *: p<0.02; **: p<0.05 (C) Mycoplasma infection reduces the effect of chemotherapeutic drugs 5FU and Nutlin. HCT116 cells were infected with Mycoplasma. Results are expressed as percent cell viability over control (uninfected cells in media alone were considered as 100%). Mean difference is shown. *=p<0.001—calculated using Poisson regression.

FIG. 3. DnaK Immunoprecipitates USP10 and reduces stability of p53 upon DNA damage. (A) Immunoprecipitation analysis shows binding of DnaK to USP10. HCT116 cells were transfect with DnaK-V5, and immunoprecipitation was performed using anti-V5 antibody and IgR: antibody isotype control (Rabbit). After washing, the immunoprecipitated products were loaded on an acrylamide gel, as described in materials and methods. αUSP10: antibody anti-USP10. (B) Dnak induces p53 ubiquitination. HCT116 were co-transfected with Dnak-V5 together with HA-Ubiquitin (HA-Ub) and Flag-p53 expression vectors. V5-empty vector was used as negative control. Cells were treated with the proteasome inhibitor MG132 for 5 h before harvest. Flag-p53 and IgG isotype control immunoprecipitates (IP) or whole cell lysates (input) were immunoblotted with anti-Flag and anti-HA. Input lysates were also immunobloted with anti-V5 and anti-beta actin antibodies. Immunoblot is representative of two independent experiments. MG132: proteasome inhibitor. (C) DnaK regulates p53 stability. CT116 transfected with Dnak-V5 or the control vector were treated with cycloheximide (CHX) (0.1 mg/ml), and harvested at time points 0, 1 h, 2 h and 4 h. Cell lysates were then blotted with anti-p53, anti-v5 and anti-β actin antibodies.

FIG. 4. Interaction of DnaK with proteins implicated in the DNA-repair pathway and with DNAJA1. HCT116 cells were transfect with DnaK-V5, and immunoprecipitation was performed using anti-V5 antibody and IgR: antibody isotype control (Rabbit). After washing, the immunoprecipitated products were loaded on an acrylamide gel, as described in materials and methods. (A) Immunoprecipitation analysis shows binding of DnaK to PARP1. αPARP1: antibody anti-PARP1 (B) Measurement of catalytic activity of PARP1 shows reduction of histone-PARylation in the presence of DnaK. Purified PARP1 and DnaK were incubated together, and PARP1 activity was subsequently analyzed, according to the protocol described in materials and methods. O.D.: optical density (C) Immunoprecipitation analysis shows binding of DnaK to DNA-PK_{CS}. αDNA-PK_{CS}: antibody anti DNA-PK_{CS}. (D) Immunoprecipitation analysis shows binding of DnaK to DNAJA1. αDNAJA1: antibody anti-DNAJA1. IgR: antibody control-Rabbit; V5: tag for DnaK; IP:immunoprecipitation.

FIG. 5. Intracellular uptake of exogenous DnaK-V5 by Mycoplasma-free HCT116 cells. Confocal images of exogenous DnaK-V5 protein of M. fermentans in HCT116 cell lines treated with DnaK-V5 protein and untreated. The figures show the collected Z-stacks of corresponding gallery of images, each presenting 0.5 μm thick slide. Insert figure in the lower right corner is a corresponding constructed 3D presentation of the protein uptake. Primary labelling used a mouse monoclonal-antibody anti-V5 and then labelled with FITC fluoresce-labelled secondary antibody. (A): Nuclear localization. (B): perinuclear localization. (C): Negative control. Primary and secondary antibodies alone without DnaK-V5 protein and (D): Negative control. No antibodies and no protein. DAPI staining was used for nuclei detection. Bar is 5 m in A and B, and 20 m in C and D.

FIG. 6. Phylogenetic analysis of bacterial DnaKs. Published bacterial amino acid DnaKs sequences were used to construct this tree by using the MEGA 7.02.20 software (see ref. S3). Beside DnaKs from several strains of E. coli, other DnaKs from intracellular pathogens currently associated with some human cancers are indicated. Bss: base substitutions per site.

FIG. 7. Telaprevir restores anticancer activity of cisplatin in the presence of DnaK from M. fermentans and F. nucleatum.

FIG. 8. Telaprevir restores anticancer activity of cisplatin in the presence of DnaK from M. fermentans and F. nucleatum.

FIG. 9. Zafirlukast restores anticancer activity of cisplatin in the presence of DnaK from M. fermentans and F. nucleatum in HCT116 cells (adenocarcinoma cell line).

FIG. 10. A) Distribution of the number of samples across cancer types retrieved from TCGA (n=10,293). B) Post-filtering distribution of the primary solid tumor and solid tissue normal samples across cancer types included in the analyses.

FIG. 11. Effect of eM-DnaK and ARV-1502 on viability of HCT116 and AGS cell line treated with cisplatin and 5FU. Cisplatin 25 μM (A-C) and 5FU 75 μM (B-D) were added to each well with the indicated cell line alone or in combination with eM-DnaK. Parallel wells of untreated cells were used as negative control. Cell viability was assessed by using the trypan blue assay. Percentage of alive cells for each treatment are calculated as percentage using the untreated cell as reference. Results are representative of 3 independent experiments for each treatment. Statistical differences were tested using Student's t test. All statistical tests were two sided. p=***<0.001, **<0.01, *<0.05, ns=not significant. Treatment with ARV-1502 alone showed on average 5-8% reduction in cell viability (data not shown to maintain a clearer visibility of the results).

FIG. 12. ARV-1502 increases anti-cancer activity of cisplatin and 5FU in cells from a murine primary cancer constitutively expressing DnaK. A) Hematoxylin and Eosin (H&E) staining of a spontaneous mass removed from the abdomen of a DnaK positive mouse. The normal architecture is effaced by unencapsulated, poorly demarcated, densely cellular neoplasm composed of round cells arranged in sheets. Neoplastic cells have variably distinct cell borders, a scant amount of eosinophilic cytoplasm, a round, occasionally indented nucleus with finely stippled chromatin and one variably prominent nucleolus. Anisocytosis and anisokaryosis are moderate, and mitotic count is up to 7 in a single high-power field (2.32 mm²). These findings are consistent with a round cell neoplasia. The images of the section were taken at 4× (top) and 40× (bottom). B) Western Blot analysis confirms expression of DnaK-V5 in the murine primary cancer cells isolated from the spontaneous tumor. Both eM-DnaK and DnaK expressed in cancer cells were fused to a V5 peptide sequence for convenient detection. eM-DnaK has been used as positive control for antibody detection. Cells isolated from a spleen of a DnaK^{-/-} mouse were used as negative control. Upper part: membrane probed with anti-V5 antibody recognizing DnaK-V5. Lower part: membrane probed with an anti-GAPDH antibody. Markers specifying the molecular weight (MW) are indicated. C) Viability assay of primary murine cancer cells treated with anti-cancer drugs with or without ARV-1502. Cells from the spontaneous tumor mass (round cell neoplasia) detected in a DnaK positive mouse were isolated and then treated with the anti-cancer drugs, cisplatin (25 μM) or 5FU (75 μM). In parallel, the cells were also treated with ARV-1502. We assessed cell viability by using the trypan blue assay. Percentage of alive cells for each treatment are calculated as percentage using untreated cell as reference. The results are representative of two independent experiments using primary cells from two different spontaneous tumors. Statistical differences were tested using Student's t test. All statistical tests were two sided. p=***<0.001, **<0.01.

FIG. 13. A) Distribution of the 30 most abundant bacterial taxa (species level) for each disease type for both Primary Solid Tumor and Normal Solid Tissue. B) Heatmap displaying relative abundance values of bacteria identified in Primary Solid Tumor and Normal Solid Tissue. C) Genus distribution of the top 50 Blast hits for the 3 DnaK domains of Mycoplasma. Domain 1 (NDB) aa1-392, domain 2 (SBD) aa392-507 and domain 3 (α-helical domain) aa508-638, as described (49). Fusobacterium is indicated by an arrow.

FIG. 14. Effect of eF-DnaK and ARV-1502 on viability of HCT116 and AGS cell line treated with cisplatin and 5FU. Cisplatin 2 μM (A-C) and 5FU 75 μM (B-D) were added to each well with the indicated cell line alone or in combination with eF-DnaK. Parallel wells of untreated cells were used as negative control. Cell viability was assessed by using the trypan blue assay. Percentage of alive cells for each treatment are calculated as percentage using the untreated cell as reference. Results are representative of 3 independent experiments for each treatment. Statistical differences were tested using Student's t test. All statistical tests were two sided. p=*<0.05, **<0.01, ***<0.001. Treatment with ARV-1502 alone showed on average 5-8% reduction in cell viability (data not shown to maintain a clearer visibility of the results).

FIG. 15. Distribution of Fusobacterium and Mycoplasma bacteria across cancer types. The distribution of both Fusobacterium and Mycoplasma bacteria was determined in the different cancer samples belonging to the TCGA data set, as described in Materials and Methods. The average relative abundance and distribution of each bacterium in primary solid tumors and solid tissue normal are indicated.

FIG. 16. Graphical representation depicting the inhibitory effect of exogenous DnaK (in blue) on the activity of anti-cancer drugs Cisplatin and 5FU in cancer cells. Adding an inhibitor of DnaK ATP-ase activity restores the activity of the anti-cancer drugs. The figure has been created with BioRender.com.

FIG. 17. A) Direct binding of eM-DnaK to ARV-1502 as determined by surface plasmon resonance (SPR). Association of ARV-1502 at different concentrations on 2274.9 response units of eM-DnaK immobilized on a CM5 biosensor chip proceeded at a flow rate of 35 l/min for 250 sec, followed by a 600 sec dissociation in HBS-EP. A preliminary kinetic analysis yielded a Kd value of 1.899e⁻⁶ M. B) ARV-1502 binds to eM-DnaK and does not prevent eM-DnaK entry into HCT116 cells. eM-DnaK was incubated for 3 hours with ARV-1502 and then added to HCT116 cells. After 24 hours of incubation cells were treated for 48 hours with cisplatin. Western blotting analysis shows that eM-DnaK is able to enter into HCT116 cell line despite the binding of ARV-1502 to DnaK and the treatment with cisplatin. Cells not treated with eM-DnaK, ARV-1502 and cisplatin were used as control.

FIG. 18. Top panel—Total number of reads sequenced per cancer type and tissue type (primary solid tumor and solid tissue normal). Bottom panel—Number of 16S sequences per cancer type and tissue type (primary solid tumor and solid tissue normal). The total number of reads was determined in the different cancer samples belonging to the TCGA data set.

### DETAILED DESCRIPTION OF THE INVENTION

The present invention is based on the surprising discovery that DnaK from bacteria can block the activity of certain anti-cancer drugs. Moreover, the present inventors have discovered that blocking the activity of DnaK can at least partially restore the anti-cancer activity of such drugs.

The present inventors have isolated and characterized a strain of human Mycoplasma able to induce lymphoma in a Severe Combined Immuno-Deficient (SCID) mouse model, consistent with a previously described lymphomagenesis dependent upon reduced p53 activity. It is demonstrated that this Mycoplasma's DnaK, belonging to the HSP70 chaperone family, binds to human PARP1 and reduces its catalytic activity. PARP1 activates and recruits to the site of DNA damage important components of the DNA-repair complex. Moreover, this DnaK also binds human USP10 (ubiquitin carboxyl-terminal hydrolase 10, an important regulator of p53 stability), reducing p53 stability and anticancer functions. This indicates that, in cells where the DnaK is present, PARP1 and p53 activities will be reduced, increasing the likelihood of DNA mutations and consequent malignant transformation.

Mycoplasma was abundantly detected early in infected mice, but only low copy numbers of Mycoplasma DnaK DNA sequences were found in primary and secondary tumors, suggesting a “hit and run/hide” mechanism of transformation, in which the critical events have occurred previous to cancer detection.

The present inventors have discovered that DnaK reduces the efficacy of anti-cancer drugs [e.g., 5-fluoracil (5FU) and nutlin] that depend on p53 to exert their effect.

Other bacteria associated with human cancers (including certain Mycoplasmas, H. pylori, F. nucleatun and C. thrachoniatis) have closely related DnaKs, indicating a potential common mechanism of cellular transformation. The data thus indicate that Mycoplasmas, and perhaps certain other bacteria with closely related DnaK sequences and structure, have oncogenic potential mediated through inhibition of DNA repair mechanisms and p53 function, with consequent accumulation of mutations, a greatly increased chance of cellular transformation and resistance to anti-cancer drugs.

While not wishing to be bound by theory, it is believed that (1) DnaK from bacteria associated with human cancers reduces the efficacy of anti-cancer drugs in vitro by binding to USP10 and hampering p53 activities; (2) that tissue-associated bacteria from cancer patients express DnaK proteins that reduce efficacy of anti-cancer drugs that depend on p53 activities; and (3) that RNA levels of DnaKs in tissue-associated bacteria from cancer patients directly correlate with poor response to anti-cancer therapy.

Reference will now be made in detail to embodiments of the invention which, together with the drawings and the following examples, serve to explain the principles of the invention. These embodiments describe the invention in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

### Therapeutic Methods

In one embodiment, the invention provides a method for increasing the efficacy of an anticancer therapy in a subject, comprising administering to the subject in need thereof an effective amount of at least one compound that at least partially blocks the activity of a bacterial DnaK.

In some embodiments, the administration of the compound restores or enhances the activity of anticancer therapies, such as chemotherapeutic drugs, that work via the p53 pathway, cyclin pathway, or other pathways.

As used herein, “treat” and all its forms and tenses (including, for example, treating, treated, and treatment) can refer to therapeutic or prophylactic treatment. In certain aspects of the invention, those in need thereof of treatment include those already with a pathological condition of the invention (including, for example, a cancer), in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition, but may not be a complete cure of the pathological condition. In other certain aspects of the invention, those in need thereof of treatment include, those in which a pathological condition is to be prevented, in which case treating refers to administering a therapeutically effective amount of a composition to a subject (including, for example, a human or other mammal in need of treatment) at risk of developing a disease or condition such as cancer.

In accordance with the invention, a “therapeutically effective amount” or “effective amount” is administered to the subject. As used herein a “therapeutically effective amount” or “effective amount” is an amount sufficient to decrease, suppress, or ameliorate one or more symptoms associated with the disease or condition.

As used herein, the term “subject” is not limiting and is used interchangeably with patient. In some embodiments, the term subject refers to animals, such as mammals and the like. For example, mammals contemplated include humans, primates, dogs, cats, sheep, cattle, goats, pigs, horses, chickens, mice, rats, rabbits, guinea pigs, and the like.

In some embodiments, inhibition of bacterial DnaK can also be used to enhance the efficacy of cancer treatment in cancer patients who have been treated with antibiotics, since antibiotics can sometimes decrease the efficacy of immunotherapy and other cancer treatments.

In some embodiments, the method further comprises detection of a bacteria prior to administering to the subject in need thereof an effective amount of at least one compound that at least partially blocks the activity of a bacterial DnaK. The bacteria is not limiting provided it expresses a DnaK. In some embodiments, the bacteria is selected from Mycoplasma, Mycoplasma fermentans, Heliobacter pylori, Fusobacterium nucleatum, Chlamydia trachomatis, and combinations thereof.

In some embodiments, the method comprises detection of a bacterial DnaK in the subject prior to administering to the subject in need thereof an effective amount of at least one compound that at least partially blocks the activity of a bacterial DnaK. In some embodiments, the bacterial DnaK is one or more of DnaK from Mycoplasma, Mycoplasma fermentans, Heliobacter pylori, Fusobacterium nucleatum, and Chlamydia trachomatis. In some embodiments, the bacteria and/or DnaK is detected by PCT, such as qPCR as described herein.

### Bacterial DnaK Blocking Compounds

A compound that at least partially blocks the activity of bacterial DnaK as used herein can also be referred to as an “antagonist.” The term “antagonist” refers to a biological or chemical agent that acts within the body to reduce the activity of another chemical or biological substance. In the present invention, the antagonist can block, inhibit, reduce and/or decrease the activity of DnaK of a cell. In some embodiments of the invention, without being bound by theory, the antagonist combines, binds, or associates with DnaK such that at least some portion of the DnaK is blocked, meaning reduced activity with respect to the activity in the methods herein. In certain embodiments, the antagonist combines, binds and/or associates with a protein that cooperates with DnaK and is necessary for inhibition of anti-cancer therapy. The terms “antagonist” or “inhibitor” can be used interchangeably. In some embodiments, the DnaK antagonist reduces or inhibits the interaction of DnaK with PARP1, thereby restoring PARP1 catalytic activity. In some embodiments, the DnaK antagonist reduces or inhibits the interaction of DnaK and USP10.

The compound that can be used to at least partially block the activity of bacterial DnaK is not limiting. In some embodiments, the compound that at least partially blocks the activity of bacterial DnaK restores or enhances the activity of an anticancer therapy (e.g., chemotherapeutic drugs) that works via the p53 pathway, cyclin pathway, or other pathways.

In some embodiments, compounds for blocking bacterial DnaK that can be used include, but are not limited to, peptides that inhibit bacterial DnaK, e.g., peptides from AnaSpec, Inc., such as apidaecin IB (amino acids GNNRPVYIPQPRPPHPRL) (SEQ ID NO:1) and pyrrhocoricin (amino acids VDKGSYLPRPTPPRPIYNRN) (SEQ ID NO:2) and derivatives thereof, as well as small molecules that inhibit DnaK.

In some embodiments, the compound is ARV-1502.

In some embodiments, the compound is a peptidomimetic compound. In some embodiments, the class of compounds is described in U.S. Pat. No. 7,820,671, which is incorporated by reference herein. In some embodiments, the compound is a peptidomimetic compound of formula I:

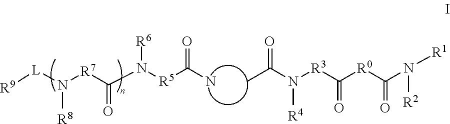

wherein: R⁰ is a bond or difluoromethylene; R¹ is hydrogen, optionally substituted aliphatic group, optionally substituted cyclic group or optionally substituted aromatic group; R² and R⁹ are each independently optionally substituted aliphatic group, optionally substituted cyclic group or optionally substituted aromatic group; R³, R⁵ and R⁷ are each independently (optionally substituted aliphatic group, optionally substituted cyclic group or optionally substituted aromatic group)(optionally substituted methylene or optionally substituted ethylene), optionally substituted (1,1- or 1,2-)cycloalkylene or optionally substituted (1,1- or 1,2-)heterocyclylene; R⁴, R⁶, R⁸ and R¹⁰ are each independently hydrogen or optionally substituted aliphatic group;

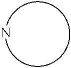

is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-(N(R⁸)—R⁷—C(O)—)ₙN(R⁶)—R⁵—C(O)—N moiety and to which the C(O)—N(R⁴)—R³—C(O)C(O)NR²R¹ moiety is attached; L is —C(O)—, —OC(O)—, —NR¹⁰C(O)—, —S(O)₂—, or —NR¹⁰S(O)₂—; and n is 0 or 1, or a pharmaceutically acceptable salt or prodrug thereof, or a solvate of such a compound, its salt or its prodrug, provided when

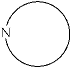

is substituted

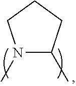

then L is —OC(O)— and R⁹ is optionally substituted aliphatic, or at least one of R³, R⁵ and R⁷ is (optionally substituted aliphatic group, optionally substituted cyclic group or optionally substituted aromatic group)(optionally substituted ethanediyl), or R⁴ is optionally substituted aliphatic.

In some embodiments, the compound is a peptidomimetic compound of formula II

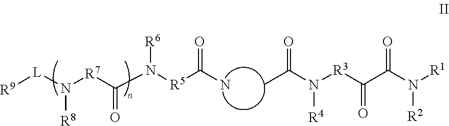

wherein: R¹ is hydrogen, optionally substituted aliphatic group, optionally substituted cyclic group or optionally substituted aromatic group; R² and R⁹ are each independently optionally substituted aliphatic group, optionally substituted cyclic group or optionally substituted aromatic group; R³, R⁵ and R⁷ are each independently (optionally substituted aliphatic group, optionally substituted cyclic group or optionally substituted aromatic group)(optionally substituted methanediyl or optionally substituted ethanediyl); R⁴, R⁶, R⁸ and R¹⁰ are each independently is hydrogen or optionally substituted aliphatic group;

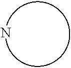

is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-(N(R¹)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰C(O)—, —S(O)₂—, or —NR¹⁰S(O)₂—; and n is 0 or 1, or a pharmaceutically acceptable salt or prodrug thereof, or a solvate of such a compound, its salt or its prodrug, provided when

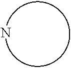

is substituted

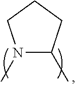

then L is —C(O)— and R⁹ is optionally substituted aliphatic, or at least one of R³, R⁵ and R⁷ is (optionally substituted aliphatic group, optionally substituted cyclic group or optionally substituted aromatic group)(optionally substituted ethanediyl), or R⁴ is optionally substituted aliphatic.

In some embodiments, the compound is telaprevir (also known as VX-950) or an analog thereof. Telaprevir is an oligopeptide consisting of N-(pyrazin-2-ylcarbonyl)cyclohexylalanyl, 3-methylvalyl, octahydrocyclopenta[c]pyrrole-1-carboxy, and 3-amino-N-cyclopropyl-2-oxohexanamide residues joined in sequence. Telaprevir has the chemical name (3S,3aS,6aR)-2-[(2S)-2-[[(2S)-2-cyclohexyl-2-(pyrazine-2-carbonylamino)acetyl]amino]-3,3-dimethylbutanoyl]-N-[(3S)-1-(cyclopropylamino)-1,2-dioxohexan-3-yl]-3,3a,4,5,6,6a-hexahydro-1H-cyclopenta[c]pyrrole-3-carboxamide. Telaprevir is marketed under the brand names INCIVEK and INCIVO for the treatment of hepatitis C co-developed by Vertex Pharmaceuticals and Johnson & Johnson.

In some embodiments, the compound is zafirlukast (also known as ACCOLATE). Zafirlukast is in a class of medications called leukotriene receptor antagonists (LTRAs). It works by blocking the action of certain natural substances that cause swelling and tightening of the airways.

In some embodiments, the compound that at least partially blocks the activity of a bacterial DnaK is a compound that interferes with expression (e.g., transcription or translation) of DnaK. In some embodiments, the compound is a nucleic acid, such as a complementary DNA or RNA molecule that hybridizes to at least of a portion of the DnaK RNA. In some embodiments, the compound is a peptide nucleic acid. In some embodiments, the compound is an antisense RNA.

The nucleic acid can be an RNA, a DNA, or a combination thereof. For example, the inhibitory nucleic acid that inhibits the expression of DnaK can be an antisense RNA or a ribozyme. The inhibitory RNA can be designed with the aid of a computer program specifically prepared therefor. As appropriate, the inhibitory nucleic acid can be delivered by any suitable means, such as in a vector, particles or bacterial viruses such as phages.

In some embodiments, the antagonist comprises a nucleic acid molecule that comprises a nucleotide sequence that binds to at least a portion of a nucleotide sequence of DnaK. The nucleic acid molecule can be of any length, so long as at least part of the molecule hybridizes sufficiently to DnaK nucleic acid such as mRNA. The nucleic acid molecule can bind to any region of DnaK mRNA. In some embodiments, the nucleic acid molecule binds to a particular domain of DnaK mRNA.

In some embodiments, the antagonist can comprise a DNA molecule, such as an antisense DNA molecule.

A target sequence on a target mRNA can be selected from a given cDNA sequence corresponding to DnaK, in some embodiments, beginning 50 to 100 nt downstream (i.e., in the 3′ direction) from the start codon. The target sequence can, however, be located in the 5′ or 3′ untranslated regions, or in the region nearby the start codon.

In one embodiment, the DnaK inhibitory agent comprises a nucleic acid molecule that comprises a nucleotide sequence that binds to at least a portion of a nucleotide sequence of DnaK mRNA. In some embodiments, the nucleic acid molecule is a DNA. In some embodiments, the nucleic acid molecule is an RNA.

In some embodiments, the composition comprises an anti-sense DNA. Anti-sense DNA binds with mRNA and prevents translation of the mRNA. The anti-sense DNA can be complementary to a portion of DnaK mRNA. In some embodiments, the anti-sense DNA is complementary to the entire reading frame of DnaK. In some embodiments, the antisense DNA is at least about 15 nucleotides, at least about 20 nucleotides, at least about 25 nucleotides, at least about 30 nucleotides, at least about 35 nucleotides, at least about 40 nucleotides, at least about 50 nucleotides, at least about 75 nucleotides, at least about 100 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, or at least about 1500 nucleotides.

In some embodiments, the composition comprises an anti-sense RNA. Anti-sense RNA binds with mRNA and prevents translation of the mRNA. The anti-sense RNA can be complementary to a portion of DnaK mRNA. In some embodiments, the anti-sense RNA is complementary to the entire reading frame of DnaK. In some embodiments, the antisense RNA is at least about 15 nucleotides, at least about 20 nucleotides, at least about 25 nucleotides, at least about 30 nucleotides, at least about 35 nucleotides, at least about 40 nucleotides, at least about 50 nucleotides, at least about 75 nucleotides, at least about 100 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, or at least about 1500 nucleotides.

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. In some embodiments, there is 100% sequence identity between the nucleic acid for use as an inhibitor and at least 15 contiguous nucleotides of the DnaK target sequence.

Strands or regions that are complementary may or may not be 100% complementary (“completely or fully complementary”). It is contemplated that sequences that are “complementary” include sequences that are at least 50% complementary, and may be at least 50%, 60%, 70%, 80%, or 90% complementary. In some embodiments, DNA or RNA generated from sequence based on one organism may be used in a different organism to inhibit expression of DnaK. It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

Transcription factors are regulatory proteins that bind to a specific DNA sequence (e.g., promoters and enhancers) and regulate transcription of an encoding DNA region. Thus, transcription factors can be used to modulate the expression of DnaK. Typically, a transcription factor comprises a binding domain that binds to DNA (a DNA-binding domain) and a regulatory domain that controls transcription. Where a regulatory domain activates transcription, that regulatory domain is designated an activation domain. Where that regulatory domain inhibits transcription, that regulatory domain is designated a repression domain. Thus, modulation of bacterial transcription factors can be used to reduce expression of DnaK.

In some embodiments, the transcription factor can be targeted with an antagonist of the invention, including anti-sense RNA or DNA to downregulate the transcription factor. Such antagonists can be identified by standard methods in the art, and in particular embodiments the antagonist is employed for treatment and or prevention of an individual in need thereof.

Antisense methodology takes advantage of the fact that nucleic acids tend to pair with complementary sequences. By complementary, it is meant that polynucleotides arc those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases, such as inosine, 5-methylcytosine, 6-methyladenine, hypoxanthine and others, in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, are employed to inhibit gene transcription or translation or both within a host cell, either in vitro or in vivo, such as within a host animal, including a human subject.

Ribozymes are RNA-protein complexes that cleave nucleic acids in a site-specific fashion. Ribozymes have specific catalytic domains that possess endonuclease activity. For example, a large number of ribozymes accelerate phosphoester transfer reactions with a high degree of specificity, often cleaving only one of several phosphoesters in an oligonucleotide substrate. This specificity has been attributed to the requirement that the substrate bind via specific base-pairing interactions to the internal guide sequence (“IGS”) of the ribozyme prior to chemical reaction. Ribozyme catalysis has primarily been observed as part of sequence specific cleavage/ligation reactions involving nucleic acids. For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression is particularly suited to the therapeutic applications. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Designing and testing ribozymes for efficient cleavage of a target RNA is a process well known to those skilled in the art. The identification of operative and preferred sequences for use in DnaK targeted ribozymes is simply a matter of preparing and testing a given sequence, and is a routinely practiced screening method known to those of skill in the art.

### Anticancer Therapy

The compound that at least partially blocks the activity of bacterial DnaK is able to increase the efficacy of an anticancer therapy. The anticancer therapy that is administered is not limiting. In some embodiments, the anti-cancer therapy comprises administration of an anti-cancer drug. In some embodiments, the anti-cancer drug includes but is not limited to oxaliplatin, cisplatin, docetaxel, etoposide, palbociclib, lenalidomide, and bortezamib.

In some embodiments, the anticancer therapy is selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and I 131 Iodine Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine) Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C). MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nivolumab, Nolvadex (Tamoxifen Citrate), Nplate (Romiplostim), Obinutuzumab, OEPA, Ofatumumab, OFF, Olaparib, Omacetaxine Mepesuccinate, Oncaspar (Pegaspargase), Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and I 131 Iodine Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

In some embodiments, the subject is administered one or more anticancer agents, surgery and/or radiotherapy in combination with the agent that inhibits DnaK.

In some embodiments, the anticancer agent is an immunotherapeutic agent. The cancer immunotherapy is not limiting and can include one or more immunotherapies. There are several different approaches to immunotherapy. For example, immunotherapies can include monoclonal antibodies, checkpoint inhibitors/immune modulators, therapeutic cancer vaccines, oncolytic viruses, adoptive T cell transfer, cytokines, and adjuvant immunotherapy.

The cancer that is being treated is not limiting. “Cancer” refers to leukemias, lymphomas, carcinomas, and other malignant tumors of potentially unlimited growth that can expand locally by invasion and potentially systemically by metastasis. Examples of cancers include, but are not limited to, cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

Prior to treatment, a sample from the patient's tumor can be subjected to one or more diagnostic/prognostic assays to detect bacteria (e.g., Mycoplasma, Mycoplasma fermentans, Heliobacter pylori, Fusobacterium nucleatum, Chlamydia trachomatis, or combinations thereof) or bacterial DnaK DNA, RNA, or protein from the aforementioned bacteria. In one nonlimiting example, RNA can be extracted from patients' cancer cells and reverse-transcribed into cDNA to detect and/or quantify DnaK levels using methods such as the polymerase chain reaction (e.g., RT-qPCR), as will be understood by those having ordinary skill in the art. DnaK protein levels from patients' cancer cells can be detecting using an immunologic method such as ELISA and/or can be quantified using methods such as mass spectroscopy. The correlation between DnaK levels and p53 mutation status can be assessed using well-known methods. Treatment as described herein can be administered to patients whose tumors are positive for DnaK expression and that possess functional p53.

### Bacterial DnaK

The bacterial DnaK that can be inhibited is not particularly limiting. In some embodiments, the bacterial DnaK is from M. fermentans, H. pylori, F. nucleatum and C. thrachomatis.

In some embodiments, the bacterial DnaK is encoded by a nucleic acid sequence from H. pylori described in NCBI Reference Sequence: NC_000915.1. In some embodiments, the bacterial DnaK is encoded by a nucleic acid sequence from F. nucleatum described in NCBI Reference Sequence: NP_603026.1.

### Screening Assays

In some embodiments, DnaK can be used in screening assays for compounds which bind and inhibit the interaction of DnaK with one or more of PARP1 or USP10. In some embodiments, the screening methods can be conducted in cells, cell-free preparations, cellular homogenates, animals, solution, or on one or more substrates, for example. In some embodiments any of a DnaK, PARP1, or USP10 or fragments or derivatives thereof, antibodies thereof, as well as test compounds can be used in the assay. In some embodiments, DnaK, or a fragment or derivative thereof is coupled to a solid surface, following by incubation with one or more of PARP1 or USP10, or fragments or derivatives thereof, and a test compound to assay for compounds that disrupt the interaction. In some embodiments, PARP1 or USP10, or fragments or derivatives thereof, are coupled to a solid surface, following by incubation with DnaK or fragments or derivatives thereof, and a test compound to assay for compounds that disrupt the interaction. In some embodiments, assays can be conducted that measure changes in mobility, e.g., on a gel or through a matrix, to assess competitive binding of a test compound to DnaK that disrupts an interaction with PARP1 or USP10. In some embodiments, the test compound can be labeled and contacted with DnaK, to assess binding thereto.

In some embodiments, screening assays can be performed in cells to assess the effect of DnaK on PARP1 activity, and screen for compounds that will inhibit the effect of DnaK on PARP1 activity. For example, cells such as HCT116 cells can be transfected with the DnaK expression vectors, treated with different DNA damaging agents (eg. H₂O₂, Etoposide, Topotecan, Bleomycin and actinomycin D) at different time points to induce PARP1 activity and then analyzed for PARPylation of appropriate proteins with a specific ELISA kit. The cells can be untreated or treated with a test compound and compared. In some embodiments, cells treated with DNA damaging agents can also be collected at different time points, and analyzed using the comet assay, a gel electrophoresis-based method that measures DNA damage in individual eukaryotic cells. In some embodiments, DNA repair can be monitored by incubating cells after treatment with damaging agent and measuring the damage remaining at selected intervals. In some embodiments, comet assays are performed under alkaline conditions to detect single-, double, or alkali-labile breaks.

In some embodiments, screening assays can be performed to assess the effects of DnaK binding on USP10 in the presence and absence of a test compound. In some embodiments, functional screening assays can be conducted in cells such as HCT116 cells. In some embodiments, the cells can be transfected with DnaK expression vectors, treated with DNA damaging agents (etoposide and low doses of actinomicyn D, both causing p53 activation), in the presence or absence of a test compound, and then analyzed for p53 stability and p53-dependent expression of p21, Bax and PUMA as described.

In some embodiments, screening assays can be performed to assess DnaK binding on USP10 in the presence and absence of a test compound by assaying ubiquitination of p53 (73) as provided herein.

In some embodiments, screening assays can be performed to assess whether one or more test compounds inhibit DnaK activation of one or more kinases. In some embodiments, the screening assays can be conducted in cells or cell free systems. In some embodiments, cells or samples can express DnaK or be treated with exogenous DnaK in the presence and absence of a test compound, and phosphorylation of one or more kinases can be assayed.

### Pharmaceutical Compositions

The DnaK antagonist can be administered in pharmaceutical compositions in a variety of ways and is not particularly limiting. In some embodiments, the agent is administered directly (topically), intravenously, subcutaneously, transcutaneously, intrathecally, intramuscularly, intracutaneously, intragastrically, intranasally, rectally, intra-arterially, parenterally, or orally.

In some embodiments, telaprevir is formulated and/or administered as described in U.S. Pat. No. 8,431,615, which is incorporated by reference herein.

In some embodiments, an effective amount of the antagonist of DnaK that is administered includes a dose of about 0.0001 nM to about 2000 μM. In some embodiments, amount administered is from about 0.01 nM to about 2000 μM; about 0.01 μM to about 0.05 μM; about 0.05 μM to about 1.0 μM; about 1.0 μM to about 1.5 μM; about 1.5 μM to about 2.0 μM; about 2.0 μM to about 3.0 μM; about 3.0 μM to about 4.0 μM; about 4.0 μM to about 5.0 μM; about 5.0 μM to about 10 μM; about 10 μM to about 50 μM; about 50 μM to about 100 μM; about 100 μM to about 200 μM; about 200 μM to about 300 μM; about 300 μM to about 500 μM; about 500 μM to about 1000 μM; about 1000 μM to about 1500 μM; and about 1500 μM to about 2000 μM. Of course, all of these amounts are exemplary, and any amount in-between these points is also expected to be of use in the invention.

In some embodiments, the antagonist can be administered parenterally or alimentarily. Parenteral administrations include, but are not limited to intravenously, intradermally, transdermally, intramuscularly, intraarterially, intrathecally, subcutaneous, or intraperitoncally. See, e.g., U.S. Pat. Nos. 6,613,308, 5,466,468, 5,543,158; 5,641,515; and 5,399,363 (each specifically incorporated herein by reference in its entirety). Alimentary administrations include, but are not limited to orally, buccally, rectally, or sublingually.

In some embodiments, the administration of the therapeutic compounds and/or the therapies of the present invention may include systemic, local and/or regional administrations, for example, topically (dermally, transdermally), via catheters, implantable pumps, dermal patches, transdermal patches, etc. Alternatively, other routes of administration are also contemplated such as, for example, arterial perfusion, intracavitary, intraperitoneal, intrapleural, intraventricular and/or intrathecal. The skilled artisan is aware of determining the appropriate administration route using standard methods and procedures. Other routes of administration are discussed elsewhere in the specification and are incorporated herein by reference.

As is well known in the art, a specific dose level of active compounds such as an antagonist of DnaK, or related-compounds thereof for any particular patient depends upon a variety of factors including the activity of the specific compound employed, the age, body weight, general health, sex, diet, time of administration, route of administration, rate of excretion, drug combination, and the severity of the particular disease undergoing therapy.

In some embodiments, the compound(s) or composition(s) can be administered to the subject once, such as by a single injection or deposition at or near the site of interest. In some embodiments, the compound(s) or composition(s) can be administered to a subject over a period of days, weeks, months or even years. In some embodiments, the compound(s) or composition(s) is administered at least once a day to a subject. Where a dosage regimen comprises multiple administrations, it is understood that the effective amount of the compound(s) or composition(s) administered to the subject can comprise the total amount of the compound(s) or composition(s) administered over the entire dosage regimen.

The present invention also contemplates therapeutic methods employing compositions comprising the active substances disclosed herein. Preferably, these compositions include pharmaceutical compositions comprising a therapeutically effective amount of one or more of the active compounds or substances along with a pharmaceutically acceptable carrier.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, α-tocopherol and the like; and the metal chelating agents such as citric acid, ethylenediamine tetraacetic acid (EDTA), sorbitol, tartaric acid, phosphoric acid and the like.

In some embodiments, the total daily dose of the active compounds of the present invention administered to a subject in single or in divided doses can be in amounts, for example, from 0.01 to 25 mg/kg body weight or more usually from 0.1 to 15 mg/kg body weight. Single dose compositions may contain such amounts or submultiples thereof to make up the daily dose. In general, treatment regimens according to the present invention comprise administration to a human or other mammal in need of such treatment from about 1 mg to about 1000 mg of the active substance(s) of this invention per day in multiple doses or in a single dose of from 1 mg, 5 mg, 10 mg, 100 mg, 500 mg or 1000 mg.

The active agents of the present invention can be administered alone or in combination with one or more active pharmaceutical agents. In some embodiments, the one or more active pharmaceutical agents are drugs that are useful for treating cancer.

Liquid dosage forms for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulation can be sterilized, for example, by filtration through a bacteria-retaining filter, or by incorporating sterilizing agents in the form of sterile solid compositions, which can be dissolved or dispersed in sterile water or other sterile injectable medium just prior to use.

In some embodiments, the active agents of the present invention can be administered as a nanoparticle formulation.

In order to prolong the effect of a drug, it is often desirable to slow the absorption of a drug from subcutaneous or intramuscular injection. The most common way to accomplish this is to inject a suspension of crystalline or amorphous material with poor water solubility. The rate of absorption of the drug becomes dependent on the rate of dissolution of the drug, which is, in turn, dependent on the physical state of the drug, for example, the crystal size and the crystalline form. Another approach to delaying absorption of a drug is to administer the drug as a solution or suspension in oil. Injectable depot forms can also be made by forming microcapsule matrices of drugs and biodegradable polymers, such as polylactide-polyglycoside. Depending on the ratio of drug to polymer and the composition of the polymer, the rate of drug release can be controlled. Examples of other biodegradable polymers include polyorthoesters and polyanhydrides. The depot injectables can also be made by entrapping the drug in liposomes or microemulsions, which are compatible with body tissues.

Suppositories for rectal administration of the drug can be prepared by mixing the drug with a suitable non-irritating excipient, such as cocoa butter and polyethylene glycol, which are solid at ordinary temperature but liquid at the rectal temperature and will, therefore, melt in the rectum and release the drug.

Solid dosage forms for oral administration may include capsules, tablets, pills, powders, gelcaps and granules. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferably, in a certain part of the intestinal tract, optionally in a delayed manner. Examples of embedding compositions which can be used include polymeric substances and waxes.

Dosage forms for topical or transdermal administration of a compound of this invention further include ointments, pastes, creams, lotions, gels, powders, solutions, sprays, inhalants or patches. Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In one embodiment, the therapeutic compound is delivered transdermally. The term “transdermal delivery” as used herein means administration of the pharmaceutical composition topically to the skin wherein the active ingredient or its pharmaceutically acceptable salts, will be percutaneously delivered in a therapeutically effective amount.

In some embodiments, the composition to be applied transdermally further comprises an absorption enhancer. The term “absorption enhancer” as used herein means a compound which enhance the percutaneous absorption of drugs. These substances are sometimes also referred to as skin-penetration enhancers, accelerants, adjuvants and sorption promoters. Various absorption enhancers are known to be useful in transdermal drug delivery. U.S. Pat. Nos. 5,230,897, 4,863,970, 4,722,941, and 4,931,283 disclose some representative absorption enhancers used in transdermal compositions and for topical administration. In some embodiments, the absorption enhancer is N-lauroyl sarcosine, sodium octyl sulfate, methyl laurate, isopropyl myristate, oleic acid, glyceryl oleate or sodium lauryl sulfoacetate, or a combination thereof. In some embodiments, the composition contains on a weight/volume (w/v) basis the absorption enhancer in an amount of about 1-20%, 1-15%, 1-10% or 1-5%. In some embodiments, to enhance further the ability of the therapeutic agent(s) to penetrate the skin or mucosa, the composition can also contain a surfactant, an azone-like compound, an alcohol, a fatty acid or ester, or an aliphatic thiol.

In one embodiment, the therapeutic compound is delivered via a transdermal patch.

In some embodiments, the invention provides a transdermal patch comprising an effective amount of the therapeutic compound for treating or preventing Alzheimer's disease. In some embodiments, the transdermal patch further comprises an absorption enhancer.

In some embodiments, the transdermal composition can further comprise one or more additional excipients. Suitable excipients include without limitation solubilizers (e.g., C₂-C₈ alcohols), moisturizers or humectants (e.g., glycerol [glycerin], propylene glycol, amino acids and derivatives thereof, polyamino acids and derivatives thereof, and pyrrolidone carboxylic acids and salts and derivatives thereof), surfactants (e.g., sodium laureth sulfate and sorbitan monolaurate), emulsifiers (e.g., cetyl alcohol and stearyl alcohol), thickeners (e.g., methyl cellulose, ethyl cellulose, hydroxymethyl cellulose, hydroxypropyl cellulose, polyvinylpyrrolidone, polyvinyl alcohol and acrylic polymers), and formulation bases or carriers (e.g., polyethylene glycol as an ointment base). As a non-limiting example, the base or carrier of the composition can contain ethanol, propylene glycol and polyethylene glycol (e.g., PEG 300), and optionally an aqueous liquid (e.g., isotonic phosphate-buffered saline).

In accordance with a particular embodiment of the present invention, compositions comprising at least one DnaK antagonist compound (as described above) and a pharmaceutically acceptable carrier are contemplated.

Exemplary pharmaceutically acceptable carriers include carriers suitable for oral, intravenous, intrathecal, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use. The active compound is admixed under sterile conditions with a pharmaceutically acceptable carrier and any needed preservatives or buffers as may be required.

The treatments may include various “unit doses.” Unit dose is defined as containing a predetermined quantity of the therapeutic composition (an antagonist of DnaK) calculated to produce the desired responses in association with its administration, e.g., the appropriate route and treatment regimen. The quantity to be administered, and the particular route and formulation, are within the skill of those in the clinical arts. Also of importance is the subject to be treated, in particular, the state of the subject and the protection desired. A unit dose need not be administered as a single injection but may comprise continuous infusion over a set period of time.

The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical compositions will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The compounds and compositions of the invention can be formulated into a composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions are administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the compounds and composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition can be combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of pharmaceutical lipid vehicle compositions that include compounds or compositions of the invention, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the therapeutics may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic and/or prophylactic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments of the present invention, the compounds and compositions of the invention are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like. See, e.g., U.S. Pat. Nos. 5,641,515; 5,580,579 and 5,792,451, each specifically incorporated herein by reference in its entirety. The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In further embodiments, the compounds and compositions of the invention can be administered via a parenteral route. As used herein, the term “parenteral” includes routes that bypass the alimentary tract. Specifically, the pharmaceutical compositions disclosed herein may be administered for example, but not limited to intravenously, intradermally, transdermally, intramuscularly, intraarterially, intraventricularly, intrathecally, subcutaneous, or intraperitoneally. See, e.g., U.S. Pat. Nos. 6,7537,514; 6,613,308; 5,466,468; 5,543,158; 5,641,515; and 5,399,363 (each specifically incorporated herein by reference in its entirety).

In some embodiments, the therapeutic compound is administered intrathecally. In some embodiments, the compound is administered intrathecally via an implantable pump. In one embodiment, the implantable pump comprises a SynchroMed™ II pump that stores and delivers medication into the intrathecal space (Medtronic).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, dimethyl sulfoxide (DMSO), polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, the invention provides a pharmaceutical composition formulated for administration as an infusion, comprising an effective amount of at least one compound that at least partially blocks the activity of a bacterial DnaK, in combination with a pharmaceutically acceptable carrier. The compound that blocks the activity of DnaK is not limiting and examples are described herein. In some embodiments, the at least one compound that at least partially blocks the activity of a bacterial DnaK is selected from the group consisting of telaprevir (also known as VX-950), zafirlukast, and combinations thereof. In some embodiments, the pharmaceutical composition further comprises an effective amount of an anticancer agent. The anticancer agent is not limiting, and examples are described herein. In some embodiments, the anticancer agent is selected from the group consisting of oxaliplatin, cisplatin, docetaxel, etoposide, palbociclib, lenalidomide, bortezamib, and combinations thereof. In some embodiments, the subject is administered the infusion of a period of hours, days, or weeks. In some embodiments, the composition is delivered intravenously via a port.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, in some embodiments, one dosage may be dissolved in 1 ml of isotonic NaCl solution and either added to 1000 ml of hypodermoclysis fluid or injected at the proposed site of infusion, (see for example, Remington's Pharmaceutical Sciences 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In other embodiments, the compounds and compositions of the invention may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch.” For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described, e.g., in U.S. Pat. Nos. 5,756,353 and 5,804,212 (each specifically incorporated herein by reference in its entirety). Likewise, the delivery of drugs using intranasal microparticle resins and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

All publications herein are incorporated by reference to the same extent as if each individual publication or patent application was specifically and individually indicated to be incorporated by reference.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLES

### Example 1. DnaK Inhibition to Enhance Anticancer Therapy

The inventors propose that a common bacterial protein, DnaK, causes resistance to anti-cancer drugs, and correlates the presence of certain DnaKs in the patients' cancer microbiome with their potential ability to alter responses to anti-cancer therapy.

DnaK sequences can be identified in the cancer-associated microbiome from patients with colon cancer or with stomach cancer (likely harboring F. nucleatum and H. pylori, respectively). RT-qPCR analysis of mRNA from patients' cancer cells can detect the presence of transcribed RNA encoding DnaKs and correlate their levels with responses to anti-cancer therapy. DnaKs most similar to Mycoplasma DnaK can be cloned and tested in in vitro.

### Example 2. Methods of Treating Cancer by Inhibiting Bacterial DnaK to Restore Activities of Anticancer Drugs

Studies of human microbiota show that some bacteria are associated with cancers. Definitive establishment of the causal correlation between Helicobacter pylori and gastric cancer provided the first demonstration that bacteria can cause cancer (Table 1).

TABLE 1
Bacteria associated with human cancers

| Bacterium | Cancer | Frequency | Invades cells | % positivity |
|---|---|---|---|---|
| H. pylori | Stomach | 5^{th} most common | Yes | High seroprevalence (>80%) in the general population. |
| | cancer | | | Carcinogenic role in cancer established. |
| F. nucleatum | Colorectal | 4^{th} most common | Yes | Present in the general population. |
| | Cancer | | | Increased amount (36% cancer vs 16% normal) |
| | (CRC) | | | Increased amount (69% cancer vs 24% normal) |
| | | | | 87% more in cancer cells vs adjacent tissue |
| C. thrachomatis | Cervical | 3^{rd} most common | Yes | Co-infection with HPV increases risk of cervical cancer |
| | cancer | in women | | |

Since then, studies of the human microbiome have elucidated an array of complex interactions between prokaryotes and their hosts. Recent examples of studies in human patients have highlighted associations between Fusobacterium nucleatum and colorectal cancer, Chlamydia irachomatis and cervical cancer and Mycoplasmas and prostate and colorectal cancer, as well as non-Hodgkin's lymphoma (NHL) in HIV-seropositive subjects, strongly supporting them as leading bacterial candidates with oncogenic properties. Consistent with this idea, in vivo experiments in several chemically induced or genetically deficient mouse models showed that germ-free conditions reduce colonic tumor formation. Additional evidence of bacterial involvement in carcinogenesis was provided by studies in animal models showing reduction of tumor load after antibiotic manipulation of gut microbiota. However, precise bacterial pathogen-cancer relationships remain largely elusive, although several bacteria, particularly those establishing persistent intra-cellular infections, can alter host cell cycles, affect apoptotic pathways, and stimulate the production of inflammatory substances linked to DNA damage, thus potentially promoting abnormal cell growth and transformation.

In vitro and in vivo studies demonstrate that Mycoplasmas promote cellular transformation. Though most Mycoplasma are extracellular, some invade eukaryotic cells and have been associated with some human cancers, including prostate cancer, oral cell carcinoma and non-Hodgkin's lymphoma (NHL) in HIV-seropositive subjects. Although their role remains unclear and controversial, it has been shown that infection with Mycoplasma fermentans subtype incognitus induces chromosomal alterations in vitro that result in phenotypic changes leading to acquisition of malignant properties in mouse and human cells, including loss of anchorage dependency, ability to form colonies in soft agar, and tumorigenicity in nude mice. Infections with several Mycoplasmas (fermentans, arginini, hominis and arthritidis) inhibit p53 activity and cooperate with Ras in oncogenic transformation in vitro, although the responsible bacterial protein has not been identified. Finally, it has also been shown that persistent infection with M. penetrans in a chemically immunosuppressed mouse model resulted in reduced p53 and p21 expression in gastric mucosal cells, leading to pathological changes. These findings indicate that, in some cases, Mycoplasmas could facilitate tumorigenesis, though (as mentioned above) no direct carcinogenic role for any Mycoplasmas has been demonstrated in vivo.

Importance of PARP1 in DNA repair. PARP (poly-ADP ribose polymerase)-1 is one of the most studied members of the family of PARP proteins. PARP1 is involved in the recognition and subsequent repair of single and double strand breaks in DNA. Following interaction with forms of damaged DNA, PARP1 activity is increased dramatically, resulting in PARylation of several proteins, including itself, histones, topoisomerase 1 (TOP1), DNA-dependent protein kinase (DNA-PK) and others, and in recruitment of single-strand break repair (SSBR)/base-excision repair (BER) factors to the damaged site. Failure to properly repair DNA damage usually results in apoptosis, thus avoiding accumulation of DNA damage that can ultimately lead to cellular transformation. Mice lacking PARP1 exhibit high levels of sister chromatid exchange, increased chromosomal aberrations, including fusions, breaks, and telomere shortening, and double-mutant DNA-PK/PARP1-deficient mice develop a high frequency of T-cell lymphomas.

Importance of USP10 in the regulation of p53 anti-cancer functions. P53 is a major tumor suppressor, often called “the guardian of the genome” for its multiple anti-oncogenic activities. By tightly coordinating cell cycle and apoptotic responses, p53 ensures that DNA damage is properly repaired, or that the damaged cell is removed upon engagement of the apoptotic pathway. P53 is mutated in about 50% of human cancers, and a mutated p53 allele can lead to Li-Fraumeni syndrome, characterized by development of several types of cancers. In animal models, p53−/− mice develop cancers (mainly lymphomas and sarcomas) with nearly 100% penetrance. Several proteins regulate p53, of which USP10 (ubiquitin carboxyl-terminal hydrolase protein-10) is one of the most important. By removing conjugated ubiquitin from target proteins, including p53, USP10 increases p53 stability in unstressed cells. This process is very important during DNA-damage response, in which USP10 translocates to the nucleus and deubiquitinates p53, stabilizing it and thus regulating its response to DNA damage. It is thus clear that reduction of USP10 activity can have profound negative consequences on the anti-cancer functions of p53.

Experimental results have linked components of the human microbiome to cancer, and a widespread and concerted scientific effort is ongoing to identify potentially responsible bacteria and characterize the molecular mechanism(s) of tumor promotion and progression. Several different bacteria have been associated with human cancers. While H. pylori so far is the only one with clear epidemiological data to support causality, studies of other bacteria including F. nucleatum, chlamydia and Mycoplasmas strongly support the idea that they too have oncogenic properties. Although it seems plausible that accumulation of DNA-damage and inhibition of p53-activities play a major role in driving transformation, molecular mechanisms whereby these bacteria dysregulate cellular pathways and eventually result in cellular transformation are still largely unclear. We have isolated and characterized a strain of human Mycoplasma that reliably induces lymphoma in a Severe Combined Immuno-Deficient (SCID) mouse model. We showed that Mycoplasma DnaK, a chaperone protein belonging to the HSP70 family, co-immunoprecipitates with both PARP1, a critical protein the pathways involved in recognition of DNA damage and repair, and USP10, a key p53 regulator. Phylogenetic amino acid analysis shows that some other bacteria associated with human cancers (including certain Mycoplasmas, H. pylori F. nucleatum and C. thrachomatis) have highly related DnaKs, suggesting a possible common mechanism of cellular transformation. Our data thus indicate that Mycoplasmas, and perhaps certain other bacteria with closely related DnaK, seem to have oncogenic activity mediated through inhibition of DNA repair and p53 functions.

We showed that a strain of human Mycoplasma is able to induce lymphoma in a Severe Combined Immuno-Deficient (SCID) mouse model, consistent with a previously described lymphomagenesis dependent upon reduced p53 activity. Our data demonstrate that interaction of Mycoplasma DnaK with PARP1 reduces its ability to promote DNA repair, and interactions of DnaK with USP10 reduce p53 activities. Altering the activities of these critical cellular pathways would thus greatly increase chances of cellular transformation following DNA damage. It is clearly of biological interest and potential therapeutic relevance to verify these findings in broader studies to understand the physical basis and the mechanism(s) responsible for reduced activities and levels of these critical cellular pathways.

Our studies are based on novel conceptual and scientific advances, including: i) the demonstration in vivo of tumorigenic properties of Mycoplasma; ii) the demonstration that a bacterial chaperone protein (DnaK) can interact with PARP1 and reduce its activities, thus likely resulting in accumulation of DNA damage, and can also interact with USP10, a key controller of p53, reducing its anti-cancer properties; and iii) the recognition that other cancer-associated bacteria carry closely related DnaKs, pointing to a potential common mechanism of carcinogenesis. Currently it is recognized that some bacteria are associated with cancer and may be involved in cellular transformation, but little is known about potential molecular mechanisms. An innovative aspect of our research is the presence of a unifying hypothesis that could explain how different bacteria could be linked in their ability to cause cellular transformation.

Test the hypothesis that DnaKs from different bacteria commonly associated with cancers bind to PARP1 and USP10, reducing PARP1 and p53 activities.
I. We recently demonstrated that DnaK, a member of the HSP70 chaperone family, from a strain of M. fermentans isolated in our laboratory from cells from an HIV-seropositive person, binds to human PARP1 and reduces its catalytic activity. PARP1 activates and recruits to the site of DNA damage important components of the DNA-repair complex. Moreover, this DnaK also binds human USP10 (ubiquitin carboxyl-terminal hydrolase 10), a regulator of p53 stability, resulting in reduced p53 anticancer functions. Phylogenetic amino acid analysis shows that other bacteria associated with human cancers (including certain Mycoplasmas, H. pylori, F. nucleatum and C. trachomatis) have closely related DnaKs, indicating a potential common mechanism leading to cellular transformation. Herein we present an experimental strategy to clone and compare anti-PARP1 and anti-p53 activities of these DnaKs to identify a common molecular mechanism whereby different bacteria can interfere with DNA repair pathways and p53-dependent anti-cancer functions.

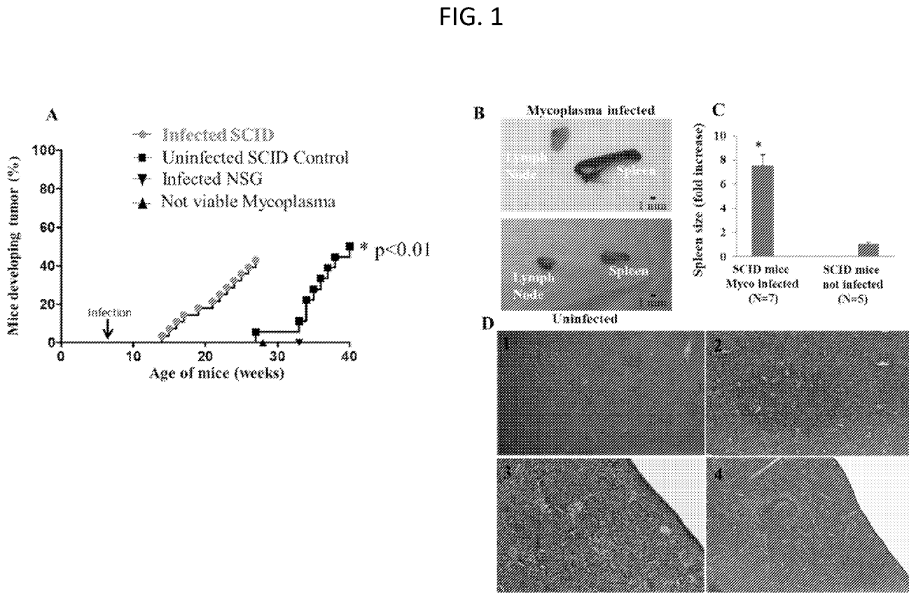

Our studies show that a strain of human Mycoplasma isolated and characterized in our laboratory reliably induces lymphoma in a Severe Combined Immuno-Deficient (SCID) mouse model, similar to previously described lymphomas resulting from reduced p53 activity (FIG. 1).

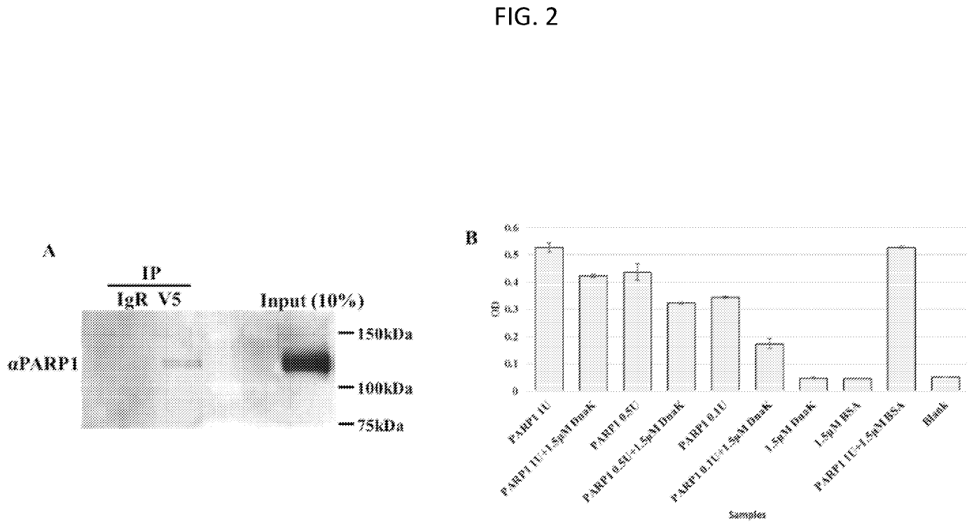

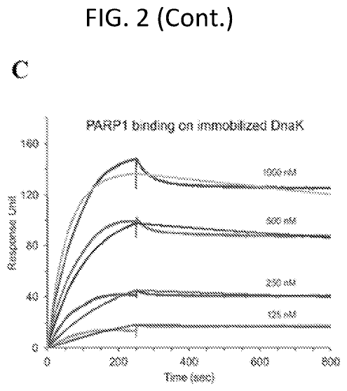

We also show that a Mycoplasma protein, DnaK, belonging to the HSP70 chaperone family, binds to human PARP1 and reduces its catalytic activity (FIGS. 2A, B and C). PARP1 activates important components of the DNA-repair complex and recruits them to the site of DNA damage.

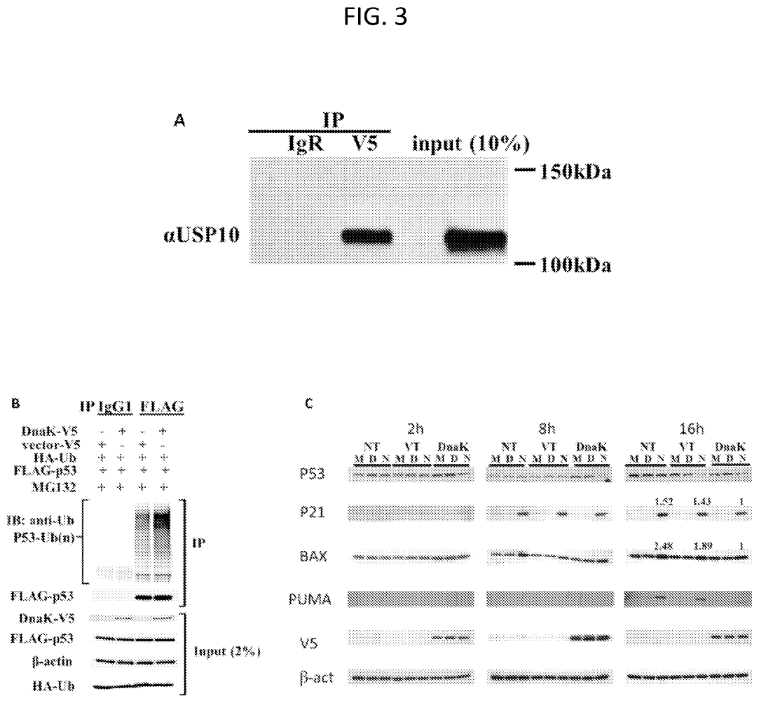

DnaK also binds human USP10 (ubiquitin carboxyl-terminal hydrolase 10), a regulator of p53 stability, (FIG. 3A) resulting in increased ubiquitination and reduced p53 stability (FIG. 3B). To analyze the effect of Mycoplasma DnaK on p53-dependent cellular pathways, HCT116 cells transfected with codon-optimized DnaK were treated with Nutlin-3, which releases active p53 from its natural ligand/inhibitor MDM2 (mouse double minute-2). The expression of p53, p21, Bax (Bcl-2-associated X protein), and PUMA (p53 up-regulated modulator of apoptosis) was analyzed for up to 16 h after transfection. Reduced levels of p21, Bax, and PUMA were observed when DnaK-transfected HCT116 cells were treated with Nutlin, as compared with control cells (FIG. 3C), indicating that Mycoplasma DnaK was impairing p53 functions. Of note, when the same experiments were performed with E. coli DnaK, we observed the opposite effect, i.e., an increase in p53 activity, as also previously reported by others. Taken together, our date indicate that in cells where DnaK is present, PARP1 and p53 activities are reduced, increasing the likelihood of DNA mutations, checkpoint failures, and consequent malignant transformation.

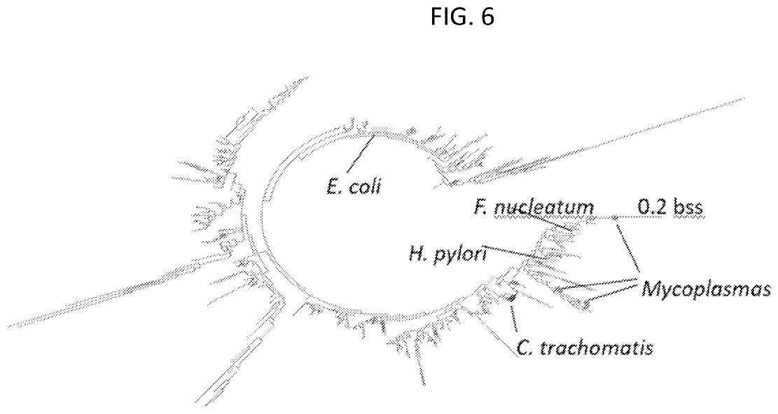

Given the potential oncogenic properties of Mycoplasma DnaK, we compared the DnaKs from known cancer-associated bacteria to see any sequence similarities that might potentially play a role in cellular transformation. Available amino acid sequences of DnaKs were aligned and the Mega 7.0.20 software used to create a phylogenetic tree (FIG. 6). We note that the Mycoplasma DnaK amino acid sequence is strikingly related to bacteria consistently associated with different types of human cancers, i.e., H. pylori, F. nucleatum and C. trachomatis. Conversely, these DnaKs are phylogenetically quite distinct from E. coli DnaK, which does not decrease p53 functions and has not been associated with cancer.

Studies to assess binding of different DnaKs with PARP1 and USP10. DNA encoding DnaKs of bacteria commonly associated with cancers will be synthesized and cloned into an expression vector as previously described. Strains that can be used are: H. pylori (NCBI Reference Sequence: NC_000915.1), F. nucleatum (NCBI Reference Sequence: NP_603026.1) and C. trachomatis (NCBI Reference Sequence: NC_000117.1). DNA sequences of the bacterial DnaKs (with a V5 Tag sequence in 3′) will be synthesized and cloned into expression vector as previously described. DnaK from E. coli can serve as negative control. Cloned DNA sequences can be verified by sequencing both DNA strands. DnaK expression vectors can be transfected into HCT116 cells to verify binding to PARP1 and USP10 by immunoprecipitation, as described (see also FIGS. 2A and 3A). For surface plasmon resonance (SPR) analysis of binding to PARP1 and USP10, the DnaK proteins can be expressed and purified as described. Once immobilized onto the CM5 biosensor chip, commercially available PARP1 and USP10 (expressed in baculovirus) can be quantified for binding to DnaK by SPR. In a preliminary SPR assay we have demonstrated very tight binding of PARP1 to immobilized DnaK (FIG. 2C), corroborating the immunoprecipitation results shown in FIG. 2A.

Studies to evaluate the effect of binding of DnaKs on PARP1 and on USP10 functions. To assess the effect of DnaK on PARP1 activity, HCT116 cells can be transfected with the DnaK expression vectors, treated with different DNA damaging agents (e.g., H₂O₂, Etoposide, Topotecan, Bleomycin and actinomycin D) at different time points to induce PARP1 activity and then analyzed for PARylation of appropriate proteins with a specific ELISA kit. Cells treated with DNA damaging agents can be collected at different time points, and analyzed using the comet assay, a gel electrophoresis-based method that measures DNA damage in individual eukaryotic cells. DNA repair can be monitored by incubating cells after treatment with damaging agent and measuring the damage remaining at selected intervals. Comet assays can be performed under alkaline conditions to detect single-, double, or alkali-labile breaks. Additionally, DNA damage induction by DNA damaging agents and cellular response upon transfection with the DnaK vectors can be monitored by Flow Cytometry analysis of histone H2AX phosphorylation in relation to cell cycle and apoptosis. DnaK proteins can be expressed and purified to assess effects on PARP1 enzymatic activity in vitro in an ELISA assay, as previously described. To assess the effects of DnaK binding on USP10, important regulator of p53 stability, HCT116 cells can be transfected with DnaK expression vectors, the cells can be treated with DNA damaging agents (etoposide and low doses of actinomicyn D, both causing p53 activation), then analyzed for p53 stability and p53-dependent expression of p21, Bax and PUMA as previously described.

Studies to map the domains involved in binding between PARP1 and DnaK. Deletion mutants of PARP1 and DnaK can be generated to express fragments representing different domains of the two proteins. For DnaK, these can include its substrate-binding domain (SBD) and the SBD in combination with the N-terminal actin-like nucleotide-binding domain (NBD), which modulates affinity for the substrate. For PARP1, constructs can be generated corresponding to the major functional domains of PARP1, including the DNA binding domain (DBD), the auto modification domain (AMD), the BRCT protein interaction domain (BRCT) and the WGR/catalytic domain (WGR/cat). These fragments can be used to conduct co-transfection studies followed by immunoprecipitation to map the domains involved in protein-protein interaction. Co-transfections can be used to confirm that the interactions measured by SPR result in reduced PARP1 biological activities, by using comet assays. This information allows identification of the interacting domains of DnaK and PARP1 and assess their contribution to interference with PARP1 activities.

Studies to verify through SPR analysis the specificity of the regions of interactions between PARP1 and DnaK. Following the identification of the domains interacting between PARP1 and DnaK, a more detailed analysis of the interactions can be performed. The DnaK domain can be expressed and purified as described. Subsequently, it can be immobilized on a chip and a series of peptides overlapping the domain of interest of PARP1 can be generated. Finally, these peptides can be used in competing binding experiments to map the specific area of interaction through SPR analyses. The reverse experiment can be to immobilize the PARP1 domain and perform the competing binding experiments with a series of peptides representing the binding domain of DnaK. The same experimental designs can be used to identify the regions of interactions between USP10 and DnaK.
II. It was previously shown that persistent infection with M. penetrans in a chemically immunosuppressed mouse model resulted in reduced p53 and p21 expression in gastric mucosal cells, leading to pathological changes. More recently, our data showed that Mycoplasma DnaK has broad potential tumorigenic properties in vivo and in vitro, by reducing the activities of cellular pathways critical for maintaining DNA integrity. To confirm the hypothesis that expression of this DnaK leads to tumor susceptibility and to directly study its functions in physiological processes, a transgenic (DnaK knock-in) mouse model with regulated expression of DnaK can be generated. The next step is to: i) monitor spontaneous tumor development, ii) assess susceptibility to chemically induced tumors, and iii) study DNA repair pathway responses of peripheral B- and T-cells to DNA-damaging agents. This provides proof of the oncogenic potential of these DnaKs as well as a useful in vivo model to more definitively characterize the underlying mechanisms.

We recently demonstrated that a strain of M. fermentans isolated in our laboratory from cells from an HIV-seropositive person induces lymphomas in a SCID mouse model. We also showed that Mycoplasma DnaK binds to human PARP1 and reduces its catalytic activity. PARP1 activates and recruits to the site of DNA damage important components of the DNA-repair complex. DnaK also binds to human USP10 (ubiquitin carboxyl-terminal hydrolase 10), a regulator of p53 stability, resulting in reduced p53 anti-cancer functions. Consequent to this binding, we observed reduced activity of PARP1 and p53 in vitro, and reduced effects of anti-cancer drugs that act through p53.

Test the hypothesis that cells from Mycoplasma DnaK knock-in mice have: i) higher rate of spontaneous tumor development, ii) are more susceptible to chemically induced tumors, and iii) have impaired PARP1 and p53 activities.

Studies to test for higher spontaneous tumor incidence in mice expressing DnaK. To study the effects of DnaK expression in vivo, an expression construct encoding M. fermentans DnaK can be inserted into the genome of C57BL/6NTac mice.

These can include conditional Tet-on and Tet-off constructs, in which DnaK expression is induced by the presence or absence of the tetracyclin analog doxycycline, respectively. A V5 epitope tag can be added to the 3′ end of the sequence, allowing for convenient monitoring of protein expression. In the Tet-off model, DnaK can be constitutively expressed in mice and its impact on the occurrence of spontaneous tumors can be evaluated. The animals can be closely monitored on a frequent basis for the occurrence of tumors or any unusual phenotype. To screen for the incidence of leukemia, mice can be bled every 3 months and subjected to complete blood count analyses. To determine spontaneous tumor occurrences, the animals in which DnaK is constitutively expressed can be routinely analyzed over their lifespan by visual inspection and palpation to evaluate the presence of solid tumor masses. Animals in each group can be evaluated for signs of spontaneous tumor development. Once a tumor is detected, the animals can be euthanized and tissues selected, placed in 10% buffered formalin, and submitted for histopathologic analyses. A total of 100 Tet-off animals can be followed over their life span, together with 100 Tet-on animals and 100 non-transgenic animals.

Studies to assess for increased susceptibility to non-hematopoietic cancers. A straightforward method to generate tumors in mice is to feed N-methyl-N-nitrosourea (MNU), an alkylating DNA-damaging compound that induces gastric cancers. To study increased susceptibility to gastric cancer following exposure to MNU, 4 week-old Tet-off DnaK knockin, Tet-on DnaK knockin and control animals (N=50 for each group) can be fed with 200 ppm MNU in drinking water twice a week for a total of 10 weeks, and at 50 weeks of age the animals can be sacrificed to determine gastric tumor incidence and histopathology, as described. The experiment can be repeated at least once to confirm results.

Studies to assess development, function and response to DNA-damaging agents of peripheral B- and T cells ex vivo. Because our Mycoplasma isolate causes lymphomas, it is important to look at the effects of its DnaK on lymphocytes. For the analysis of responses to DNA-damaging agents in B- and T-lymphocytes of Tet-off, Tet-on and non-transgenic control animals (N=10 for each group), cells can be ficoll-separated from peripheral blood lymphocytes and from spleens bead-purified, then treated with different DNA damaging agents (eg. H₂O₂, Etoposide, Topotecan, Bleomycin and actinomycin D) at different time points to induce PARP1 activity. Protein PARylation can be analyzed with a specific ELISA kit. In addition, we can also employ the comet assay to measure DNA damage in individual eukaryotic cells. Cells can be treated with the DNA damaging agent and incubated for different time intervals. The damage remaining at different time intervals can be analyzed to assess DNA repair. The comet assay can be performed under alkaline conditions to detect single-, double, or alkali-labile breaks. For analysis of p53 activities, cells can be treated with DNA damaging agents (etoposide and low doses of actinomicyn D, both causing p53 activation), then analyzed for p53 stability and p53-dependent expression of p21, Bax and PUMA as described. Additionally, DNA damage induction by DNA damaging agents and response in cells transfect with the DnaK vector can also be monitored by Flow Cytometry analysis of histone H2AX phosphorylation in relation to cell cycle and apoptosis. These assays can be performed using cells obtained from Tet-off and Tet-on animals (the latter with and without induction of in vivo expression of DnaK by doxycycline) and non-transgenic controls. At least three independent experiments with cells from animals of different ages (6 months, 1 year and 1.5 years) can be performed.

### Example 3. Mycoplasma Promotes Malignant Transformation In Vivo and its DnaK has Broad Oncogenic Properties

We provide evidence here that: i) a strain of Mycoplasma promotes lymphomagenesis in an in vivo mouse model; ii) a bacterial chaperone protein, DnaK, is likely implicated in the transformation process and resistance to anti-cancer drugs, by interfering with important pathways related to both DNA-damage control/repair and cell-cycle/apoptosis; and iii) the presence of a very low copy number of DNA sequences of Mycoplasma DnaK were found in some tumors of the infected mice. Other tumor-associated bacteria carry a similar DnaK protein. Our data suggest a common mechanism whereby bacteria can be involved in cellular transformation and resistance to anti-cancer drugs by a hit and hide/run mechanism.

We isolated a strain of human Mycoplasma that promotes lymphomagenesis in a Severe Combined Immuno-Deficient (SCID) mice, pointing to a p53-dependent mechanism, similar to lymphomagenesis in uninfected p53^{-/-} SCID mice. Additionally, Mycoplasma infection in vitro reduces p53 activity. Immuno-precipitation of p53 in Mycoplasma-infected cells identified several Mycoplasma proteins, including DnaK, a member of the Hsp70 chaperon family. We focused on DnaK because of its ability to interact with proteins. We demonstrate that Mycoplasma DnaK: i) interacts and reduces activities of human proteins involved in critical cellular pathways, including DNA-PK and PARP1, required for efficient DNA repair; and ii) binds to USP10 (a key p53 regulator), impairing p53-dependent anti-cancer functions. This also reduced efficacy of anti-cancer drugs that depends on p53 to exert their effect. Mycoplasma was detected early in the infected mice, but only low copy numbers of Mycoplasma DnaK DNA sequences were found in some primary and secondary tumors, pointing toward a “hit and run/hide” mechanism of transformation. Bystander, uninfected cells took up exogenous DnaK, suggesting a possible paracrine function in promoting malignant transformation, over and above cells infected with the Mycoplasma. Phylogenetic amino acid analysis shows that other bacteria associated with human cancers have similar DnaKs, consistent with a common mechanism of cellular transformation mediated through disruption of DNA-repair mechanisms, as well as p53 dysregulation that also results in cancer-drug resistance. This suggests that the oncogenic properties of certain bacteria is DnaK-mediated.

About 20% of human cancers are caused by known infectious agents (White et al., Clin Microbiol Rev, (2014), 27:463-481; Tagaya et al., Frontiers in Microbiology, (2017), 8:1425; Maman et al., Nature Reviews Cancer, (2018), 18:359-376. Some encode an oncogene (such as HTLV-1, HPV), transforming cells directly. Others, although not directly transforming, encode genes which interfere with cellular regulatory mechanisms, like the CagA protein of Helicobacter pylori, and the NS5A protein of HCV, both antagonizing the p53 pathway. In another mechanism the microbe does not infect the cell which becomes transformed, but alters the microenvironment so as to favor DNA damage or inappropriate survival of nearby cells (e.g. HIV-1 and again HCV and H. pylori) (Maman et al., Nature Reviews Cancer, (2018), 18:359-376; Buti et al., Proceedings of the National Academy of Sciences, (2011), 108:9238-9243; Kaplan-Turkoz et al., Proc Natl Acad Sci USA, (2012), 109:14640-14645; Majumder et al., Journal of Virology, (2001), 75:1401-1407; Lan et al., Oncogene, (2001), 21:4801; Lamb et al., J Cell Biochem, (2013), 114:491-497; Gallo et al., Proceedings of the National Academy of Sciences, (1999), 96:8324-8326; Lin et al., Annual Review of Pathology: Mechanisms of Disease, (2015), 10:345-370). In recent years, studies of the composition of the human microbiome and distribution of the microbiota elucidated an array of complex interactions between prokaryotes and their hosts (The Human Microbiome Project C, Nature, (2012), 486:207-214). A recent example is the association between Fusobacterium nucleatum and colorectal cancer (12-15) (Yu et al., Cell, (2017), 170:548-563; Bullman et al., Science, (2017), 358:1443-1448; Yang et al., Gastroenterology, (2017). 152:851-866; Purcell et al., Scientific Reports, (2017), 7:11590). However, precise bacterial pathogen-cancer relationships and the mechanisms involved inducing neoplasia remain largely elusive, though several bacteria, establishing persistent infections, can alter host cell cycles, affect apoptotic pathways, and stimulate the production of inflammatory substances linked to DNA damage, thus potentially promoting abnormal cell growth and transformation.

Some Mycoplasma are particularly suspicious bacteria for involvement in oncogenesis. Though most are extracellular, some invade eukaryotic cells and have been associated with some human cancers, including HIV-seropositive subjects with non-Hodgkin's lymphoma (NHL), prostate cancer and oral cell carcinoma (Yavlovich et al., Infection and Immunity, 72:5004-5011; Ainsworth et al., International Journal of STD & AIDS, 12:499-504; Barykova et al., Oncotarget, (2011), 2:289-297; Henrich et al., PLoS ONE, (2014), 9:e92297). In addition, it has been shown that persistent infection with Mycoplasma penetrans in a chemically immunosuppressed mouse model results in lower amount of p53 and p21 expression in gastric mucosal cells (Cao et al. et al., PLoS One, (2017), 12:e0180514). Moreover, in vitro infection of Mycoplasma fermentans subtype incognitus induces chromosomal alterations in both human prostate and murine embryonic cell lines, resulting in phenotypic changes leading to acquisition of malignant properties in mouse and human cells, including loss of anchorage dependency, ability to form colonies in soft agar and tumorigenicity in nude mice (Jiang et al., Journal of Cellular Biochemistry, (2008), 104:580-594; Namiki et al., PLoS ONE, (2009), 4:e6872; Zhang et al., BMC Cancer, (2006), 6:116-116). Finally, infections with several Mycoplasmas (fermentans, arginini, hominis and arthritidis) of different human cell lines (fibroblast, embryonic kidney, breast cancer, colorectal carcinoma) and mouse fibroblasts, inhibit p53 activity and cooperate with Ras in oncogenic transformation, though the responsible bacterial protein has not been identified (Logunov et al., Oncogene, (2008), 27:4521-4531). Although their role remains unclear and controversial, and to date no direct carcinogenic role for any Mycoplasma has been demonstrated in vivo, these findings are consistent with the notion that Mycoplasmas may facilitate tumorigenesis and in some cases be directly involved in one or more stages of their cause.

### Results

### Mycoplasma Induces Lymphoma In Vivo

Given the frequent detection of M. fermentans in HIV-1 seropositive subjects and its reported association with AIDS-related non-Hodgkin's lymphoma (NHL), we evaluated the tumorigenicity of this Mycoplasma in the context of immune deficiency (Ainsworth et al., International Journal of STD & AIDS, 12:499-504; Lo et al., The Lancet, (1991), 338:1415-1418). We used a strain of M. fermentans isolated in our laboratory from an HIV-1 positive cell line, about 0.5-1.5% different in nucleotide sequence from the Mycoplasma prototypes (SI Appendix, Materials and Methods and Fig. S1A-C). This Mycoplasma strain was used to infect a severe combined immune-deficient (SCID) mouse model. The SCID phenotype (Prkdc^{-/-}) is due to a defect in DNA repair caused by the lack of DNA-PK. B and T cells do not mature because of the inability to recombine immunoglobulin and T cell receptor chains, respectively (Bosma et al., Nature, (1983), 301:527). The inability to join dsDNA hampers the ability of these lymphocytes to progress through the cell cycle and eventually leads to their p53-dependent apoptosis (Kirchgessner et al., Science, (1995), 267:1178-1183; Gurley et al., Cancer Research, (1998), 58:3111-3115). Consequently, these animals lack B and T cells, although not completely, since some immature cells develop, particularly in the T cell lineage. Indeed, SCID^{prkdc-/-} mice develop T-cell lymphoma (about 40-60% within 32-48 weeks of age). SCID^{prkdc-/-} carrying an additional p53^{-/-} mutation develop T cell lymphomas at a faster rate (more than 90% by about 14 weeks of age), indicating that p53 provides a protective effect (Nacht et al., Genes & Development, (1996), 10:2055-2066). Given both the association of Mycoplasma with human tumors in vivo and the effect of Mycoplasma on p53 in vitro, we infected non-obese diabetic (NOD)/SCID and CB17.SCID mice with our isolates of M. fermentans to test the hypothesis that this Mycoplasma would accelerate lymphomagenesis by interacting with p53 in vivo (Logunov et al., Oncogene, (2008), 27:4521-4531). If this hypothesis was correct, we would expect that transformed T cells would appear soon after infection. As a negative control, we used NOD.Cg-Prkdc^{scid} Il2rg^{tm1Wjt}/SzJ mice, also known as NOD/SCID Gamma (NSG), which do not express the PRKDC gene or the X-linked IL-2R□ gene (Shultz L D, et al., The Journal of Immunology, (1995), 154:180-191). These animals very rarely develop spontaneous T-cell lymphoma even after sub-lethal irradiation, most likely because the lack of a functional IL-2 receptor further hampers T-cell proliferation. Uninfected controls and infected NSG mice did not develop tumors during the time of the experiment (FIG. 1A). However, following M. fermentans infection of the SCID mice, enlarged spleens, thymuses and lymph nodes were apparent as early as 8 weeks after infection (FIG. 1B,C). Histochemical analyses showed lymphoid cells infiltrating the organs of infected animals (FIG. 1D and SI Appendix, Fig. S2A-F). To verify that infiltrating lymphocytes causing organ enlargement were transformed, aliquots of single-cell suspensions from an enlarged lymph node of an M. fermentans infected animal were injected intra-peritoneally into young (about 6 weeks old) NOD/SCID mice. Extra-nodal tumors were detected as early as 2 weeks after injection. Secondary tumor cells were phenotypically characterized by flow cytometry. These cells were CD4⁺/CD8⁺ CD3^{high} and CD4⁺/CD8⁺ CD3, showing the same phenotype of the cells detected in uninfected mice developing spontaneous lymphomas at about 38-40 weeks of age (SI Appendix, Fig. S3A-C). Thus in Mycoplasma-infected animals, the tumor cells appeared much earlier during the life span of the animals, indicating the occurrence of a transforming event(s) soon after Mycoplasma infection (FIG. 1A). PCR analysis showed the presence of a very low copy number of Mycoplasma DNA sequences in enlarged spleens and lymph nodes of infected mice, and in secondary tumors composed of transformed cells originating from infected mice (SI Appendix, Table S1A-B).

Our data are consistent with an anticipated lymphomagenesis induced by reduction of p53 activity, similar to the one previously described in mice SCID^{prkdc-/-} Carrying an additional p53^{-/-} mutation (Nacht et al., Genes & Development, (1996), 10:2055-2066). Together with the presence in some primary and secondary tumors of Mycoplasma DNA sequences, it indicates that cellular transformation most likely originated through a “hit and hide/run” infectious process. Our data are also consistent with two previous reports: one, showing reduction of p53 and p21 in a chemically immunosuppressed mouse model infected by Mycoplasma, potentially facilitating malignant transformation, and the other, showing in vitro that infection of several rodent and human cell lines with M. fermentans, arginini, hominis, and arthritidis suppressed the transcriptional activity of p53 (Cao et al. et al., PLoS One, (2017), 12:e0180514; Logunov et al., Oncogene, (2008), 27:4521-4531). This impairment resulted in lack of transcription of p21, following treatment with 5-Fluorouracil (5-FU), a thymidilate synthase inhibitor that causes DNA damage and eventually results in activation of p53. Damaged cells proliferated and did not undergo apoptosis at the same rate as uninfected cells, raising the possibility that transforming events could accumulate in these cells (Logunov et al., Oncogene, (2008), 27:4521-4531). The Mycoplasma protein(s) responsible for the effect had not been identified.

Mycoplasma DnaK Binds USP10 and Impairs p53-Dependent Functions

To identify which M. fermentans protein is responsible for reducing p53 activities, pull-down experiments were conducted on Mycoplasma-infected HCT116 cells (colorectal carcinoma cell line) using an anti-p53 monoclonal antibody. Following infection, recovered products were characterized by HPLC mass-spectroscopy and micro-sequencing (SI Appendix, Table S2 and SI Appendix, Materials and Methods). Several Mycoplasma-specific proteins were identified, including DnaK, which is the prokaryotic heat shock protein Hsp70, a stress-induced protein. Eukaryotic organisms express several slightly different Hsp70 proteins when subjected to stressful conditions, and over-expression of some increases transformation of several human cell types (Jaattela et al., International Journal of Cancer, (1995), 60:689-693; Seo et al., Biochemical and Biophysical Research Communications, (1996), 218:582-587). Suppression of Hsp70 expression by anti-sense Hsp70 cDNA inhibits tumor cell proliferation and induces apoptosis (Kaur et al., International Journal of Cancer, (2000), 85:1-5).

While bacterial DnaK proteins form a family with diversity of amino acid sequences, they are a central hub in prokaryotic protein interaction networks (Calloni et al., Cell Rep, (2012), 1:251-264). For instance, DnaK from E. coli interacts with human and murine p53 (35-38), and increases p53 activity, though the meaning of these interactions is not clear (Clarke et al., Mol Cell Biol, (1998), 8:1206-1215; Nihei et al., Cancer Res, (19993), 53:1702-1705; Pinhasi-Kimhi et al., Nature, (1986), 320:182; Sturzbecher et al., Oncogene, (1987), 1:201-211; Hupp et al., Cell, (1992), 71:875-886).

To analyze the effect of Mycoplasma DnaK on p53-dependent cellular pathways, HCT116 cells transfected with codon optimized DnaK (SI Appendix, Fig. S4) were treated with Nutlin-3, which releases active p53 from its natural ligand and inhibitor, MDM2 (Mouse Double Minute) (Vassilev et al., Science, (2004), 303:844-848). Expression of p53, p21, Bax (Bcl-2 associated X protein) and PUMA (P53 Upregulated Modulator of Apoptosis) were then analyzed up to 16 hours after transfection. Reduced levels of p21, Bax and PUMA were observed when DnaK-transfected HCT116 cells were treated with Nutlin, as opposed to control cells (FIG. 2A), indicating that Mycoplasma DnaK was impairing p53 functions. Of note, when the same experiments were performed to investigate the effect of E. coli DnaK, we observed the opposite effects, i.e. increase in levels of p53 activities (SI Appendix, Fig. S5), as also previously reported by others (Hupp et al., Cell, (1992), 71:875-886).

P21 is a cyclin-dependent kinase inhibitor that is transcriptionally up-regulated by p53 in response to DNA damage, hypoxia, and nucleotide pool perturbation, leading to inhibition of retinoblastoma phosphorylation and cell cycle arrest at the G1 to S transition (Xiong et al., Nature, (1993), 366:701-704). We therefore investigated whether the previously observed reduced amounts of p53 and p21 (FIG. 2A) correlated with changes in the cell cycle. As expected, a marked increase in cells leaving G1 was observed in HCT cells treated with Nutlin and then transfected with Mycoplasma DnaK (FIG. 2B).

These data indicate that resistance to anti-cancers drugs that work at least in part by p53 activation may occur by infection with some Mycoplasmas. To test this hypothesis, we infected the cells treated with two drugs currently used in cancer treatment: 5-FU (5-Fluorouracyl) and Nutlin. As expected, Mycoplasma infection resulted in resistance to these anti-cancer drugs (FIG. 2C).

However, we failed to verify a direct interaction between p53 and transfected DnaK from M. fermentans. This suggests that DnaK may reduce p53 activity by binding to p53 with low affinity or that it binds to a regulatory protein(s) complex, which includes p53. Consequently, we determined the cellular proteins interacting with DnaK by performing pull down experiment of DnaK-transfected cells. Several proteins were identified (Table 1).

Among the proteins, USP10 (ubiquitin carboxyl-terminal hydrolase protein-10) is one of the most important regulators of p53. By removing conjugated ubiquitin from target proteins, including p53, USP10 increases p53 stability in unstressed cells. This process is very important during DNA-damage response, when USP10 translocates to the nucleus and deubiquitinates p53, stabilizing it and thus regulating its response to DNA-damage (Yuan et al., Cell, (2010), 140:384-396). We first confirmed interaction between DnaK and USP10 by immunoprecipitation studies (FIG. 3A). Next, we performed immunoblotting studies of cells treated with 5-FU and co-transfected with two vectors: one expressing USP10 and the other expressing DnaK. Presence of DnaK dramatically increased the amount of ubiquitinated p53 (FIG. 3B). This indicates that in the presence of DnaK p53 is less stable. Finally, to verify this effect on stability of p53, cells treated with 5-FU and cycloheximide, a protein-synthesis inhibitor, were transfected with DnaK, and the levels of p53 were measured over a short period of time (4 hours). The half-life of p53 was decreased in cells treated with 5-FU and transfected with DnaK, as opposed to the cells mock-transfected (FIG. 3C). Taken together our results indicate that DnaK binding to USP10 prevents its deubiquitinating activity, thus reducing p53 stability and its anti-cancer functions, and cellular response to some anti-cancer drugs.

Mycoplasma DnaK hampers activity of PARP1, a critical protein involved in DNA repair.

Another important protein listed in Table 1 is PARP (Poly-ADP ribose polymerase)-1, one of the most studied members of the family of PARP proteins. PARP1 is involved in the recognition and subsequent repair of DNA lesions (Godon et al., Nucleic Acids Res, (2008), 36:4454-4464; Schultz et al., Nucleic Acids Res, (2003), 31:4959-4964; Langelier et al., Science, (2012), 336:728-732). Following the interaction with damaged DNA, the activity of PARP1 is increased dramatically, resulting in PARylation of several proteins, including itself, histones, topoisomerase 1 (TOP1), DNA-dependent protein kinase (DNA-PK) and others (Ame et al., Bioessays, (2004), 26:882-893). This causes the recruitment to the damaged site of factors involved in double- and single-strand break repair (D/SSBR)/base-excision repair (BER) and nucleotide excision repair (NER) (Schreiber et al., Nat Rev Mol Cell Biol, (2006), 7:517-528; Pines et al., The Journal of Cell Biology, (2012), 199:235-249: Robu et al., Proceedings of the National Academy of Sciences, (2013), 110:1658-1663). Failure to properly repair DNA damages usually results in apoptosis to avoid accumulation of DNA damages that ultimately could lead to cellular transformation.

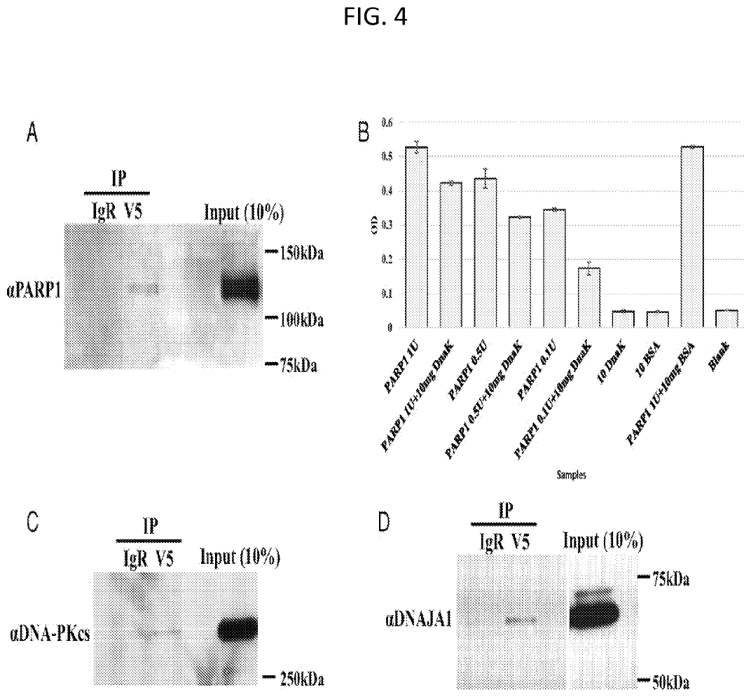

We first verified that DnaK could immunoprecipitate PARP1 (FIG. 4A). Next, we wanted to evaluate the effect of DnaK on the catalytic activity of PARP1. A colorimetric assay was used to measure the inhibitory effect of DnaK on PARP1 ability to PARylate histone immobilized on plates. A sharp decrease in histone PARylation was observed in the presence of DnaK, indicating that it hampered PARP1 catalytic activity (FIG. 4B). We also confirmed immunoprecipitation by DnaK of another protein important for DNA repair, i. e. DNA-PK (cs) (DNA-activated Protein Kinase, catalytic subunit) (FIG. 4C). Recruited to the site of damage by the heterodimer KU70/80 and forming a complex with other proteins, DNA-PK_{CS} is required for non-homologous end joining in both double strand DNA repair and V(D)J recombination (Ruscetti et al., Journal of Biological Chemistry, (1998), 273:14461-14467; Ying et al., Cancer Res, (2016), 76:1078-1088). For effective and proper functioning, the spatial and temporal arrangement of these important multi-proteins complexes must be very tightly controlled and regulated. The interaction of DnaK with two proteins important for the recognition of DNA damage and repair, resulting in decreased PARP1 catalytic activity, would likely lead to apoptosis or to accumulation of DNA damages, therefore increasing the probability of cellular transformation (Berwick et al., J Natl Cancer Inst, (2000), 92:874-897). Mice lacking PARP1 exhibit high levels of sister chromatid exchange, increased chromosome aberrations, including fusions, breaks, and telomere shortening, and double-mutant DNA-PK/PARP deficient mice develop high frequency of T-cell lymphomas (de Murcia et al., Proc Natl Acad Sci USA, (1997), 94:7303-7307; Wang et al., Genes Dev, (1997), 11:2347-2358; d'Adda di et al., Nat Genet, (1999), 23:76-80; Morrison et al., Nature Genetics, (1997), 17:479).

Chaperone activity of HSP70/DnaK is controlled by cycles of ATP binding and hydrolysis (Nunes et al., Nature Communications, (2015), 6:6307). Though DnaK itself is a weak ATPase, interaction with the co-chaperone DNAJ proteins (members of the HSP40 family) increases ATPase activity, promotes binding with target proteins and accelerates protein-folding activity of HSP70/DnaK (Clerico et al., J Mol Biol, (2015), 427:1575-1588). To determine whether intracellular Mycoplasma DnaK has possible chaperone activity, we verified its binding with a human DNAJ, previously identified in protein sequencing of DnaK-bound cellular proteins (Table 1). Immunoprecipitation studies confirmed that DnaK is able to bind human DNAJ1A1 (FIG. 4D). This could indeed indicate that, once in the intracellular compartments, bacterial DnaK becomes functionally active by exploiting the cellular co-chaperon DNAJA1. This suggests that DnaK negatively affects eukaryotic proteins by three possible mechanisms: i) direct binding and thus hampering their ability to form proper functional complexes; ii) direct binding and improperly folding of the target proteins, thus rendering them inactive and/or targeting them for degradation; and iii) binding to complex(es) of proteins, and altering their effectiveness.

### Exogenous Mycoplasma DnaK is Taken-Up by Bystander Cells

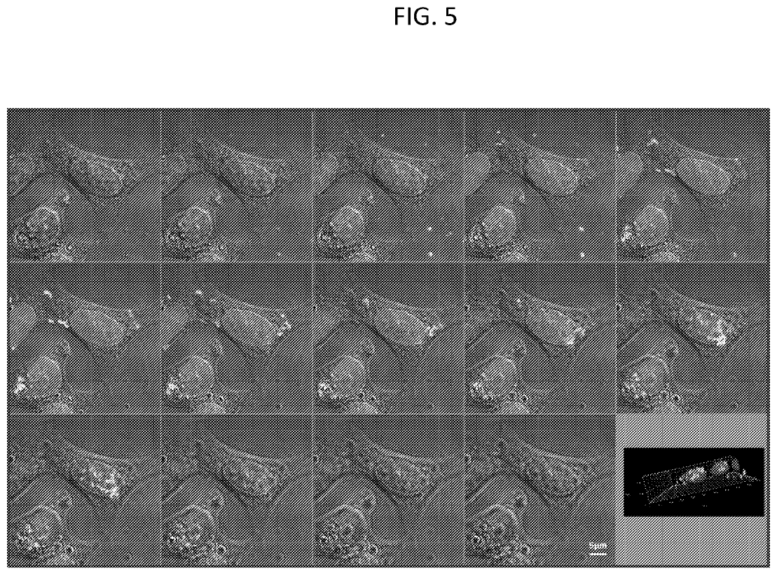

Bacteria can translocate proteins into eukaryotic cells by either attaching to the outside of the cellular membrane or by invading the cell (Costa et al., Nature Reviews Microbiology, (2015), 13:343; Holland I B et al., Biochimica et Biophysica Acta (BBA)—Molecular Cell Research, (2014), 1694:5-16). In addition, prokaryotic and eukaryotic membrane-localized HSP70 proteins may be released into the surrounding microenvironment, and then translocate into the cytoplasm of nearby cells (Carrio et al., J Bacteriol, (2005), 187:3599-3601; Vega et al., The Journal of Immunology, (2008), 180:4299-4307; Mambula et al., Methods, (2007), 43:168-175; Theriault et al., J Immunol, (2006), 177:8604-8611; Bendtsen et al., BMC Microbiology, (2005), 5:58). Given these properties of HSP70 proteins, we tested the ability of exogenous Mycoplasma DnaK to be taken-up by bystander cells. A recombinant protein (DnaK-V5) was constructed and added to HCT116 cells. After 24 hours exogenous Mycoplasma DnaK-V5 was localized in several cellular compartments, including cytoplasm, perinuclear membrane and nucleus (FIG. 5). These results expand our knowledge of previously published data on the ability of certain cells to bind and internalize HSP70s (Theriault et al., J Immunol, (2006), 177:8604-8611). The cellular uptake of DnaK-V5 was visualized using the Z-stacks option, where the gallery of images show clear presence of the protein inside the cells. The lower image in the right corner of the figure is a 3D presentation based on the collected Z-stacks of corresponding gallery of images (FIG. 5A-B). Two negative controls, that were imaged under the same conditions, are presented in FIG. 5. In conclusion, our data demonstrate that exogenous Mycoplasma DnaK is taken up by uninfected cells and this may result in impairment of pathways relevant for critical cellular functions and thereby alter control of cell growth in uninfected cells.

Amino Acid Analysis Reveals Similarities Among Bacterial DnaKs Associated with Human Cancers

Several bacteria have been associated with certain human cancers. The most notable is H. pylori and gastric cancer (Warren et al., Lancet, (1983), 1:1273-1275). Others are F. nucleatum, mainly associated with colorectal cancer (12-14), C. thracomatis, associated with cervical cancer, and some Mycoplasma associated with non-Hodgkin's lymphoma (NHL)(17), prostate cancer and oral cell carcinoma (Yu et al., Cell, (2017), 170:548-563; Bullman et al., Science, (2017), 358:1443-1448; Yang et al., Gastroenterology, (2017), 152:851-866; Ainsworth et al., International Journal of STD & AIDS, 12:499-504; Henrich et al., PLoS ONE, (2014), 9:e92297; Arnheim et al., Cancer Epidemiology Biomarkers &amp; Prevention, (2011), 20:2541-2550.; Smith et al., Int J Cancer, (2004), 111:431-439; Stone et al., Epidemiology, (1995), 6:409-414). The mechanisms of cellular transformation are largely unknown, though at least one has been proposed for H. pylori, whereby the CagA protein alters the p53 pathways (Buti et al., Proceedings of the National Academy of Sciences, (2011), 108:9238-924). We note that these bacteria have in common with Mycoplasma the ability to invade cells and, similarly to H. pylori, disseminate key proteins into the cellular cytoplasm and thus possibly transform the cell. Given the oncogenic properties of Mycoplasma DnaK, we compared the DnaKs from cancer-associated bacteria, to highlight any similarities that might potentially play a role in mechanisms of cellular transformation. Available amino acid sequences of DnaKs were aligned and the Mega 7.0.20 software used to create a phylogenetic tree (FIG. 6) (Kumar et al., Mol Biol Evol, (2006), 33:1870-1874). We note that Mycoplasma DnaK amino acid sequence is strikingly close to the ones of H. pylori, F. nucleatum and C. trachomatis, bacteria consistently associated with different types of human cancers. Conversely, all these DnaKs are phylogenetically distinct from E. coli DnaK, which does not decrease p53 functions (35-39) (FIG. 6) (Clarke et al., Mol Cell Biol, (1998), 8:1206-1215; Nihei et al., Cancer Res, (19993), 53:1702-1705; Pinhasi-Kimhi et al., Nature, (1986), 320:182; Sturzbecher et al., Oncogene, (1987), 1:201-211; Hupp et al., Cell, (1992), 71:875-886. It thus appears that other bacteria able to establish intracellular infection, and associated with cancers, carry a DnaK likely able to interact to a varying degree with cellular proteins implicated in critical cellular pathways, which can contribute to cellular transformation events and possibly reducing the effect of anti-cancer-drugs through the same mechanism(s).

A growing number of bacteria have been associated with human cancers. While H. pylori so far is the only bacterium with clear epidemiological data supporting a causal association and with a detailed molecular mechanism now proposed, studies of other bacteria including F. nucleatum, C. trachomatis and Mycoplasmas strongly support their role as leading candidates with oncogenic properties (Buti et al., Proceedings of the National Academy of Sciences, (2011), 108:9238-9243; Yu et al., Cell, (2017), 170:548-563; Bullman et al., Science, (2017), 358:1443-1448; Yang et al., Gastroenterology, (2017), 152:851-866; Ainsworth et al., International Journal of SID & AIDS, 12:499-504; Barykova et al., Oncotarget, (2011), 2:289-297; Henrich et al., PLoS ONE, (2014), 9:e92297; Cao et al. et al., PLoS One, (2017), 12:e0180514; Jiang et al., Journal of Cellular Biochemistry, (2008), 104:580-594; Namiki et al., PLoS ONE, (2009), 4:e6872; Zhang et al., BMC Cancer, (2006), 6:116-116; Warren et al., Lancet, (1983), 1:1273-1275; Arnheim et al., Cancer Epidemiology Biomarkers &amp; Prevention, (2011), 20:2541-2550; Smith et al., Int J Cancer, (2004), 111:431-439; Stone et al., Epidemiology, (1995), 6:409-414). While accumulation of DNA-damage and hampering p53-activity play a major role in driving transformation, the molecular mechanisms whereby these bacteria dysregulate cellular pathways are largely unknown.

We show here that a specific strain of Mycoplasma promotes lymphomagenesis in a murine in vivo model. These animals (Prkdc^{-/-}) have a defect in a DNA-repair gene, DNA-PK, and the mice ultimately develop spontaneous T-cell lymphoma (Bosma et al., Nature, (1983), 301:527; Kirchgessner et al., Science, (1995), 267:1178-1183; Gurley et al., Cancer Research, (1998), 58:3111-3115). Previous studies show that SCID^{prkdc-/-} with an additional p53^{-/-} mutation develop T cell lymphomas earlier, and that this model is suitable to detect oncogenic agents affecting DNA-repair and p53 activities (Gurley et al., Cancer Research, (1998), 58:3111-3115; Nacht et al., Genes & Development, (1996), 10:2055-2066). According to our data, Mycoplasma infection causes a series of events leading to cell transformation at a faster rate. Our data are in accordance with previous studies in vitro and in vivo which highlighted the oncogenic properties of Mycoplasma, though the precise molecular mechanism(s) has not been identified (Cao et al. et al., PLoS One, (2017), 12:e0180514; Jiang et al., Journal of Cellular Biochemistry, (2008), 104:580-594; Namiki et al., PLoS ONE, (2009), 4:e6872; Zhang et al., BMC Cancer, (2006), 6:116-116; Logunov et al., Oncogene, (2008), 27:4521-4531). We show here that DnaK, a bacterial chaperone protein belonging to the HSP70 family, interacts with several human proteins involved in important cellular pathways, namely USP10, PARP1 and DNA-PK_{CS}. Based on our data, we hypothesize that the presence inside the cell of bacterial DnaK protein interacting and hampering the function of cellular proteins critical for an effective DNA repair (PARP1 and DNA-PK_{C}) could lead to accumulation of DNA damages. At the same time, interaction of DnaK with USP10 reduces p53 activities, preventing its anti-cancer effectiveness. Reducing the efficacy of these two cellular pathways, which are critical for the detection, repair and prevention of DNA damages propagation, would greatly increase chances of cellular transformation following DNA breaks and chromosomal rearrangements caused by ionizing agents, chemicals and factors present in the microenvironment (Maman et al., Nature Reviews Cancer, (2018), 18:359-376). It would be of interest to study the possible interaction(s) of DnaK with components of the DNA mismatch repair system (MMR), since errors originating from spontaneous mutations constitute a great proportion of transformation events (Tomasetti et al., Science, (2017), 355:1330-1334).

A very low copy number of DNA sequence of Mycoplasma DnaK was also found in some of the primary and secondary tumor samples, pointing to a mechanism of “hit and hide/run” responsible for cellular transformation following bacterial infection. According to this hypothesis, once the cell is invaded, expression of DnaK would lead to cellular transformation (hit). At this point, a few copies of the bacterium DNA can be found in the tumor (hide), or not leave any trace of its presence (run). DnaK could exert these negative effects both in infected cells and in nearby uninfected cells, once this bacterial protein is released and subsequently taken up by these cells. We speculate that once in the cytoplasm, DnaK could then hamper a number of cellular pathways, perhaps even in the absence of continued bacterial infection. We also compared DnaK amino acid sequences among several bacteria frequently associated with human cancers. The similarities of these DnaKs suggest the possibility of a broad mechanism of tumorigenesis, which involves DnaK.

Our data may be clinically relevant for several reasons. First, several human cancers are due at least in part to events leading to DNA repair failures, a fact possibly heightened by the presence in the cell of certain DnaK proteins. This would indicate that the origin of cancers might involve bacteria more frequently than currently appreciated. Second, it is conceivable that DnaK of some bacteria could counteract the efficacy of compounds like 5FU or nutlin, used in the treatment of some cancers, which depend upon increased p53 activity for their activity. It is thus obviously of biological interest and potential therapeutic relevance to verify these findings in broader studies in humans and to understand the physical basis and the mechanism(s) responsible for reduced activities and levels of critical cellular pathways.

### Materials and Methods

### Animals

All animal experiments were approved by the University of Maryland School of Medicine IACUC. Female NOD/SCID and NOD/SCID gamma (NSG) mice were obtained from the Jackson Laboratory in Bar Harbor Maine. The mice are designated as Prkd scid/J. These mice carry several mutations that affect the immune system. Female CB17.SCID mice belonged to a colony maintained in our animal facility under pathogen-free conditions. A total of 10⁷ pfu/animal in 500 μl of PBS1× was injected in each animal of about 6 weeks of age (total of 30 animals) with Mycoplasma strain described in the Supplemental information. Aliquot of non-viable Mycoplasma was injected in 8 animals. Mycoplasma was heat-inactivated at 60° C. for 2 hours and non-viability was determined after retesting the same aliquots and verifying lack of growth. Both the CB17.SCID and the NOD/SCID mice develop thymic lymphomas at a very high rate (more than 40%) at around 8 months of age. We kept 18 of these animals as controls not infected to verify development of spontaneous lymphoma. As a further control, we injected 8 NSG mice, which rarely develop spontaneous lymphomas. All animals were kept in micro-Isolators caging systems under a controlled barrier system, to avoid any contamination. A control group was injected with sterile water and housed under the same conditions. At necropsy, all tissues were collected and placed in 10% formalin and later processed and stained with hematoxylin and eosin and were reviewed by a pathologist blindly.

At the end of 3-6 weeks we noted that the vast majority of the NOD/SCID animals ( 7/10) injected with PG18 suffered from pronounced weight loss and emaciation. Necropsy examination of the animals showed severe wasting and some mottling of the kidney. On histological examination there was an acute to chronic inflammation in the lungs, kidney, liver, and joints. Some animals displayed little to no sickness until in around 12 weeks of age at which they displayed some loss of weight, and some slow development of dyspnea (difficult breathing). On gross examination, in some animals the thymus area was enlarged with a tumor-like homogenous mass taking up a large portion of the chest cavity, and there was enlargement of the spleen, liver, and lymph nodes in the mesentery and peripheral areas. Histologically, the tumor mass was a homogenous lymphoblastic infiltration with highly mitotic figures (see FIG. 1D). Tumor invasion included the spleen, lymph nodes, kidneys and brain. There was no tumor development in the NSG mice or the control mice. Also the 8 animals injected with aliquots of non-viable Mycoplasma failed to develop tumors within 28 weeks of age.

### Western Blot Analysis

For western blot analysis, cell monolayers were detached by scraping, washed in cold PBS and solubilized in Ripa lysis buffer (Sigma) in the presence of protease inhibitors (Sigma). The amount of extracted protein was measured by the Bradford assay (Bio-Rad). 30 μg of proteins were resolved by SDS-page, transferred to polyvinylidene difluoride membrane (Bio-Rad), and probed with anti-p53 (Santa Cruz), anti-p21(Abcam), anti-Bax (Cell Signaling), anti-PUMA (Calbiochem), anti-V5 (Invitrogen), and anti-beta actin (Cell Signaling) antibodies. Blots were incubated with a secondary horseradish peroxidase (HRP)-conjugated antibody (Santa Cruz), developed using an ECL chemiluminescent substrate kit (Amersham Bioscience) and exposed to Kodak x-ray film.

### Immunoprecipitation

Immunoprecipitation experiments followed the protocol described in, with minor modifications. Detailed protocol and reagents are provided in the Supplemental Materials and Methods section.

### HPLC Analysis and Sequencing of Proteins

Following immunoprecipitation, the gel pieces from the band (each cut into 3 slices) were transferred to a siliconized tube and washed in 200 μL 50% methanol. The gel pieces were dehydrated in acetonitrile, rehydrated in 30 μL of 10 mM dithiolthreitol in 0.1 M ammonium bicarbonate and reduced at room temperature for 0.5 h. The DTT solution was removed and the sample alkylated in 30 μL 50 mM iodoacetamide in 0.1 M ammonium bicarbonate at room temperature for 0.5 h. The reagent was removed and the gel pieces dehydrated in 100 μL acetonitrile. The acetonitrile was removed and the gel pieces rehydrated in 100 μL 0.1 M ammonium bicarbonate. The pieces were dehydrated in 100 μL acetonitrile, the acetonitrile removed and the pieces completely dried by vacuum centrifugation. The gel pieces were rehydrated in 20 ng/μL trypsin in 50 mM ammonium bicarbonate on ice for 30 min. Any excess enzyme solution was removed and 20 μL 50 mM ammonium bicarbonate added. The sample was digested overnight at 37° C. and the peptides formed extracted from the polyacrylamide in a 100 μL aliquot of 50% acetonitrile/5% formic acid. This extract was evaporated to 15 μL for MS analysis.

The LC-MS system consisted of a Thermo Electron Velos Orbitrap ETD mass spectrometer system with an Easy Spray ion source connected to a Thermo 3 μm C18 Easy Spray column (through pre-column). 7 μL of the extract was injected and the peptides eluted from the column by an acetonitrile/0.1 M acetic acid gradient at a flow rate of 0.25 μL/min over 1.6 hours (3 bands per sample). The nanospray ion source was operated at 1.9 kV. The digest was analyzed using the rapid switching capability of the instrument acquiring a full scan mass spectrum to determine peptide molecular weights followed by product ion spectra (20) to determine amino acid sequence in sequential scans. This mode of analysis produces approximately 90000 MS/MS spectra of ions ranging in abundance over several orders of magnitude. Not all MS/MS spectra are derived from peptides. The data were analyzed by database searching using the Sequest search algorithm.

### Cell Culture Experiments and Cell Viability Assay

HCT116 cells (colon carcinoma cell line) were obtained from ATCC. Cells were maintained in McCoy's 5A medium (Invitrogen), supplemented with 10% fetal bovine serum. For the cell viability assay HCT116 Mycoplasma infected cells or transfected cells were plated in 96 well plates at a density of 15,000 cells/cm². Treatments were performed on the same day of plating and cells were harvested following 48 hours. The LIVE/DEAD® Viability/Cytotoxicity Kit (Invitrogen) was used to determine cell viability, following the manufacturer's instructions. In all experiments cell viability was calculated as a percentage relative to the control cultures. For infection experiments, HCT116 cells were infected with MF-I1 grown in aerobic condition, 10⁶ pfu/10⁶ cells. After 48 hours cells were plated in 96 well plates at a density of 5000 cells/well for the cell viability assay or in 75 cm² at a density of 150,000 cells/cm² for protein analysis. On the day of plating, cells were treated with 20 μM 5FU or 10 μM Nutlin-3 or a corresponding volume of DMSO as control. In some experiments cells were treated with 10 μM 5FU or 5 μM Nutlin-3. Cells were harvested after 48 hours for cell viability assay and after 16 hours for protein assays. For experiments with Mycoplasma DnaK, semi-confluent cell monolayers were first transfected with DnaK or with the vector control and then plated at the density described above. For time course experiments, transfected cells, vector control and no transfected control cells were plated at a density of 150.000 cells/cm2, treated with 2 μM Nutlin-3 or DMSO v/V and collected after 2 h, 8 h, 16 h and 24 h.

### Transfection

HCT116 cells were transiently transfected with the plasmid DNA using Lipofectamine 2000 (Invitrogen, Carlsbad, CA) following the manufacturer's protocol. Briefly, 25 μg plasmid DNA containing the insert or without the insert (control) was added to Lipofectamine suspended in reduced serum medium (OptiMEM from Invitrogen, Carlsbad, CA) and added to sub-confluent cultures of HCT116 p53^{+/+} and HCT116 p53^{-/-}, then incubated overnight at 37° C. in the presence of OptiMEM medium. Transfected cells were trypsinized and re-plated for subsequent experiments.

### Cell Cycle Analysis

Transfected HTC116 cells were plated in 6-well culture plates in the presence of serum-free McCoy's medium and incubated at 37° C. to allow cell cycle synchronization. Following overnight serum starvation, serum was added to the cells at a final concentration of 5% V/v with or without treatment with different concentrations (100, 10 and 1 μM) of Nutlin-3 (Sigma, St Louis, MO) or control DMSO and incubated for 0, 2, 8, 16 or 24 hours. Following incubation, cells were collected by trypsinization, washed with ice-cold phosphate buffered saline and used for staining with propidium iodide using the protocol described previously, with minor modifications (Romerio et al., FASEB J, (2002), 16:1680-1682). Briefly, washed cells were fixed with ice-cold 70% ethanol overnight at 4° C. Fixed cells were washed again and re-suspended in PBS containing 10 μg/ml propidium iodide (Sigma, St Louis, MO) and 20 μg/ml bovine RNase A (Roche Applied Sciences, Indianapolis, IN) in a 37° C. water bath for 45 minutes and analyzed by flow cytometry. The cell cycle status of cells was analyzed using the Flojo software (Flojo, Ashland, OR).

### Elisa Based Assay for Detection of PARP1 Activity

The ability of DnaK to inhibit PARP1 enzyme activity was assessed using Trevigen's HT Universal Colorimetric PARP1 Assay Kit, following manufacturer's instruction. Different concentration of PARP1 were incubated with 10 ug of DnaK-V5 protein as indicated, for 30 minutes in ice. The same units of PARP1 without DnaK-V5, and the highest amount of PARP1 was used with 10 μg of BSA as negative controls. A sample without enzyme was used as black control. The samples were then loaded in duplicate into a 96-well plate histone-coated, and incubated in the presence of biotinylated NAD and activated DNA for 1 hour at 37° C. The wells were then incubated first with Strep-HRP for 1 hour at room temperature and then with a colorimetric substrate, following 2 washes with 1×PBS+0.1% Triton X-100 and 2 washes with 1×PBS. Finally, the absorbance was measured with a 96-well plate reader with 450 nm filter.

### In Vitro Ubiquitination Assay

Ubiquitination of p53 was detected as described previously (Li et al., Nature, (2002), 416:648-653). Briefly, HCT116 cells were transiently transfected with Dnak-v5 or control vector, Flag-p53, and HA-ubiquitin expression plasmids. After 48 h the cells were treated for 5 h with 20 μM MG132 (Millipore), and were then lysed under non-denaturating conditions (Cell Signaling). Ubiquitin aldehyde (R&D) was added to the lysate to a final concentration of 1 μM. Lysates were pre-cleared with 50 μl of protein G Dynabeads (ThermoFisher) for 1 h at 4° C. with a rotator at 20 rpm. Anti-flag antibody (Sigma Aldrich) was used to immunoprecipitate ubiquitinated p53 proteins and mouse IgG1 (Sigma) was used as a control. Immunoprecipitated samples were resolved by SDS-polyacrylamide gel electrophoresis (12% gel from Novex) and analyzed by western blotting with anti-HA and anti-flag (both from Sigma). To ensure correct protein expression and loading, input samples were immunoblotted with anti-v5 (Abcam), anti-Flag, anti-R-actin (Cell Signaling) and anti-HA. pcDNA3 flag p53 was obtained from Thomas Roberts (Addgene plasmid #10838); pRK5-HA-Ubiquitin-WT was obtained from Ted Dawson (Addgene plasmid #17608).

### Cell Culturing and Immunofluorescent-Labelling for Detection of DnaK-V5 Cellular Uptake

For the immunofluorescence analysis, samples were prepared as follows. HCT116 cells (1×10⁴ cell/well cultured in McCoy media supplemented with 10% FBS, L-glutamine 1%, penicillin/streptomycin 1%), were seeded in a 4 well chambered coverglass (ThermoFisher Scientific) polylisin-coated, and treated with DnaK-V5 protein (80 μg/ml) for 24 hrs. Negative controls were not treated with DnaK-V5. After washing, cells were then fixed with 4% paraformaldehyde for 15 min at 37° C., washed with PBS1× and permeabilized with 0.1% Triton X-100 in 1×PBS for 15 min at RT, washed again and then blocked with 1% BSA and 10% serum from the specie the secondary antibody was raised in (normal goat serum), in 1×PBS for 60 min at RT. Primary labelling used a mouse monoclonal antibody directed against the V5 tag of the recombinant DnaK protein from M. fermentans. Cells were incubated in a humid chamber at RT with a 1:200 dilution of the primary antibody anti-Mycoplasma—DnaK-V5 (V5 Tag mouse monoclonal antibody ThermoFisher Scientific) for 2 hours. After 3 washes in PBS, cells were then incubated with 1:1000 dilution of fluorescent dye-labeled secondary antibody (goat anti-Mouse IgG FITC ThermoFisher Scientific) for 45 minutes at RT in the dark. Finally, cells were washed 3 times in PBS and PBS was added before immunofluorescence analysis. To demonstrate antibody specificity primary mouse Isotype control monoclonal antibody (ThermoFisher Scientific) and IgG fluorescein-conjugated secondary antibody were used as negative control (NCtrl). DAPI staining was used for nuclei detection (Sigma).

### PCR Analysis

Tissues were disrupted and homogenized using a rotor-stator homogenizer and total DNA was extracted with the DNeasy Blood & Tissue Kit (Qiagen). 50 ng of DNA were subjected to real time PCR using the iQ™ SYBR® Green Supermix Kit (Bio-Rad) with the ABI PRISM 5700 sequence detection system. All reactions were run in triplicate. Primers were selected using the NCBI/primer-blast program (www.ncbi.nlm.nih.gov/tools/primer-blast/) and were synthesized by Sigma-Aldrich: IS (PCR was performed with the following protocol: incubation at 95° C. for 5 minutes then 35 cycles of 30 sec at 95° C., 30 sec at 60° C., and 45 sec at 72° C.): forward, 5′-TCCCTTTCTTGACATGCTTTG-3′ (SEQ ID NO:3) and reverse, 5′-CGCCTAATTTAAGAATGGTTGG-3′ (SEQ ID NO:4) yielding a PCR product of 167 bp; DnaK 368-462 (PCR was performed with the following protocol: incubation at 95° C. for 5 minutes then 35 cycles of 30 sec at 95° C., 30 sec at 69° C., and 30 sec at 72° C.: forward 5′-ACAATGCACAACGTGAAGCCACA-3′ (SEQ ID NO:5) and reverse 5′-TGCTAAAGCAGCAGCAGTAGGTTCG-3′ (SEQ ID NO:6) yielding a PCR product of 94 bp; DnaK 367-716 (PCR was performed with the following protocol: incubation at 95° C. for 5 minutes then 35 cycles of 30 sec at 95° C., 30 sec at 62° C., and 45 sec at 72° C.): forward 5′-GACAATGCACAACGTGAAGC-3′ (SEQ ID NO:7) and reverse 5′-TCAGCAGCAGCTTTTAGACG-3′ (SEQ ID NO:8) yielding a PCR product of 350 bp; DnaK 367-954 (PCR was performed with the following protocol: incubation at 95° C. for 5 minutes then 35 cycles of 30 sec at 95° C., 30 sec at 62° C., and 45 sec at 72° C.): forward 5′-GACAATGCACAACGTGAAGC-3′ (SEQ ID NO:9) and reverse 5′-ACGTGTTGAACCACCAACAA-3′ (SEQ ID NO:10) yielding a PCR product of 587 bp; Dnak 688-1069 (PCR was performed with the following protocol: incubation at 95° C. for 5 minutes then 35 cycles of 30 sec at 95° C., 30 sec at 62° C., and 30 sec at 72° C.): forward 5′-GCAATGGCTCGTCTAAAAGC-3′ (SEQ ID NO:11) and reverse 5′-CTGCAAGAACAGCACCTTGA-3′ (SEQ ID NO:12) yielding a product of 381 bp; DnaK 1037-1508 (PCR was performed with the following protocol: incubation at 95° C. for 5 minutes then 35 cycles of 30 sec at 95° C., 30 sec at 70° C., and 30 sec at 72° C.): forward 5′-TGGGTGCTGCAATTCAAGGTGC-3′ (SEQ ID NO:13) and reverse 5′-GCACGTTTTGCATCAGCTTCACG-3′ (SEQ ID NO:14) yielding a product of 471 bp; R1 (123) (PCR was performed with the following protocol: incubation at 95° C. for 5 minutes then 35 cycles of 30 sec at 95° C., 30 sec at 61° C., and 30 sec at 72° C.): forward 5′-TCGCAACTCTAGATGCAGGAT-3′ (SEQ ID NO:15) and reverse 5′-AAACGAGTTGCTTGTTCTGCT-3′ (SEQ ID NO:16) yielding a product of 106 bp; R2 (1164) (PCR was performed with the following protocol: incubation at 95° C. for 5 minutes then 35 cycles of 30 sec at 94° C., 30 sec at 65° C., and 45 sec at 72° C.): forward 5′-ACGGCTTTTCCGTTTTTGTCTT-3′ (SEQ ID NO:17) and reverse 5′-TGCATCCATGAACCGTATCCA-3′ (SEQ ID NO:18) yielding a product of 106 bp; R3 (100) (PCR was performed with the following protocol: incubation at 95° C. for 5 minutes then 35 cycles of 30 sec at 94° C., 30 sec at 61° C., and 45 sec at 72° C.): forward 5′-AGCAATGGCTTTTGGTGATGC-3′ (SEQ ID NO:19) and reverse 5′-TGCATTGGACAGGCAAACGA-3′ (SEQ ID NO:20) yielding a product of 532 bp; R4 (95) (PCR was performed with the following protocol: incubation at 95° C. for 5 minutes then 35 cycles of 30 sec at 94° C., 30 sec at 61.5° C., and 45 sec at 72° C.): forward 5′-AGATGGGACATTAGACGGGA-3′ (SEQ ID NO:21) and reverse 5′-TCGCGAGGACTTACCAACAT-3′ (SEQ ID NO:22) yielding a product of 816 bp. PCR was performed with the following protocol: incubation at 95° C. for 5 minutes then 35 cycles of 30 see at 94° C., 30 see at 60° C., and 45 see at 72° C.

For cloning, PCR was performed with the same set of primers and conditions and the number of cycles was increased to 41. Bands were cloned into the PCRII TOPO plasmid (ThermoFisher Scientific), according to the manufacturer's protocol and sequenced to confirm identity with the targeted Mycoplasma sequence.

### Statistical Analysis

Time to developing lymphomas was performed using inverted Kaplan-Meier (KM) estimates with log-rank test. At-risk time for KM was calculated based on follow-up of 20 weeks after injection; mice that died were censored at the time of death. Differences in the proportions or percentages were tested using Fisher's exact test. Differences in the means were tested using Student t-test. All statistical tests were two-sided. Poisson regression was used to calculate statistical significance in FIG. 2C.

### Example 4. Method(s) to Inhibit DnaK with Telaprevir in Order to Restore Activities of Certain Anti-Cancer Drugs, Including Cisplatin and 5FU

It is shown herein that combinations of Telaprevir plus cisplatin and/or other anti-cancer drugs (like 5FU) can be used to treat cancers (like colon carcinoma, gastric carcinoma or esophageal carcinoma) where the presence of bacteria like Mycoplasmas and/or F. nucleatum can lead to drug resistance through a mechanism DnaK-dependent. The presence of cancer-associated microbiota is increasingly being recognized as one of the most significant components of the tumor microenvironment (Nejman et al., Science, (2020), 368:973, doi:10.1126/science.aay9189; Poore et al., Nature, (2020), 579:567-574, doi:10.1038/s41586-020-2095-1; Maman, S. & Witz, I. P., Nature Reviews Cancer, (2018), 18; 359-376). However, a complete map of the microbiota-host-drug network in cancer is lacking, mainly due to the difficulty in identifying the contribution of specific bacterial factors to both tumor development, progression and response to therapy. In this regard, several results show that the microbiota affects the host response to chemotherapeutic drugs (Lehouritis, et al., Sci Rep, (2015), 5:14554; Helmink et al., Nature Medicine, (2019), 25:377-388). Moreover, chemotherapeutics and antibiotics might further exacerbate any dysbiotic state rather than correct it, with potentially serious negative implications for drug response and toxicity (Alexander, et al., Nature Reviews Gastroenterology &Amp; Hepatology, (2017), 14:356, doi:10.1038/nrgastro.2017.20.). In particular, cancer-associated bacteria such as Mycoplasma hyorhinis¹⁰⁻¹² and Fusobacterium nucleatum¹³⁻¹⁶ can reduce the efficacy of certain anti-cancer drugs including gemcitabine, cisplatin and 5FU both in vivo and in vitro, though the molecular mechanism(s) involved are still largely unknown (Vande et al., J Biol Chem, (2014), 289:13054-13065; Liu et al., PLoS One, (2017), 12, e0184578; Geller et al., Science, (2017), 357:1156-1160; Zhang et al., Journal of Experimental & Clinical Cancer Research, (2019), 38: doi:10.1186/s13046-018-0985-y; Yamamura et al., Clinical Cancer Research, (2019), 25:6170-6179; Yu et al., Cell, (2017), 170: 548-563 e516; Gethings-Behncke et al., Cancer Epidemiology, Biomarkers & Prevention, (2020), 29: 539-548). Understanding how the many players involved in this extremely complex biological system interrelate would thus pave the way for the development of effective “precision medicine” strategies.

Colorectal and gastric cancers are among the leading cause of cancer-related mortality, both in the United States and worldwide (Sung et al., CA: A Cancer Journal for Clinicians, (2021), 71:209-249). Combination therapy comprising platinum-based molecules like cisplatin is widely used for the treatment of both cancers (Saber et al., BMC Cancer, (2018), 18:822, doi:10.1186/s12885-018-4727-5; Sui et al., Oncology letters, (2019), 17:944-950; Arai et al., BMC Cancer, (2019), 19:652, doi:10.1186/s12885-019-5720-3). Administration of either molecules results in DNA damage and RNA synthesis inhibition, leading to cell death through a series of cellular events not completely fully understood, but which also involve p53 activation (Kong et al., The Journal of biological chemistry, (2014), 289:27134-27145; Zamble et al., Proceedings of the National Academy of Sciences, (1998), 95:6163, doi:10.1073/pnas.95.11.6163; Bragado et al., Apoptosis: an international journal on programmed cell death, (2007), 12:1733-1742; Siddik et al., Oncogene, (2003), 22:7265-7279; Hagopian et al., Clinical Cancer Research, (1999), 5:655; Perrone et al., Journal of Clinical Oncology, (2010), 28:761-766; Hientz et al., Oncotarget, (2017), 8:8921-8946; Dasari et al., Eur J Pharmacol, (2014), 740:364-378; Longley et al., Nature Reviews Cancer, (2003), 3:330; Houghton et al., Clin Cancer Res, (1995), 1:723-730; Tchounwou et al., Journal of experimental pharmacology, (2021), 13:303-328).

We previously showed that Mycoplasma DnaK, a chaperone protein belonging to the Hsp70 family, binds to USP10 (ubiquitin carboxyl-terminal hydrolase 10), a regulator of p53 stability (Zella et al., Proceedings of the National Academy of Sciences, (2018), 115:E12005-E12014, doi:10.1073/pnas.1815660115). This binding in turn reduces the activities of p53, an essential transcription factor that promotes cell cycle blockage and apoptosis in the presence of extensive DNA damage (Zella et al., Proceedings of the National Academy of Sciences, (2018), 115:E12005-E12014, doi:10.1073/pnas.1815660115; Benedetti et al., Int J Mol Sci, (2020), 21, doi:10.3390/ijms21041311; Hafner et al., Nature Reviews Molecular Cell Biology, (2019), 20:199-210). Indeed, most anti-cancer drugs rely on the induction or blockage of DNA repair, with consequent activation of p53 followed by apoptosis to exert their function.

Here we investigated the effect of DnaK from Mycoplasma fermentans and Fusobacterium nucleatum on the anti-cancer activity of cisplatin. We showed that both DnaKs greatly reduces their efficacy, while a DnaK-binding peptidomimetic (Telaprevir) completely restored their anti-cancer activities. Current anti-cancer drugs regimens should take advantage of these data to design better personalized treatments.

### Mycoplasma and Fusobacterium DnaK Reduces the Activity of Anti-Cancer Drugs

To test the hypothesis that exogenously added Mycoplasma DnaK to cells treated with cisplatin could reduce their anti-cancer effect, we designed an in vitro assay based on the colorectal carcinoma cell line HCT116. This assay uses purified exogenous M. fermentans and F. nucleatus DnaK (eM-DnaK and eF-DnaK) and recapitulates the conditions whereby cancer cells would take up the bacterial protein released in the surrounding tumor microenvironment (Costa et al., et al., Nature Reviews Microbiology, (2015), 13:343, doi:10.1038/nrmicro3456; Carrio et al., J Bacteriol, (2005), 187: 3599-3601; Vega et al., The Journal of Immunology, (2008), 180:4299-4307). This in turn allowed us to study its effect on cell viability in the presence of the anti-cancer drugs.

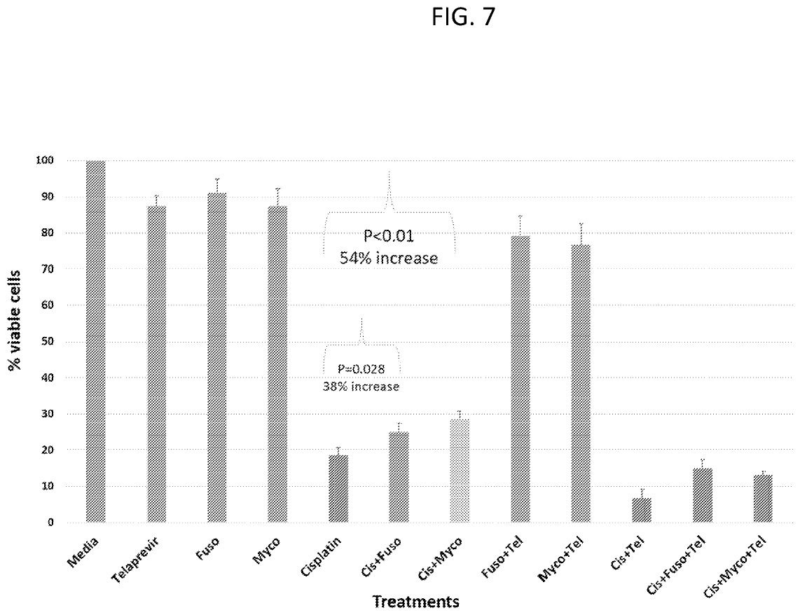

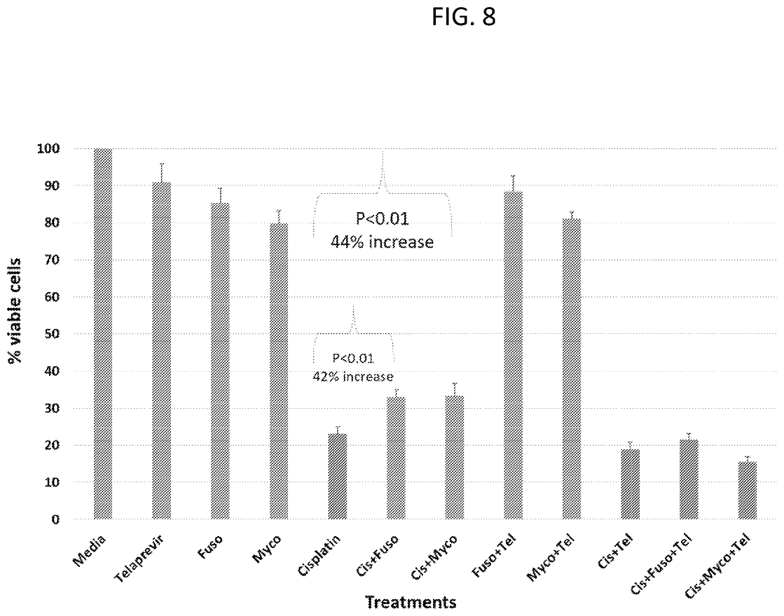

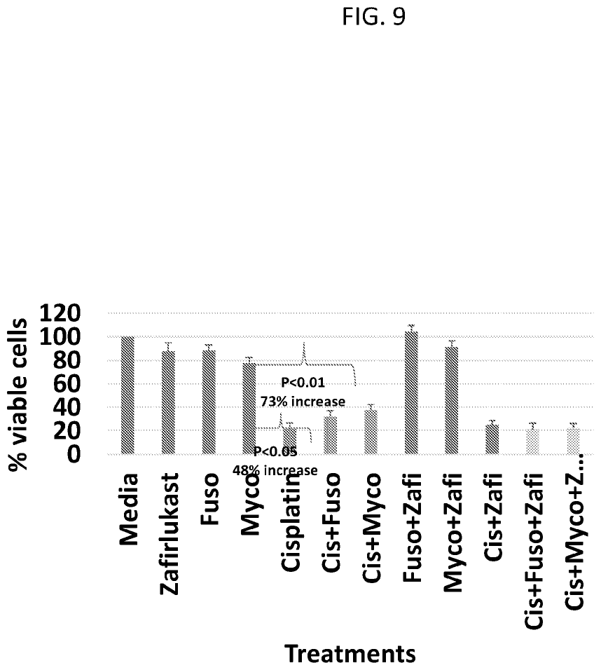

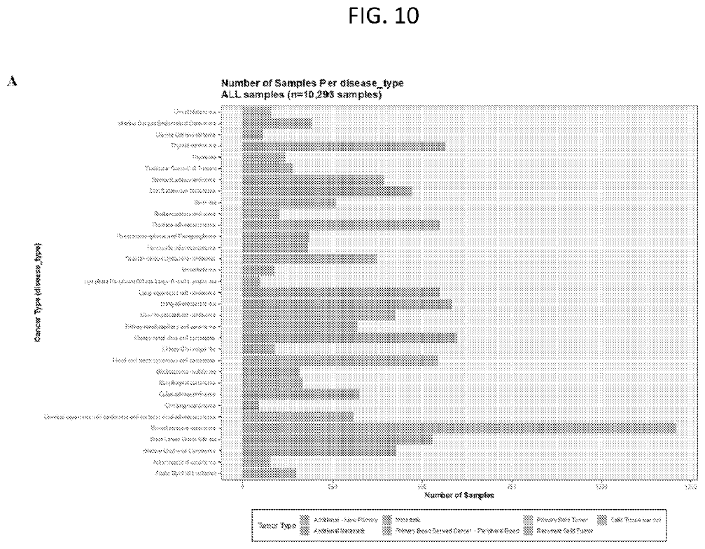

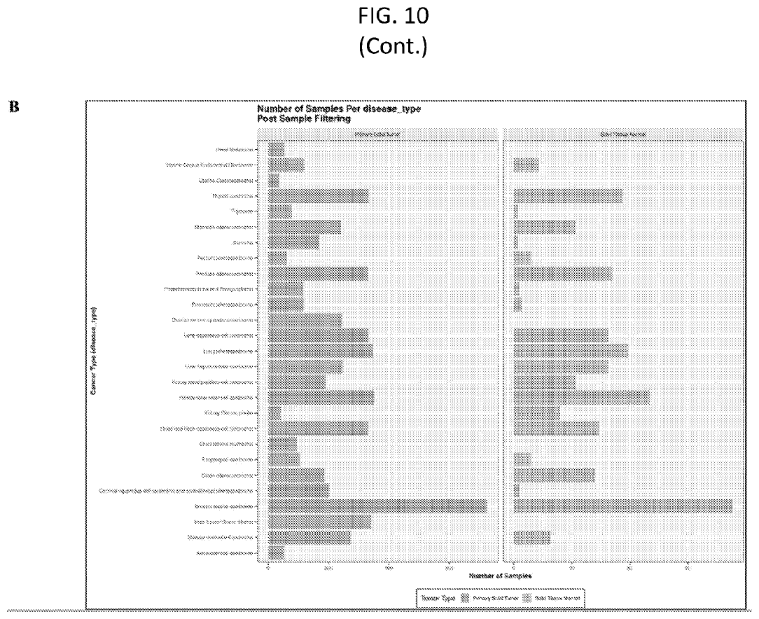

When HCT116 cells were treated with cisplatin in the presence of eM-DnaK or eF-DnaK, cell viability increased from below 20% to about 30%, indicating that the anti-cancer effect of the drug was blunted by about 40-50% (FIGS. 7 and 8).

### A Specific DnaK-Binding Compound, Telaprevir, Restores Cisplatin Anti-Cancer Activity

To provide proof of concept for therapeutic intervention, we used Telaprevir, a peptide-mimetic used to treat HCV, which has been previously demonstrated to bind DnaK and to reduce its ATPase activity (Hosfelt et al., Cell Chemical Biology, (2021), doi:https://doi.org/10.1016/j.chembiol.2021.11.004). In HCT116 cells treated with cisplatin in the presence of eM-DnaK or eF-DnaK and Telaprevir we observed a restoration of the original anti-cancer activity, indicating that the inhibitory effect of DnaKs was being reversed (FIG. 7).

Here we show that DnaK from either M. fermentans or F. nucleatum markedly hampers the anti-cancer effect of widely used anti-cancer drugs (cisplatin) in HCT116 colorectal cell lines. We also show that a DnaK binding peptidomimetic (Telaprevir) can fully reverse this inhibitory effect.

Based on our data, we propose that DnaK reaches the intracellular compartments by two routes: i) taken up by cancer cells after being expressed and secreted by bacteria present in the tumor microenvironment, and ii) by being expressed and secreted inside tumor cells by invading bacteria like Mycoplasmas or Fusobacteria (Benedetti et al., Int J Mol Sci, (2020), 21, doi:10.3390/ijms21041311; Theriault et al., J Immunol, (2006), 177:8604-8611; Zella et al., Proceedings of the National Academy of Sciences, (2018), 115:E12005, doi:10.1073/pnas.1815660115; Curreli et al., International Journal of Molecular Sciences, (2021), 22, 3885; Taylor-Robinson et al., Int J Exp Pathol, (1991), 72:705-714; Brennan et al., Nature reviews. Microbiology, (2019), 17:156-166). Intracellular DnaK then binds and reduces the activity of host proteins (such as p53) involved in the response to certain anti-cancer drugs (Zella et al., Proceedings of the National Academy of Sciences, (2018), 115:E12005-E12014, doi:10.1073/pnas.1815660115; Benedetti et al., Int J Mol Sci, (2020), 21, doi:10.3390/ijms21041311; Curreli et al., International Journal of Molecular Sciences, (2021), 22, 3885). DnaK interaction with co-chaperon proteins, including the co-chaperone DnaJ, could provide the necessary ATPase activity for efficiently “sample” client substrates and function as a chaperone inside the eukaryotic cell. Since Telaprevir binds the DnaK ATP-ase region and inhibits the ATP-ase activity, the peptide likely then acts by “locking” DnaK in a conformation unable to bind and inhibit the client proteins' functions, thus restoring the drugs' anti-cancer activity (Zella et al., Proceedings of the National Academy of Sciences, (2018), 115:E12005-E12014, doi:10.1073/pnas.1815660115; Clerico et al., J Mol Biol, (2015), 427:1575-1588; Kragol et al., Biochemistry, (2001), 40:3016-3026; Ostorhazi et al., Biopolymers, (2011), 96:126-129).

We note that these bacteria were previously shown to reduce the anti-cancer activity of drugs like cisplatin both in vitro and in vivo, though the molecular mechanisms responsible for this effect are still largely unknown (Liu et al., PLoS One, (2017), 12, e0184578; Zhang et al., Journal of Experimental & Clinical Cancer Research, (2019), 38: doi:10.1186/s13046-018-0985-y; Yamamura et al., Clinical Cancer Research, (2019), 25:6170-6179; Yu et al., Cell, (2017), 170: 548-563 e516; Gethings-Behncke et al., Cancer Epidemiology, Biomarkers & Prevention, (2020), 29: 539-548). In addition, it has been shown that intra-tumoral F. nucleatum levels predict therapeutic response to neoadjuvant chemotherapy in esophageal squamous cell carcinoma. Consequently, our results not only highlight a novel mechanism used by cancer-associated bacteria to counteract the effect of anti-cancer drugs, but also establish a prospective tool for therapeutic intervention. This would provide a practical framework for the design and implementation of novel, personalized diagnostic and therapeutic anti-cancer strategies by targeting specific bacterial DnaKs in patients with poor response to chemotherapy.

### Materials and Methods

### Cell Lines

Human colorectal carcinoma cell line (HCT116) used in the experiments were all from American Type Culture Collection (ATCC). The cells were cultured in a humidified incubator at 37° C. in 5% CO₂ in McCoy medium, containing 10% fetal bovine serum (FBS), 100 U/ml penicillin, 100 U/ml streptomycin and 290 μg/mL L-glutamine (all from ThermoFisher Scientific, Waltham, MA, USA).

Expression and Purification of Mycoplasma fermentans (eM-DnaK) and Fusobacterium nucleatum (eF-DnaKs) Exogenous Proteins

Recombinant exogenous DnaKs-V5 were obtained as previously described (Zella et al., Proceedings of the National Academy of Sciences, (2018), 115:E12005-E12014, doi:10.1073/pnas.1815660115; Benedetti et al., Journal of Translational Medicine, (2021), 19:60, doi:10.1186/s12967-021-02734-4). Briefly, both eMF-DnaK and eF-DnaK sequences were inserted into a cloning vector for the expression of the protein. They were then subcultured into TB/LB with Kanamycin, followed by fractionation and purification (Biomatik USA, Wilmington, DE). After this step, the proteins were extensively dialyzed against PBS 1× (pH 7.4), and Coomassie blue-stained SDS-PAGE (>85%) was used to determine their purity. The proteins were then aliquoted to avoid frequent freeze-thaws and kept at −80° C. after reconstitution.

Treatments with Anti-Cancer Drugs (Cisplatin) and Telaprevir

To determine the effects of eM and eF-DnaKs on HCT116 cell line treated Cisplatin, cells were plated 300,000 cells/well in 6-wells plates. After 24 h, both eM-DnaK and eF-DnaK were added to the cultures at a concentration of 10 ug/ml. After 24 h, Cisplatin (25 μM) was added to the cells (both treated and not treated with DnaKs). Cisplatin is from Selleckchem (Houston, TX). Parallel cultures of untreated cells were the negative control. Also, parallel treatments of DMF (control for Cisplatin treatment, dissolved in DMF following manufacturer's instructions) have been used as negative controls. Cells treated with DMF did not show increased cell death and their proliferation rate remain normal. After 48 h of treatment with the anti-cancer drugs, cell monolayers were washed in PBS, trypsinized and cell viability was measured using the trypan blue assay. The trypan blue exclusion assay allows for a direct identification and enumeration of live (unstained) and dead (blue) cells in the given population.

To confirm that bacterial DnaK was responsible for reduction in platinum-based drugs, we used a peptide (Telaprevir) which has been previously demonstrated to bind to M. tuberculosis DnaK substrate-binding domain and to reduce its ATPase activity. More in detail, we pre-treated both DnaKs with Telaprevir (25 μg/ml) before adding them to the culture of HCT116 cells. After 3 h of incubation the complex was added to the culture. After 24 h the cells were treated with the anti-cancer drugs and then subjected to count with trypan blue as previously described. Parallel cultures of cells treated with the drugs and DnaKs not complexed with Telaprevir were used as control.

### Example 5. Bacterial DnaK Reduces the Activity of Anti-Cancer Drugs Cisplatin and 5FU

To investigate the role of DnaK in chemotherapy, we treated cancer cell lines with M. fermentans DnaK and then with commonly used p53-dependent anti-cancer drugs (cisplatin and 5FU). We evaluated the cells' survival in the presence or absence of a DnaK-binding peptide (ARV-1502). We also validated our findings using primary tumor cells from a novel DnaK knock-in mouse model. To provide a broader context for the clinical significance of these findings, we investigated human primary cancer sequencing datasets from The Cancer Genome Atlas (TCGA). We identified F. nucleatum as a CAB carrying DnaK with an amino acid composition highly similar to M. fermentans DnaK. Therefore, we investigated the effect of F. nucleatum DnaK on the anti-cancer activity of cisplatin and 5FU.

Our results show that both M. fermentans and F. nucleatum DnaKs reduce the effectiveness of cisplatin and 5FU. However, the use of ARV-1502 effectively restored the drugs' anti-cancer efficacy. Our findings offer a practical framework for designing and implementing novel personalized anti-cancer strategies by targeting specific bacterial DnaKs in patients with poor response to chemotherapy, underscoring the potential for microbiome-based personalized cancer therapies.

Here we investigated the effect of M. fermentans DnaK on the anti-cancer activity of cisplatin and 5FU. We showed that DnaK exogenously added to human cancer cell lines greatly reduces the efficacy of both anti-cancer drugs, while a DnaK-binding peptide completely restored their activity. Next, we confirmed these data in primary tumor cells from a knock-in mouse model constitutively expressing DnaK generated in our laboratory. By mining human primary cancer sequencing datasets from The Cancer Genome Atlas (TCGA) we then detected other CAB carrying DnaKs with highly similar amino acid composition. Among them, we identified F. nucleatum and demonstrated that also its DnaK can inhibit the anti-cancer efficacy of cisplatin and 5FU when exogenously added to cancer cell lines. In conclusion, we highlight a new mechanism whereby bacteria hamper anti-cancer effects of widely used chemotherapeutic agents. Current anti-cancer drugs regimens should consider these data to design better personalized treatments in cancer patients when planning treatment protocols or when considering causes of failing regimens.

### Methods

### Cell Lines

A human colorectal carcinoma cell line (HCT116) and a gastric adenocarcinoma cell line (AGS) used in the experiments were all from American Type Culture Collection (ATCC). The cells were cultured in a humidified incubator at 37° C. in 5% CO₂ in McCoy medium (HCT116) or F-12K medium (Kaighn's Modification of Ham's F-12 medium) (AGS), all containing 10% fetal bovine serum (FBS), 100 U/ml penicillin, 100 U/ml streptomycin and 290 μg/mL L-glutamine (all from ThermoFisher Scientific, Waltham, MA, USA).

Expression and Purification of Mycoplasma fermentans (eM-DnaK) and Fusobacterium nucleatum (eF-DnaKs) Exogenous Proteins

Recombinant exogenous DnaKs-V5 used in this study were obtained as previously described (Zella et al., Proceedings of the National Academy of Sciences, (2018), 115:E12005-E14; Benedetti et al., Journal of Translational Medicine, (2021), 19:60). Briefly, both eM-DnaK and eF-DnaK sequences were inserted into a cloning vector for the expression of the protein fused to a V5-tag peptide. They were then subcultured into TB/LB with Kanamycin, followed by fractionation and purification (Biomatik USA, Wilmington, DE). After this step, the proteins were extensively dialyzed against PBS 1× (pH 7.4), and Coomassie blue-stained SDS-PAGE (>85%) was used to determine their purity. The proteins were then aliquoted to avoid frequent freeze-thaws and kept at −80° C. after reconstitution.

### Dnak Knock-In Mice and Isolation of Primary Cells

Transgenic animals were generated in collaboration with Taconic Biosciences (Rensselaer, NY). Briefly, the “CAG-Kozak-DnaK-V5 tag-TAGTAG-polyA” cassette was cloned into intron 1 of ROSA26 in reverse orientation. The V5 tag was added to conveniently detect DnaK, which was inserted in the ROSA26 locus by using the CRISPR/Cas9-mediated genome editing technology. The University of Maryland School of Medicine Institutional Animal Care and Use Committee approved these experiments. Mice were monitored daily and when a spontaneous solid tumor mass was detected, the mouse was euthanized, and the mass carefully removed. A portion of the tumor mass was placed in formalin and then sent to the American Histolabs (Gaithersburg, MD) for the paraffin embedding and the Hematoxylin and Eosin staining of the slides. Pictures of the slides has been taken using an Olympus BX43 microscope (DP72 camera) and the CellSens Standard software (Olympus). The rest of the cancer cells were separated in a single-cell suspension from the intact tissue by mechanical force and then cultured under normal culturing conditions in RPMI+10% FBS (37° C., 5% CO₂) and partially frozen at −80° C.

Treatments with Anti-Cancer Drugs (Cisplatin and 5FU) and ARV-1502 Peptide

To determine the effects of eM and eF-DnaKs on HCT116 and AGS cells lines treated with different anti-cancer drugs, cells were plated 200,000 cells/well in 6-wells plates. After 24 h, both eM-DnaK and eF-DnaK were added to the cultures at a concentration of 10 ug/ml. After 24 h, anti-cancer drugs (cisplatin 2 μM, 5FU 75 μM) were added to the cells (both treated and not treated with DnaKs). We selected these concentrations of platinum-based drugs or 5FU to decrease the number of viable cells by at least 50%. Cisplatin is from Selleckchem (Houston, TX), while 5FU is from Sigma-Aldrich (St. Louis, MO). Parallel cultures of untreated cells were the negative controls. Also, parallel treatments of DMF (control for cisplatin treatment, dissolved in DMF following manufacturer's instructions) and DMSO (control for 5FU treatment, dissolved in DMSO based on manufacturer's instructions) have been used as negative controls. Cells treated with DMF or DMSO did not show increased cell death and their proliferation rate remain normal. Thus, we used untreated cells as negative control. After 48 h of treatment with the anti-cancer drugs, cell monolayers were washed in PBS, trypsinized and cell viability was measured using the trypan blue assay. The trypan blue exclusion assay allows for a direct identification and enumeration of live (unstained) and dead (blue) cells in the given population.

To verify that bacterial DnaK was responsible for reduction in platinum-based drugs and 5FU anti-cancer-activities, we used a peptide (ARV-1502, optimized from pyrrhocoricin and drosocin) which binds to Escherichia coli DnaK substrate-binding domain and to decreases its ATPase activity (Kragol et al., Biochemistry, (2001), 40:3016-26; Ostorhazi et al., Biopolymers, (2011), 96:126-9; Otvos et al., International Journal of Peptide Research and Therapeutics, (2005), 11:29-42; Otvos et al., Biochemistry, (2000), 39:14150-9). More in detail, we pre-treated both exogenous DnaKs with ARV-1502 (25 μg/ml) before adding them to the culture of HCT116 or AGS cells. After 3 h of incubation the complex was added to the culture. After 24 h the cells were treated with the anti-cancer drugs and then subjected to count with trypan blue as previously described. Parallel cultures of cells treated with the drugs and DnaKs not complexed with ARV-1502 were used as control.

We followed the same experimental procedures described above for the treatment of the primary murine cancer cells (ex vivo experiments). In particular, primary cancer cells from DnaK knock-in mice were treated with the same concentrations of anti-cancer drugs (cisplatin and 5FU) and with the same concentration of DnaK inhibitor (ARV-1502), for the duration of the experiments. As before with the exogenous DnaKs, the primary cancer cells have been pretreated with ARV-1502 for 24 h before adding the anti-cancer drugs.

Statistical differences in the means were tested using Student's t test. All statistical tests were two-sided.

### Western Blotting

Western blot was performed to verify the expression of DnaK in the mouse primary cancer cells, in the internalization of the exogenous DnaKs and to validate DnaK binding with ARV-1502. HCT116 cells were treated with eM-DnaK, with or without ARV-1502, with or without Cisplatin, as described in the previous section of Methods. eM-DnaK was added to the cells at a concentration of 10 ug/ml. After 72 h since eM-DnaK treatment, cell monolayers were washed in cold PBS, trypsinized and resuspended in RIPA lysis buffer (Sigma-Aldrich, St. Louis, MO) in the presence of protease inhibitors (Sigma-Aldrich, St. Louis, MO). The protein concentration was measured by the Bradford assay (Bio-Rad Laboratories, Hercules, CA). Thirty micrograms of protein were resolved by SDS/PAGE, transferred to a polyvinylidene difluoride (PVDF) membrane using trans-blot turbo transfer system (Bio-Rad Laboratories, Hercules, CA), blocked in 5% nonfat dried milk in Tris-Buffered Saline (TBS) and probed overnight with either a mouse mAb against the V5 tag (#R960-25, Thermo Fisher Scientific, Walthman, MA) to detect the presence of eM-DnaK or a mouse mAb against 3-actin (8H10D10) (#3700, Cell Signaling Technology, Danvers, MA). Blots were then incubated with a secondary HRP-conjugated antibody (Cell Signaling Technology, Danvers, MA) and developed using an ECL chemiluminescent substrate kit (Genesee Scientific, San Diego, CA). They were then exposed and acquired using the ChemiDoc MP digital image system (Bio-Rad Laboratories, Hercules, CA).

The untreated primary cancer cells underwent the same procedures. Briefly, the total proteins were extracted and quantified, and after running and blotting, the membranes were probed overnight with either a primary rabbit mAb antibody against the V5 tag (#ab182008, Abeam) to detect the presence of DnaK-V5, or a rabbit mAb against GAPDH (14c10) (#2118S, Cell Signaling Technology, Danvers, MA) used as housekeeping. We resolved in the same gel eM-DnaK protein as positive control and proteins obtained from a spleen of a DnaK^{-/-} animal as negative control.

### Surface Plasmon Resonance (SPR) Binding Analysis of DnaK-ARV-1502

Surface plasmon resonance (SPR) binding studies of DnaK and ARV-1502 were performed at 25° C. on a BIAcore T100 System (BIAcore, Inc., Piscataway, NY). We used as assay buffer HBS-EP, containing 10 mM HEPES, 150 mM NaCl, 0.05% surfactant P20, pH 7.4, 3 mM EDTA. DnaK (2274.9 RUs) was immobilized on CM5 sensor chips using the amine-coupling chemistry recommended by the manufacturer. Analytes were introduced into the flow cells at 35 μl/min in the running buffer. Association and dissociation were assessed for 250 seconds and 600 seconds. Resonance signals were corrected for nonspecific binding by subtracting the background of the control flow-cell. After each analysis, the sensor chip surfaces were regenerated with 10 mM glycine solution (pH 2.0) with MgCl 1M and equilibrated with the buffer before the next injection.

### TCGA Analysis

The presence and distribution of bacteria within human cancer tissues, especially Mycoplasma and Fusobacterium, was assessed through data mining of human primary cancer sequencing datasets from The Cancer Genome Atlas (TCGA). TCGA hosts human genomic and transcriptomic sequencing data sets from a large number of human cancer tissues, where bacterial sequences can also be retrieved and analyzed to characterize CAB (The Cancer Genome Atlas Research et al., Nature Genetics, (2013), 45:1113).

RNA-Seq sequences from a total of 10,293 samples spanning 33 different cancer types were initially retrieved from TCGA (version 9.0), after which analyses were focused only on primary tumor samples and solid tissue normal samples. As such, samples from the following cancers were removed from the analyses: acute myeloid leukemia, lymphoid neoplasm diffuse large B-cell lymphoma, mesothelioma, skin cutaneous melanoma, cholangiocarcinoma, testicular germ cell tumors, as well as metastatic, additional metastatic and “additional—new primary” samples. After sample filtering, the final dataset analyzed was comprised of 9,505 primary solid tumor and solid tissue normal samples distributed across 27 cancer types. To note, some solid tissue normal samples (uveal melanoma, uterine carcinosarcoma, ovarian serous cystadenocarcinoma, glioblastoma multiforme, brain lower grade glioma, adrenocortical carcinoma) were missing in the dataset. Sequences were downloaded in BAM alignment format from TCGA, and reads which were indicated in the alignment as mapping to the human genome were discarded, since we needed to retain only the potential microbial sequences. To distinguish microbial sequences from other sequences that did not map to the human genome (such as sequencing artifacts or mutations/rearrangements within tumors) we first screened the sequences with a Hidden Markov Model (HMM) created from the SILVA Release 132 alignment and then taxonomically classified the sequences using Kraken 2 with a database also based on SILVA 132 (Yoon et al., Current genomics, (2009), 10:402-15; Quast et al., Nucleic acids research, (2013), 41:D590-D6; Wood et al., Genome Biology, (2019), 20:257). The resulting 16S sequence dataset was taxonomically assigned to a total of 9,510 taxa at 7 different taxonomic levels (from phylum to species) and count tables were generated for data visualization and analyses in R. Because of the wide variations in the number of reads sequenced across all samples (min: 49,637,151 sequencing reads; max: 516,415,337 sequencing reads; FIG. 19 top panel), 16S counts were then normalized in each sample by computing a scaling factor based on the number of reads in a sample divided by the number of reads in the smallest sample.

Specimen Collection and Detection of Bacterial DnaK Using qPCR

Frozen biopsies of cancers tissues already identified in the Pathology Biorepository Shared Service (PBSS) core of the Greenebaum Comprehensive Cancer Center at the University of Maryland (GCCC-UM) were collected and stored in deep freezer (−80° C.). This retrospective study was approved by the Institutional Review Board at University of Maryland, Baltimore (approval number: HP-00040021). All methods were performed following the relevant guidelines and regulations. Documented informed consent was obtained from each study participant. Patient demographics and clinical characteristics were investigated by reviewing the medical records and interviews. Minimal associated clinic-pathologic data to include tumor histologic type, treatment status (treatment naïve vs. post neoadjuvant treatment), treatment regimen, and an assessment of patient treatment responses was collated.

Total DNA was extracted from tissues using the DNeasy Blood & Tissue Kits (QIAGEN, Hilden, Germany) according to the manufacturer's instructions. DNA concentration and purity were recorded using a NanoDrop spectrophotometer (NanoDrop Technologies, Wilmington, DE). Mycoplasma and Fusobacterium DnaK genes were detected and amplified by qPCR using the following primers and probes:

-eM-DnaK:
F primer:
CAA TGC ACA ACG TGA AGC CA;
R primer:
AAG CAG CAG CAG TAG GTT CG;
probe:
5 6-FAM/AT CGC AGG T/ZEN/A AAA TTG CAG G/
3IABkFQ/;
-eF-DnaK:
F primer:
CAA CAC AAG GAC CTA CAA AAA C;
R primer:
CGC AAC AAC TTC ATC AGG G;
probe:
/56-FAM/AA ATC TTA C/ZEN/T TGT TGG AGG
TTC TAC AAG AAT ACC A/3IABKFQ/.

Briefly, amplifications were performed in 20p1 reaction mixture containing 1×SsoAdvanced Universal Probes Supermix (Bio-Rad Laboratories, Hercules, CA), each primer at 300 nM, probe at 200 nM and 50 ng of total DNA. Reference standard curves were generated using serially diluted plasmids containing the target DnaK gene. Aliquots were prepared once by dilution of DNA in distilled water and were stored at −20° C. Water and aliquots of total DNA from HCT116 and AGS cells Mycoplasma and Fusobacterium-free were included for each of the amplifications as negative controls.

Following activation of DNA polymerase at 94° C. for 30 s, 40 cycles of amplification (denaturation step, 95° C. for 15 s; annealing-extension step, 60° C. for 30 s) were performed with CFX384 Real-Time PCR System (Bio-Rad Laboratories, Hercules, CA). An accurate analysis of the melting temperature curve of the generated amplicons was conducted for of the amplifications to rule out any non-specific interference.

### Generation of Data for DnaK Domains' Comparison

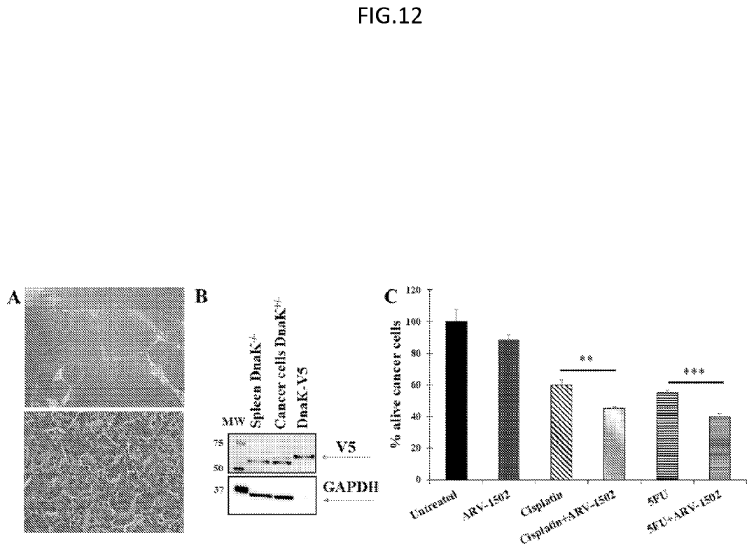

We started from the Mycoplasma fermentans (MF-I1—ATFG00000000)(30) template sequence for DnaK. The template sequence is reported in Table 3. We extracted three domains for DnaK. The exact positions of the regions of interested are reported, separated by semicolons, in the respective sequence headers. Domain 1 (NDB) extended within aa1-392, domain 2 (SBD) within aa392-507, and finally domain 3 (α-helical domain) within aa508-638, as described (49). We downloaded 22,155 DnaK bacterial proteins from NCBI (query: DnaK_hsp70). We then aligned each fragment of each template against ncbi-blast-2.9.0 against the target downloaded proteins. Blast results were filtered to keep only matches >70% of the length of the query, after which the genus information of the matches was parsed to generate the distribution plots in FIG. 12C.

TABLE 2
Template sequence used for DnaK domains' comparison (SEQ ID NO: 23)
>Mycoplasma fermentans no info; 1-366; 367-481; 482-585
MAKETIIGIDLGTTNSAVAIVDGGTPIVLENYNGKRTTPSVVSFKDGEIIVGENAKNQIETNPDTIAS
VKRFMGTKKIFKANGKEYKPEEISAIILDHLRKYAEEKVGHKIEKAVITVPAYFDNAQREATKIAGKI
AGLDVLRIINEPTAAALAFGLDKTNKEMKVLVFDLGGGTFDVSILELADGTFEVLATSGDNKLGGDDW
DHEIVDWLVAKIKNDHKIDIRENKMAMARLKAAAEKAKIDLSSSLVAHISLPFLVLLDNHEPINVEAE
LKRSEFEKMTAKLVERCRRPIQDALSEAKLKISDLDEILLVGGSTRIPAVQALVEKILNRKPNKSVNP
DEVVAMGAAIQGAVLAGDINDILLVDVTPLILGIETAGGISTPLIPRNTRIPITKSETFTTFENNQTD
VTIKIVQGERPVASENKLLGQFNLTGIRPAPRGIPQIEVSFKIDANGITTVSAKDKDTQKEQSITIKN
SSKLSEEEVERMIKEAEENREADAKRAADIEIIVRAETMVAKFESVLEENKDKLTQDQINQAQAEIDK
INGFIKEKEYDQLRLTIKAFEELLDSMSNADSSSFKEEDAE

### Results

### Exogenous Mycoplasma DnaK Reduces the Activity of Cisplatin and 5 Fluorouracil in Human Cancer Cell Lines

To test the hypothesis that exogenously Mycoplasma DnaK added to cells treated with cisplatin or 5FU could reduce their anti-cancer effect, we designed an in vitro assay with the colorectal carcinoma cell line HCT116 and the gastric carcinoma cell line AGS. This assay, which uses purified exogenous M. fermentans DnaK (eM-DnaK), would recapitulate the conditions whereby cancer cells would take up the bacterial protein released in the surrounding tumor microenvironment (Zella et al., Proceedings of the National Academy of Sciences, (2018), 115:E12005-E14; Carrio et al., J Bacteriol, (2005), 187:3599-601; Theriault et al., J Immunol, (2006), 177:8604-11; Costa et al., Nature Reviews Microbiology, (2015), 13:343; Vega et al., The Journal of Immunology, (2008), 180:4299-307). This in turn allowed us to study DnaK's effect on cell viability in the presence of the anti-cancer drugs.

When HCT116 cells were treated with cisplatin in the presence of eM-DnaK, the anti-cancer effect of the drug was blunted, and viability greatly increased from 31% to 53% (FIG. 12A). On the other hand, the presence of eM-DnaK did not have a statistically significant effect on the anti-cancer action of 5FU, and viability increased only from 14% to 16% (FIG. 12B).

When AGS cells were treated with cisplatin in the presence of eM-DnaK the anti-cancer effect of the drug was also reduced, and cell viability increased from 40% to 57% (FIG. 12C). Similarly, a reduction of anti-cancer activity was also observed following treatment with 5FU in the presence of eM-DnaK, with viability increasing from 15% to 33% (FIG. 12D).

### A Specific DnaK-Binding Peptide Restores the Drugs' Anti-Cancer Activities

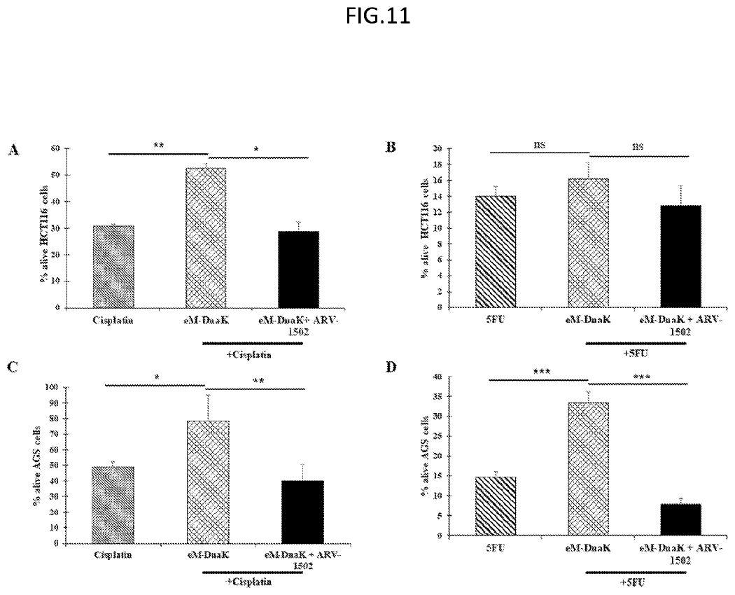

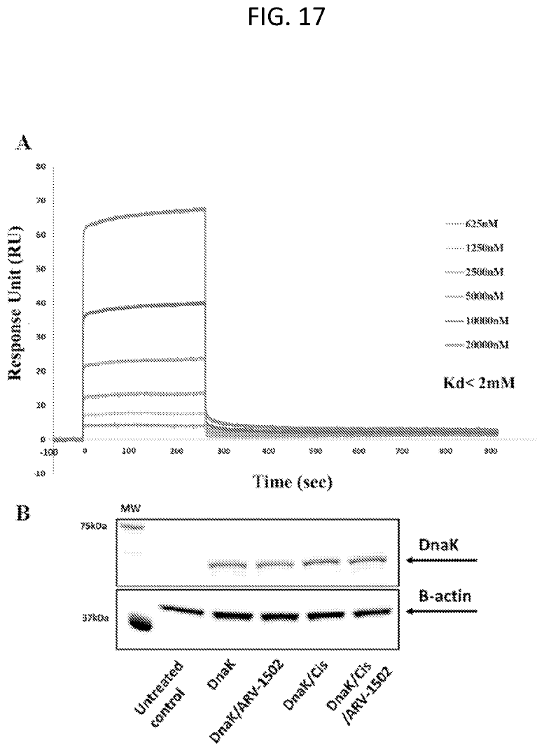

To confirm that bacterial DnaK is responsible for reduction in cisplatin and 5FU anti-cancer-activities, and to provide proof of concept for therapeutic intervention, we used ARV-1502, a peptide optimized from pyrrhocoricin and drosocin, which has been previously demonstrated to bind the Escherichia coli DnaK substrate-binding domain and to reduce its ATPase activity, without interacting with human Hsp70 (Kragol et al., Biochemistry, (2001), 40:3016-26; Otvos et al., Frontiers in chemistry, (2018), 6:309; Otvos et al., Journal of Medicinal Chemistry, (2005), 48:5349-59). We first show that the peptide is also able to bind eM-DnaK (FIG. 17A) and that ARV-1502 binding to DnaK is not preventing the exogenous protein entry into the cells (FIG. 17B). Then, we proceeded to analyze the effects of ARV-1502 in HCT116 and AGS cells treated with cisplatin and 5FU in the presence of eM-DnaK. In all samples we observed a restoration of the original anti-cancer activity of each drug, indicating that the inhibitory effect of DnaKs was being reversed (FIG. 11A-D).

### Arv-1502 Increases the Activity of Anti-Cancer Drugs in Mouse Primary Cancer Cells Expressing Mycoplasma DnaK Protein.

To validate our previous data in cell lines, we next used primary cancer cells derived from a spontaneous solid tumor mass (round cell neoplasia) retrieved from the abdomen of a DnaK positive knock-in mice generated in our Laboratory (FIG. 12A). These cells constitutively express DnaK mimicking an in vivo situation whereby the cells would be infected by M. fermentans expressing and secreting DnaK inside the cell's compartments (FIG. 12B) (Curreli et al., International Journal of Molecular Sciences, (2021), 22:3885). Treatment of the cancer cells with cisplatin or 5FU reduced their viability to 60% and 55%, respectively (FIG. 12C). The treatment with the DnaK inhibitor ARV-1502 alone had a slight inhibitory effect (11%), resulting in reduced cells viability to 89% compared to the untreated cells. When the primary cancer cells were treated with the DnaK inhibitor ARV-1502 we observed cell viability further reduced to 45% for cisplatin (FIG. 12C, left panel) and to 40% for the 5FU (FIG. 12C, right panel), which amounts to a 25% improved anti-cancer effect for both drugs. These data indicate that inhibiting DnaK activity re-established the activity of anti-cancer drugs in the primary cancer cells, confirming the data obtained in the cancer cell lines.

Identification of CAB with Amino Acid Composition Similar to Mycoplasma DnaK

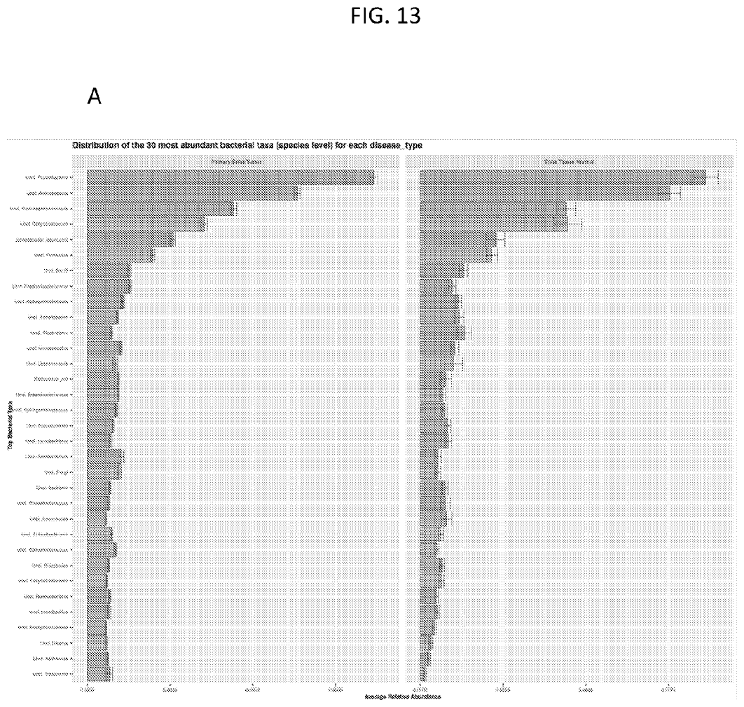

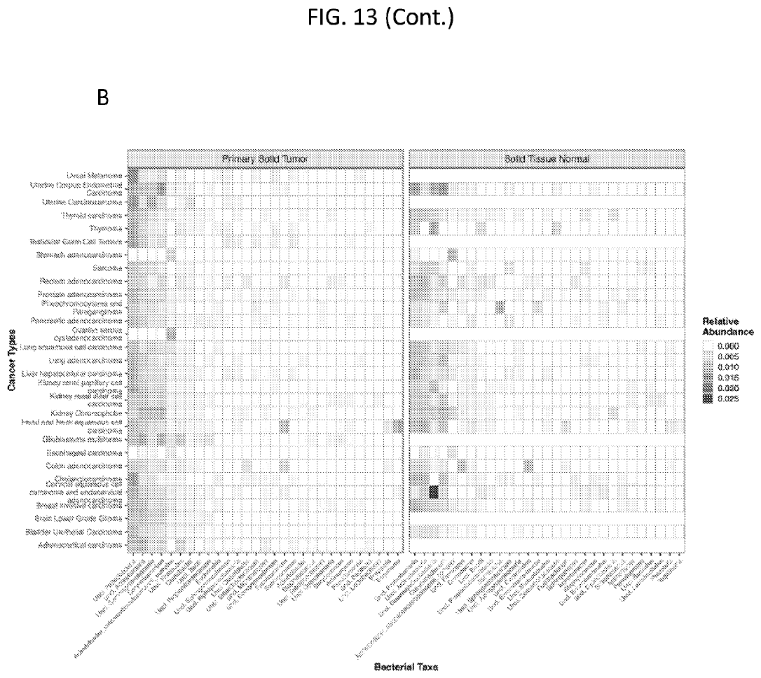

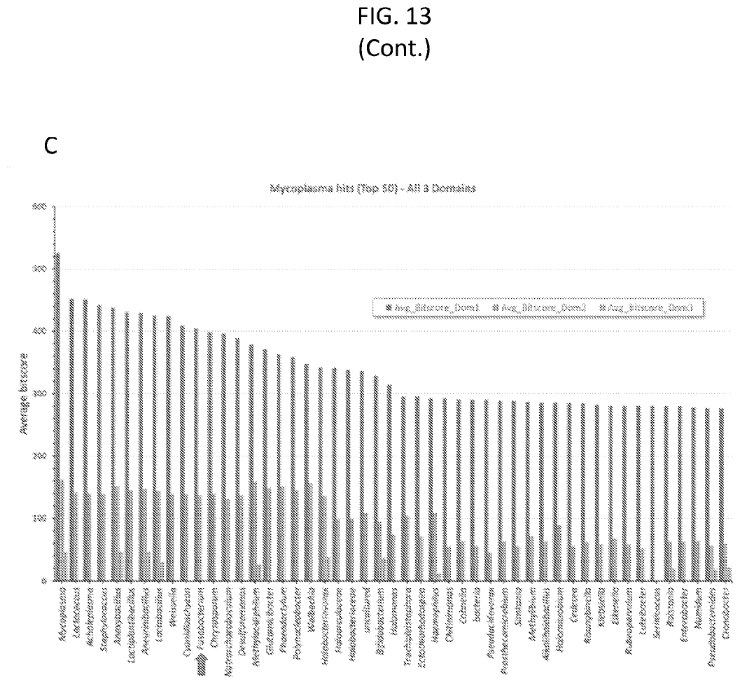

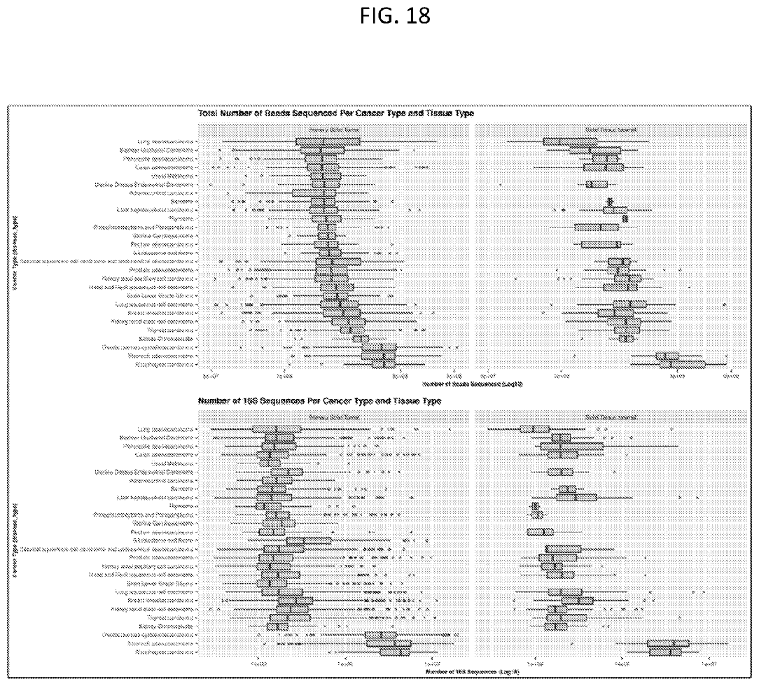

The Cancer Genome Atlas provides a comprehensive dataset of nucleic acid sequences, both DNA and mRNA from a number of cancer tissues (Poore et al., Nature, (2020), 579:567-74; The Cancer Genome Atlas Research et al., Nature Genetics, (2013), 45:1113; Dohlman et al., Cell Host & Microbe, (2021), 29:281-98.e5). We reasoned that bacterial sequences could be retrieved from this dataset and used to evaluate the composition of the cancer-associated microbiota and the expression of different bacterial genes, after removal of all the eukaryotic sequences from the mRNA dataset (see also Materials and Methods). Using the 16S rRNA gene sequences identified in the TCGA data set, we characterized bacterial taxa profiles from 9,505 primary solid tumor and solid tissue normal samples distributed across 27 cancer types (FIG. 18). On average, 263,379 16S rRNA reads were identified in each cancer type with a wide variability across cancer types (from a min of 79 16S sequences in kidney renal carcinomas to 38,415,049 16S sequences in ovarian serous cystadenocarcinoma; FIG. 18 bottom panel) which was concomitant with the wide range in the sequencing data set size for each sample. Overall, the top 5 bacterial taxa detected across all samples (both primary solid tumor and solid tissue normal) were Proteobacteria, Actinobacteria, Gammaproteobacteria, Corynebacterium (all four taxa unclassified at the genus level) and Acinetobacter baumanii bacteria (FIG. 13A). The general bacterial profiles, aggregating samples across all cancers, seemed similar when comparing primary solid tumor to solid tissue normal samples (FIG. 13B). Nonetheless, previous studies have shown that bacterial biomarkers of cancer can be identified for specific cancer types and that specific bacteria compose the tumor microbiome, hinting at potential role of specific bacteria in certain cancers (Poore et al., Nature, (2020), 579:567-74; Adlung et al., Nature Cancer, (2020), 1:379-81; Zackular et al., Cancer Prevention Research, (2014), 7:111; Nejman et al., Science, (2020), 368:973-80; Riquelme et al., Cell, (2019), 178:795-806.e12).

To identify other DnaKs that could have the same inhibitory effect on anticancer drugs, we searched for bacterial DnaKs with amino acid composition similar to the different domains of Mycoplasma DnaK, which consists of an N-terminal ATPase domain of about 45 kDa (NBD, nucleotide binding domain) and a C-terminal substrate of about 25 kDa (SDB, substrate binding domain). The latter is further subdivided into a β-sandwich subdomain of about 15 kDa and a C-terminal α-helical subdomain of 10 kDa (Mayer et al., Cellular and Molecular Life Sciences, (2005), 62:670). We obtained a list of the first 50 bacterial hits by average bitscore for each domain of Mycoplasma DnaK, which is presented in FIG. 13C. Among the identified bacteria, Fusobacterium DnaK exhibited a high degree of similarity with M. fermentans DnaK. These results extend and confirm at the domain level our previous phylogenetic amino acid analysis (Zella et al., Proceedings of the National Academy of Sciences, (2018), 115:E12005-E14). We note that several reports indicate that F. nucleatum is commonly associated with gastrointestinal cancer and progression with cancer and resistance to anti-cancer therapy (Kostic et al., Cell Host Microbe. 2013; 14(2):207-15; Fukugaiti et al., Braz J Microbiol. 2015; 46(4):1135-40; Mima et al., Gut, (2016), 65:1973-80; Zhang et al., Journal of Experimental & Clinical Cancer Research, (2019), 38:14; Yamamura et al., Clinical Cancer Research, (2019), 25:6170-9; Yu et al., Cell, (2017), 170:548-63 e16).

The Activity of Anti-Cancer Drugs is Reduced by Fusobacterium nucleatum DnaK and Restored by ARV-1502

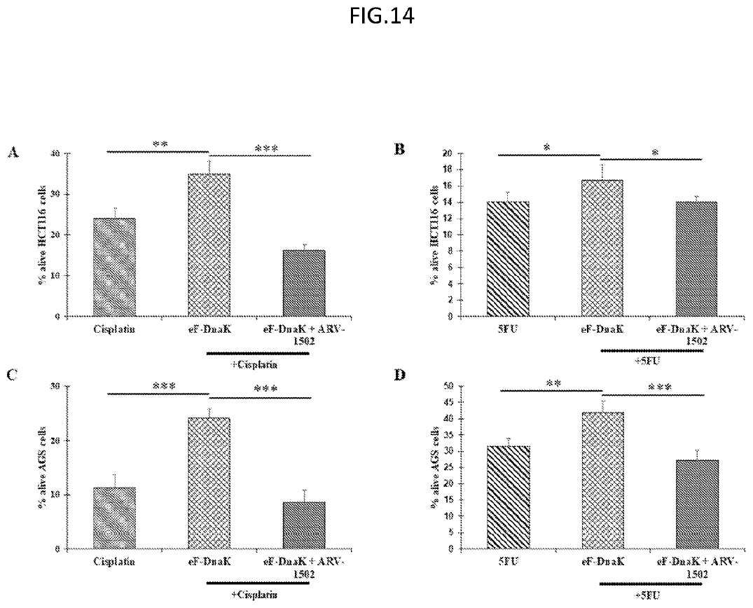

Given the similarity in amino acid composition between Mycoplasma and Fusobacterium DnaKs, we asked whether exogenously added F. nucleatum DnaK (eF-DnaK) could reduce the activity of cisplatin and 5FU. When cisplatin was used for the treatment of HCT116 cells in the presence of eF-DnaK, the anti-cancer effect of the drug was blunted and viability increased from 24% to 35% (FIG. 14A). Also the anti-cancer-effect of 5FU was reduced in the presence of eF-DnaK, and viability increased from 14% to 17% (FIG. 14B).

When AGS cells were treated with cisplatin in the presence of eF-DnaK, the anti-cancer effect was reduced and viability increased from 11% to 24% (FIG. 14C). Treatment with 5FU in the presence of eF-DnaK also reduced its anti-cancer effect, and viability increased from 31% to 42% (FIG. 14D).

Similarly to what observed with eM-DnaK, treatments with ARV-1502 were able to restore the drugs' anti-cancer activity in the presence of eF-DnaK (FIG. 14A-D, and also cf. FIG. 11A-D).

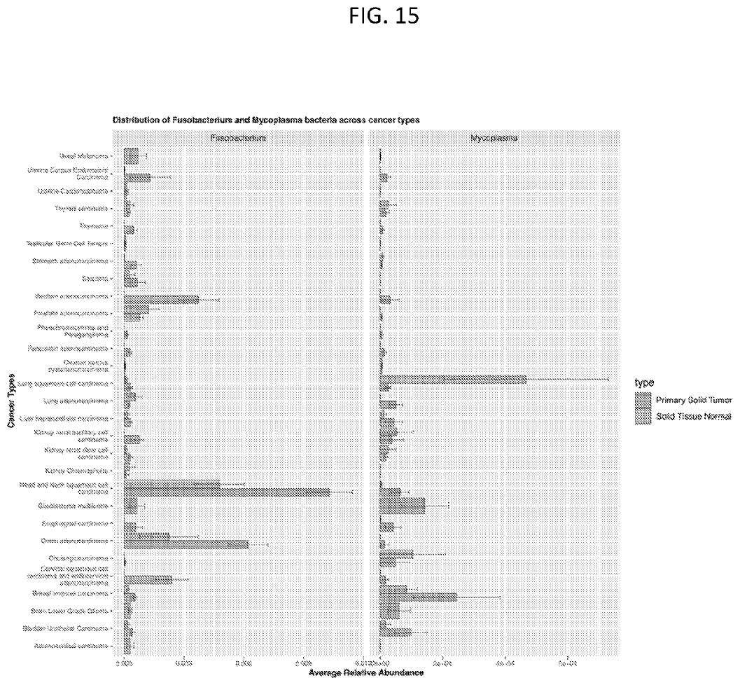

Next, we performed an analysis of bacteria associated with individual cancer types from the TCGA dataset. In some cases, we highlighted clear differences comparing solid tumor tissues to normal samples (FIG. 15). Fusobacterium is more frequently present in the primary solid tumor across all samples compared to normal tissues, except for a few types of cancers (namely, prostate adenocarcinoma, lung adenocarcinoma, and kidney chromophobe) where it was more abundant in the solid tissues normal (FIG. 15, left panel). To note, Fusobacterium is particularly present in the cancers related to the gastrointestinal tract (head and neck squamous cell carcinoma, esophageal carcinoma, colon adenocarcinoma and rectum adenocarcinoma). Mycoplasma was not as frequently observed as Fusobacterium, but still present more in the solid tumor tissues compared to the normal tissues adjacent to the tumor site (FIG. 15, right panel). As observed before, Mycoplasma also showed high abundance in the cancer tissues belonging to the gastrointestinal tract. On the other hand, lung squamous cell carcinoma and cholangiocarcinoma presented higher abundance of Mycoplasmas in the normal tissues compared to the primary solid tumor (FIG. 15, right panel).

Finally, although overall concordance between the TGCA data set and in vivo findings has been previously reported, we decided to further validate our analysis by assessing the presence of both Mycoplasma and Fusobacterium DnaKs in primary cancer tissues samples of both stomach and colon adenocarcinoma (Poore et al., Nature, (2020), 579:567-74). Both bacteria were readily detected by quantitative real-time PCR with specific primers and probe, in both tumor tissues in variable amount (Table 2).

TABLE 2
qPCR shows variable copy number of Mycoplasma and Fusobacterium
DnaK in primary cells from colon and stomach cancers.

| | | M-DnaK DNA | F-DnaK DNA |
|---|---|---|---|
| | | copies/50 ng | copies/50 ng |
| Patient ID | Tumor Site | total DNA | total DNA |
| 30284 | Colon | 95100 | 4800 |
| 30357 | Colon | 10000 | 1000 |
| P0859 | Colon | 3720 | 2580 |
| 30549 | Colon | ND | 3280 |
| P0860 | Colon | 3640 | ND |
| 30304 | Stomach | 15940 | 1140 |
| P0861 | Stomach | 296560 | 2880 |
| P0862 | Stomach | 3400 | 860 |
| 30404 | Stomach | 81560 | 15760 |

(ND: not detected)

Table 2 shows variable copy number of Mycoplasma and Fusobacterium DnaK in primary cells from colon and stomach cancers. (ND: not detected)

### DISCUSSION

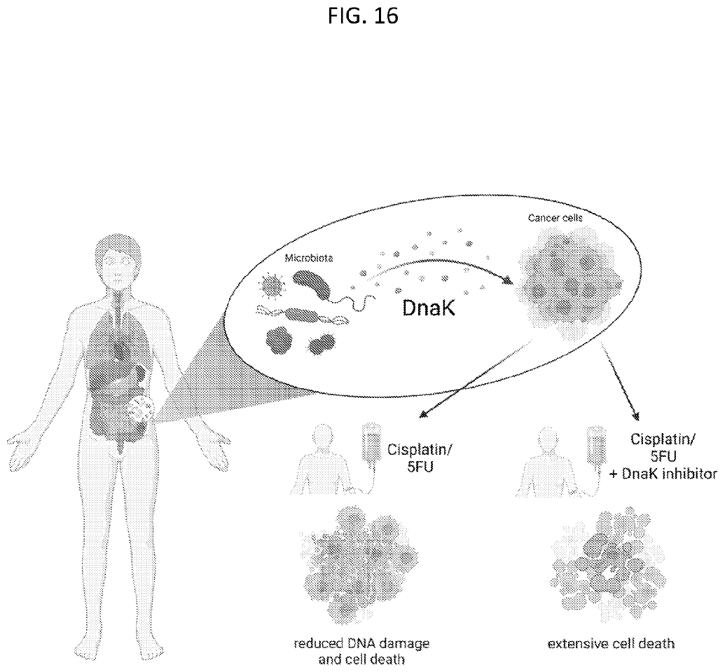

Here we show that Mycoplasma DnaK inhibits the anti-cancer effects of widely used anti-cancer drugs (cisplatin and 5FU) in HCT116 and AGS, colorectal and gastric carcinoma cell lines, respectively. We also show that a DnaK binding peptide (ARV-1502) can fully reverse this inhibitory effect. These data were confirmed in a spontaneous murine primary tumor from a knock-in mouse model constitutively expressing DnaK generated in our laboratory. Subsequently, by analyzing and comparing the distribution of bacteria in human cancer sequencing data sets obtained from TCGA, we identified several other CAB with DnaK highly similar to Mycoplasma DnaK in amino acid composition, suggesting their involvement in reducing the efficacy of chemotherapy. Among them, we identified F. nucleatun, and we provide evidence that also its DnaK reduces both cisplatin and 5FU anti-cancer activity, in turn restored by ARV-1502 (FIG. 16).

The use in this study of data from TCGA provides an additional layer of clinical relevance to our findings and further help to elucidate the role of specific components of the human cancer microbiota, namely Mycoplasma and Fusobacterium. We note that these bacteria were previously shown to reduce the anti-cancer activity of drugs like cisplatin and/or 5FU both in vitro and in vivo (Liu et al., PLoS One, (2017), 12:e0184578; Zhang et al., Journal of Experimental & Clinical Cancer Research, (2019), 38:14; Yu et al., Cell, (2017), 170:548-63 e16; Gethings-Behncke et al., Cancer Epidemiology, Biomarkers & Prevention, (2020), 29:539-48). Indeed, in cancer patients the levels of F. nucleatum predicted therapeutic response to chemotherapy, though the molecular mechanisms responsible for this effect are still largely unknown (Yamamura et al., Clinical Cancer Research, (2019), 25:6170-9).

Based on our data, we propose that DnaK reaches the intracellular compartments by two routes: i) taken up by cancer cells after being expressed and secreted by bacteria present in the tumor microenvironment, and ii) by being expressed and secreted inside tumor cells by invading bacteria like Mycoplasmas or Fusobacteria (Benedetti et al., International journal of molecular sciences, (2020), 21(4); Curreli et al., International Journal of Molecular Sciences, (2021), 22:3885; Zella et al., Proceedings of the National Academy of Sciences, (2018), 115:E12005; Taylor-Robinson et al., International journal of experimental pathology, (1991), 72:705-14; Brennan et al., Nature reviews Microbiology, (2019), 17:156-66). Intracellular DnaK then binds and reduces the activity of host proteins (such as p53) involved in the response to certain anti-cancer drugs (Zella et al., Proceedings of the National Academy of Sciences, (2018), 115:E12005-E14; Benedetti et al., International journal of molecular sciences, (2020), 21(4); Curreli et al., International Journal of Molecular Sciences, (2021), 22:3885). DnaK interaction with co-chaperone proteins, including the co-chaperone DnaJ, could provide the necessary ATPase activity for efficiently “sample” client substrates and function as a chaperone inside the eukaryotic cell (Zella et al., Proceedings of the National Academy of Sciences, (2018), 115:E12005-E14; Clerico et al., J Mol Biol. 2015; 427(7):1575-88). Since ARV-1502 binds the DnaK ATP-ase region and inhibits the ATP-ase activity, the peptide likely then acts by “locking” DnaK in a conformation unable to bind and inhibit the client proteins' functions, thus restoring the drugs' anti-cancer activity (Kragol et al., Biochemistry, (2001), 40:3016-26; Ostorhazi et al., Biopolymers, (2011), 96:126-9).

In conclusion, our study reveals a significant finding that two CAB, M. fermentans and F. nucleatum, use a novel mechanism to reduce the efficacy of anti-cancer drugs, and the use of TCGA data provides a broader context for the clinical significance of these findings. This discovery offers a practical framework for designing and implementing novel personalized anti-cancer strategies by targeting specific bacterial DnaKs in patients with poor response to chemotherapy.

### Example 6. DnaK Inhibitors Restore the Activity of Anticancer Drugs In Vivo

The role of DnaK, a protein produced by certain bacteria, is investigated in reducing the efficacy of anticancer drugs such as cisplatin and 5FU and it is determined whether DnaK inhibitors can restore their activity in vivo. In this experiment, cancer cells are injected into immunocompromised mice and the effects of the anticancer drugs in the presence or absence of DnaK inhibitors are evaluated. By using DnaK knock-in negative mice with a functional immune system, the experiment can investigate whether tumors can be successfully engrafted in syngeneic hosts. The objective is to unveil a novel bacterial-related DnaK-dependent mechanism of anticancer drug resistance and provide a target for diagnostic and therapeutic intervention in cancer patients with a poor response to chemotherapy. This research follows in vitro experiments on the role of DnaK in cancer, which showed that exogenous DnaK counteracted the efficacy of anticancer drugs and that DnaK inhibitors were able to restore their activity.

Cancer cells are injected into adult (4-24 weeks) C57BL/6 DnaK knock-in negative mice, nude mice, and SCID mice. The cancer cells are obtained from a spontaneous tumor originated from DnaK knock-in positive mice and are prepared as a single-cell suspension using a standard protocol. The tumor cells are immortalized (spontaneous or using immortalizing agents like SV40 or hTERT). Finally, tumor cells are injected either subcutaneously, intraperitoneally, or intravenously, and tumor growth is monitored regularly.

When the tumor size reaches about 5-6 mm in diameter (expected within 10-14 days), the mice are divided into four groups (n=10 for each group). One group is left untreated (control group A). The second group is treated with cisplatin+5FU via intraperitoneal injection to evaluate the efficacy of the drug on the tumor expressing DnaK. The third group is both treated with cisplatin/5FU and DnaK inhibitors to evaluate the efficacy of the drugs on the tumor expressing DnaK during DnaK inhibition. The fourth group is treated with DnaK inhibitors.

The animals in the third group receive DnaK inhibitors daily for two days after tumor engraftment is assessed, followed by cisplatin/5FU treatment. The treatment with DnaK inhibitors continues for four further days after the cisplatin/5FU injection. The animals in the second and third groups receive a single intraperitoneal (or intravenous) injection of cisplatin/5FU. The tumor size is checked twice a week, and when it reaches 20 mm in diameter (expected mostly in control groups and partially in the animals treated only with the anticancer drugs), the mice are euthanized, and the tumor size can be recorded. Additional experiments can be performed via intra-vein injection (tail vein).

These experiments can demonstrate the role of DnaK in reducing the efficacy of anticancer drugs in vivo; identify DnaK as a novel bacterial-related mechanism of anticancer drug resistance; validate DnaK inhibitors as potential therapeutic agents to restore the activity of anticancer drugs in cancer patients with a poor response to chemotherapy; determine the effects of cisplatin/5FU treatment alone and in combination with DnaK inhibitors on tumor growth in vivo; characterize tumor engraftment in syngeneic hosts using DnaK knock-in negative mice; and establish a preclinical model for evaluating the efficacy of DnaK inhibitors in combination with anticancer drugs in vivo.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A method for increasing the efficacy of an anticancer therapy in a subject, comprising administering to the subject in need thereof an effective amount of at least one compound that at least partially blocks the activity of a bacterial DnaK.

2. The method of claim 1, wherein the compound blocks the interaction of DnaK and PARP1.

3. The method of claim 1, wherein the compound blocks the interaction of DnaK and USP10.

4. The method of claim 1, wherein the compound inhibits the expression of DnaK.

5. The method of claim 1, wherein the compound is selected from telaprevir (also known as VX-950) and zafirlukast.

6. The method of claim 1, wherein the compound is a peptide comprising an amino acid sequence selected from GNNRPVYIPQPRPPHPRL and (SEQ ID NO:1) VDKGSYLPRPTPPRPIYNRN (SEQ ID NO:2) and variants thereof.

7. The method of claim 1, wherein the compound is selected from the group consisting of a DnaK antibody, and a nucleic acid that inhibits the expression of DnaK, wherein the nucleic acid is a RNA, a DNA, or a combination thereof, wherein the antibody is a monoclonal antibody.

8. (canceled)

9. (canceled)

10. (canceled)

11. (canceled)

12. (canceled)

13. The method of claim 7, wherein the nucleic acid is delivered by a bacterial virus.

14. (canceled)

15. The method of claim 1, wherein the compound is administered to a subject by topical, intravenous, subcutaneous, intramuscular, intracutaneous, transcutaneous, intrathecal, intranasal, intra-arterial, rectal, intragastric, parenteral, or oral administration.

16. The method of claim 1, further comprising administering the anticancer agent to the subject.

17. The method of claim 1, wherein the anticancer agent is selected from the group consisting of oxaliplatin, cisplatin, docetaxel, etoposide, palbociclib, lenalidomide, and bortezamib.

18. The method of claim 1, wherein the bacterial DnaK is one or more of DnaK from Mycoplasma, Mycoplasma fermentans, Heliobacter pylori, Fusobacterium nucleatum, and Chlamydia trachomatis.

19. The method of claim 1, wherein the method further comprises detection of a bacteria prior to administering to the subject in need thereof an effective amount of at least one compound that at least partially blocks the activity of a bacterial DnaK.

20. The method of claim 19, wherein the bacteria is selected from Mycoplasma, Mycoplasma fermentans, Heliobacter pylori, Fusobacterium nucleatum, Chlamydia trachomatis, and combinations thereof.

21. The method of claim 1, wherein the method further comprises detection of a bacterial DnaK in the subject prior to administering to the subject in need thereof an effective amount of at least one compound that at least partially blocks the activity of a bacterial DnaK.

22. The method of claim 21, wherein the bacterial DnaK is one or more of DnaK from Mycoplasma, Mycoplasma fermentans, Heliobacter pylori, Fusobacterium nucleatum, and Chlamydia trachomatis.

23. A pharmaceutical composition formulated for administration as an infusion, comprising an effective amount of at least one compound that at least partially blocks the activity of a bacterial DnaK, in combination with a pharmaceutically acceptable carrier.

24. The pharmaceutical composition of claim 23, wherein the at least one compound that at least partially blocks the activity of a bacterial DnaK is selected from the group consisting of telaprevir (also known as VX-950), zafirlukast, and combinations thereof.

25. The pharmaceutical composition of claim 23, further comprising an effective amount of an anticancer agent.

26. The pharmaceutical composition of claim 25, wherein the anticancer agent is selected from the group consisting of oxaliplatin, cisplatin, docetaxel, etoposide, palbociclib, lenalidomide, bortezamib, and combinations thereof.


SEQUENCE LISTING (23 sequences)

SEQ ID NO: 1 - protein, 18 aa, synthetic construct
GNNRPVYIPQPRPPHPRL

SEQ ID NO: 2 - protein, 20 aa, synthetic construct
VDKGSYLPRPTPPRPIYNRN

SEQ ID NO: 3 - DNA, 21 bp, synthetic construct
  source 1..21: mol_type = other DNA
tccctttcttgacatgctttg

SEQ ID NO: 4 - DNA, 22 bp, synthetic construct
  source 1..22: mol_type = other DNA
cgcctaatttaagaatggttgg

SEQ ID NO: 5 - DNA, 23 bp, synthetic construct
  source 1..23: mol_type = other DNA
acaatgcacaacgtgaagccaca

SEQ ID NO: 6 - DNA, 25 bp, synthetic construct
  source 1..25: mol_type = other DNA
tgctaaagcagcagcagtaggttcg

SEQ ID NO: 7 - DNA, 20 bp, synthetic construct
  source 1..20: mol_type = other DNA
gacaatgcacaacgtgaagc

SEQ ID NO: 8 - DNA, 20 bp, synthetic construct
  source 1..20: mol_type = other DNA
tcagcagcagcttttagacg

SEQ ID NO: 9 - DNA, 20 bp, synthetic construct
  source 1..20: mol_type = other DNA
gacaatgcacaacgtgaagc

SEQ ID NO: 10 - DNA, 20 bp, synthetic construct
  source 1..20: mol_type = other DNA
acgtgttgaaccaccaacaa

SEQ ID NO: 11 - DNA, 20 bp, synthetic construct
  source 1..20: mol_type = other DNA
gcaatggctcgtctaaaagc

SEQ ID NO: 12 - DNA, 20 bp, synthetic construct
  source 1..20: mol_type = other DNA
ctgcaagaacagcaccttga

SEQ ID NO: 13 - DNA, 22 bp, synthetic construct
  source 1..22: mol_type = other DNA
tgggtgctgcaattcaaggtgc

SEQ ID NO: 14 - DNA, 23 bp, synthetic construct
  source 1..23: mol_type = other DNA
gcacgttttgcatcagcttcacg

SEQ ID NO: 15 - DNA, 21 bp, synthetic construct
  source 1..21: mol_type = other DNA
tcgcaactctagatgcaggat

SEQ ID NO: 16 - DNA, 21 bp, synthetic construct
  source 1..21: mol_type = other DNA
aaacgagttgcttgttctgct

SEQ ID NO: 17 - DNA, 22 bp, synthetic construct
  source 1..22: mol_type = other DNA
acggcttttccgtttttgtctt

SEQ ID NO: 18 - DNA, 21 bp, synthetic construct
  source 1..21: mol_type = other DNA
tgcatccatgaaccgtatcca

SEQ ID NO: 19 - DNA, 21 bp, synthetic construct
  source 1..21: mol_type = other DNA
agcaatggcttttggtgatgc

SEQ ID NO: 20 - DNA, 20 bp, synthetic construct
  source 1..20: mol_type = other DNA
tgcattggacaggcaaacga

SEQ ID NO: 21 - DNA, 20 bp, synthetic construct
  source 1..20: mol_type = other DNA
agatgggacattagacggga

SEQ ID NO: 22 - DNA, 20 bp, synthetic construct
  source 1..20: mol_type = other DNA
tcgcgaggacttaccaacat

SEQ ID NO: 23 - protein, 585 aa, Mycoplasma fermentans
MAKETIIGIDLGTTNSAVAIVDGGTPIVLENYNGKRTTPSVVSFKDGEIIVGENAKNQIETNPDTIASVKRFMGTKKIFKANGKEYKPEEISAIILDHLRKYAEEKVGHKIEKAVITVPAYFDNAQREATKIAGKIAGLDVLRIINEPTAAALAFGLDKTNKEMKVLVFDLGGGTFDVSILELADGTFEVLATSGDNKLGGDDWDHEIVDWLVAKIKNDHKIDIRENKMAMARLKAAAEKAKIDLSSSLVAHISLPFLVLLDNHEPINVEAELKRSEFEKMTAKLVERCRRPIQDALSEAKLKISDLDEILLVGGSTRIPAVQALVEKILNRKPNKSVNPDEVVAMGAAIQGAVLAGDINDILLVDVTPLTLGIETAGGISTPLIPRNTRIPITKSETFTTFENNQTDVTIKIVQGERPVASENKLLGQFNLTGIRPAPRGIPQIEVSFKIDANGITTVSAKDKDTQKEQSITIKNSSKLSEEEVERMIKEAEENREADAKRAADIEIIVRAETMVAKFESVLEENKDKLTQDQINQAQAEIDKINGFIKEKEYDQLRLTIKAFEELLDSMSNADSSSFKEEDAE